SEQUENCE LISTING (4 sequences)

SEQ ID NO: 1 - protein, 231 aa, Artificial Sequence
  Description of Artificial Sequence Synthetic polypeptide
EVQLVESGGGLVQPGGSLRLSCAASGYTFTNYGMNWVRQAPGKGLEWVGWINTYTGEPTYAADFKRRFTFSLDTSKSTAYLQMNSLRAEDTAVYYCAKYPHYYGSSHWYFDVWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHT

SEQ ID NO: 2 - protein, 214 aa, Artificial Sequence
  Description of Artificial Sequence Synthetic polypeptide
DIQMTQSPSSLSASVGDRVTITCSASQDISNYLNWYQQKPGKAPKVLIYFTSSLHSGVPSRFSGSGSGTDFTLTISSLQPEDFATYYCQQYSTVPWTFGQGTKVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 3 - protein, 231 aa, Artificial Sequence; identical to SEQ ID NO: 1
  Description of Artificial Sequence Synthetic polypeptide

SEQ ID NO: 4 - protein, 214 aa, Artificial Sequence; identical to SEQ ID NO: 2
  Description of Artificial Sequence Synthetic polypeptide

(19) United States
(12) Patent Application Publication
(10) Pub. No.: US 2014/0178366 A1
(43) Pub. Date: Jun. 26, 2014

(54) PRESELECTION OF SUBJECTS FOR THERAPEUTIC TREATMENT BASED ON HYPOXIC STATUS

(71) Applicants: Ronald K. Blackman, Brookline, MA (US); Vojo Vukovic, Winchester, MA (US)

(72) Inventors: Ronald K. Blackman, Brookline, MA (US); Vojo Vukovic, Winchester, MA (US)

(73) Assignee: SYNTA PHARMACEUTICALS CORP., Lexington, MA (US)

(21) Appl. No.: 14/109,543

(22) Filed: Dec. 17, 2013

Related U.S. Application Data
(63) Continuation of application No. 13/300,221, filed on Nov. 18, 2011, now abandoned.
(60) Provisional application No. 61/510,660, filed on Jul. 22, 2011.
(60) Provisional application No. 61/510,653, filed on Jul. 22, 2011.
(60) Provisional application No. 61/510,648, filed on Jul. 22, 2011.
(60) Provisional application No. 61/415,122, filed on Nov. 18, 2010.
(60) Provisional application No. 61/415,136, filed on Nov. 18, 2010.
(60) Provisional application No. 61/415,139, filed on Nov. 18, 2010.
(60) Provisional application No. 61/415,147, filed on Nov. 18, 2010.
(60) Provisional application No. 61/415,155, filed on Nov. 18, 2010.
(60) Provisional application No. 61/415,156, filed on Nov. 18, 2010.
(60) Provisional application No. 61/415,158, filed on Nov. 18, 2010.

(51) Int. Cl. C07K 16/22; A61K 31/4985; A61K 31/517; G01N 33/574; A61K 31/4745; A61K 31/4439; A61K 31/506; A61K 31/502; A61K 31/439; A61K 31/44

(52) U.S. Cl. CPC C07K 16/22; A61K 31/439; A61K 31/4985; A61K 31/517; A61K 31/44; A61K 31/4745; A61K 31/4439; A61K 31/506; A61K 31/502; G01N 33/57484
USPC 424/133.1; 514/294; 514/249; 514/266.4; 514/346; 514/293; 514/338; 514/266.2; 514/275; 514/248; 435/29; 435/7.23; 435/6.12; 435/6.11; 435/7.92; 435/7.4; 435/15; 435/26

(57) ABSTRACT

The present invention provides methods for the preselection of a subject for therapeutic treatment with an agent based on modulated levels of hypoxia in cancerous cells in the subject. In one embodiment, the invention provides methods for the preselection of a subject for therapeutic treatment with an agent based on modulated levels of lactate dehydrogenase (LDH) in a cell, e.g., a cancerous cell. The invention also provides methods for treating cancer in a subject by administering an effective amount of an agent to the subject, wherein the subject has been selected based on a modulated level of hypoxia. The invention further provides kits to practice the methods of the invention.

### CROSS REFERENCE TO RELATED APPLICATIONS

This application claims priority to U.S. Provisional Patent Application Ser. Nos. 61/415,122, 61/415,136, 61/415,139, 61/415,147, 61/415,155, 61/415,156, and 61/415,158, all filed on Nov. 18, 2010; and U.S. Provisional Patent Application Ser. Nos. 61/510,660, 61/510,653, and 61/510,648, all filed on Jul. 22, 2011. Each application is incorporated herein by reference in its entirety.

### BACKGROUND OF THE INVENTION

As tumors grow, they begin to exceed their supply of oxygen. Hypoxia occurs when the growth of the tumor exceeds new blood vessel formation, and the tumor must undergo genetic and adaptive changes to allow it to survive and proliferate in a less well-oxygenated environment. In such a hypoxic microenvironment, tumors exhibit a greater dependency on certain signaling pathways, referred to as oxygen-sensitive pathways, to facilitate crucial adaptive mechanisms, such as angiogenesis, glycolysis, growth-factor signaling, immortalization, genetic instability, tissue invasion and metastasis, apoptosis, and pH regulation (see, e.g., Harris, Nature Reviews, 2:38-47, 2002).

A number of oxygen-sensitive pathways have been shown to be regulated by hypoxia, including hypoxia-inducible factor (HIF) pathways, vascular endothelial growth factor (VEGF) pathways, and mammalian target of rapamycin (mTOR) pathways. See e.g., Melillo, Cancer Metastasis Rev 26: 341-352, 2007. Hypoxia has also been shown to upregulate epidermal growth factor receptor (EGFR) expression in tumors (Franovic et al., PNAS 104:13092-13097, 2007), which then leads to phosphorylation of tyrosine residues in the kinase domain of the receptor and activation of the Ras/Maf/MAPK or PI3K/Akt/mTOR pathways. Activation of these oxygen-sensitive pathways results in the nuclear activation of genes related to angiogenesis, cell proliferation, growth, metastasis, and adhesion (Langer and Soria, Clin. Lung Cancer, 11(2) 82-90, 2010).

Therapeutic agents targeting these oxygen-sensitive pathways are invaluable for the treatment of diseases such as cancer. However, patient response to currently available therapeutic agents is not always predictable. Indeed, although research has provided physicians with ever more options for therapeutics for the treatment of cancer, the ability to match a therapeutic agent to a specific patient based not just on the site of the tumor, but the characteristic of the tumor, is lacking. Accordingly, a need exists for the accurate prediction of patient response to currently available therapeutic agents.

### SUMMARY OF THE INVENTION

The instant invention surprisingly demonstrates that high levels of hypoxia in a subject can be used to predict whether a patient will respond to treatment with an agent selected from the group consisting of bevacizumab, ganetespib, temsirolimus, erlotinib, PTK787, BEZ235, XL765, pazopanib, cediranib, and axitinib. Specifically, the present invention provides methods for the preselection of a subject for therapeutic treatment with an agent based on high levels of hypoxia in cancerous cells in the subject. In one embodiment, the invention provides methods for the preselection of a subject for therapeutic treatment with a selected agent based on high levels of lactate dehydrogenase (LDH) in a cell, e.g., a cancerous cell. The invention also provides methods for treating cancer in a subject by administering an effective amount of a selected agent to the subject, wherein the subject has been selected based on a high level of hypoxia. The invention further provides kits to practice the methods of the invention.

The invention provides compositions for use in methodsof treating a subjects having cancer, the composition comprising an agent inlcuding bevacizumab, ganetespib, temsirolimus, erlotinib, PTK787, BEZ235, XL765, pazopanib, cediranib, and axitinib, wherein the cancer comprises a tumor with a high level of hypoxia.

In certain embodiments, the cancer is a solid tumor. In certain embodiments, the cancer is a blood tumor, i.e., not a solid tumor. The type of cancer includes, but is not limited to, one or more of the cancer types such as primary cancer, metastatic cancer, breast cancer, colon cancer, rectal cancer, lung cancer, oropharyngeal cancer, hypopharyngeal cancer, esophageal cancer, stomach cancer, pancreatic cancer, liver cancer, gallbladder cancer, bile duct cancer, small intestine cancer, urinary tract cancer, kidney cancer, bladder cancer, urothelium cancer, female genital tract cancer, cervical cancer, uterine cancer, ovarian cancer, choriocarcinoma, gestational trophoblastic disease, male genital tract cancer, prostate cancer, seminal vesicle cancer, testicular cancer, germ cell tumors, endocrine gland tumors, thyroid cancer, adrenal cancer, pituitary gland cancer, skin cancer, hemangiomas, melanomas, sarcomas arising from bone and soft tissues, Kaposi's sarcoma, brain cancer, nerve cancer, ocular cancer, meningial cancer, astrocytoma, glioma, glioblastoma, retinoblastoma, neuroma, neuroblastoma, Schwannoma, meningioma, solid tumors arising from hematopoietic malignancies, leukemia, Hodgkin's lymphoma, non-Hodgkin's lymphoma, Burkitt's lymphoma, metastatic melanoma, recurrent or persistent ovarian epithelial cancer, fallopian tube cancer, primary peritoneal cancer, epithelial ovarian cancer, primary peritoneal serous cancer, non-small cell lung cancer, gastrointestinal stromal tumors, colorectal cancer, small cell lung cancer, melanoma, glioblastoma multiforme, non-squamous non-small-cell lung cancer, malignant glioma, primary peritoneal serous cancer, metastatic liver cancer, neuroendocrine carcinoma, refractory malignancy, triple negative breast cancer, HER2 amplified breast cancer, squamous cell carcinoma, nasopharageal cancer, oral cancer, biliary tract, hepatocellular carcinoma, squamous cell carcinomas of the head and neck (SCCHN), non-medullary thyroid carcinoma, neurofibromatosis type 1, CNS cancer, liposarcoma, leiomyosarcoma, salivary gland cancer, mucosal melanoma, acral/lentiginous melanoma, paraganglioma; pheochromocytoma, advanced metastatic cancer, solid tumor, squamous cell carcinoma, sarcoma, melanoma, endometrial cancer, head and neck cancer, rhabdomysarcoma, multiple myeloma, gastrointestinal stromal tumor, mantle cell lymphoma, gliosarcoma, bone sarcoma, and refractory malignancy.

In certain embodiments, the level of hypoxia in a tumor is determined in a subject sample. The subject sample can include, but is not limited to, one or more of tumor tissue, blood, urine, stool, lymph, cerebrospinal fluid, circulating tumor cells, bronchial lavage, peritoneal lavage, exudate, effusion, and sputum. In certain embodiments, the tumor tissue is tumor tissue that is in the subject or that is removed from the subject.

In certain embodiments, the level of hypoxia is determined by detecting the activity level or expression level of one or more hypoxia modulated polypeptides. In certain embodiments, the activity level or expression level of the one or more hypoxia modulated polypeptides are up regulated in the sample. The level of hypoxia can be determined by any method known in the art including, but not limited to, detecting the activity level or expression level of one or more hypoxia modulated polypeptides or using detection methods selected from the group consisting of detection of activity or expression of at least one isoform or subunit of lactate dehydrogenase (LDH), at least one isoform or subunit of hypoxia inducible factor (HIF), at least one pro-angiogenic form of vascular endothelial growth factor (VEGF), phosphorylated VEGF receptor (pKDR) 1, 2, and 3; neurolipin 1 (NRP-1), pyruvate dehydrokinase (PDH-K), ornithine decarboxylase (ODC), glucose transporter-1 (GLUT-1), glucose transporter-2 (GLUT-2), tumor size, blood flow, EF5 binding, pimonidazole binding, PET scan, and probe detection of hypoxia level.

In certain embodiments, the isoform or subunit of LDH comprises one or more selected from the group consisting of, LDH5, LDH4, LDH3, LDH2, LDH1, LDHA and LDHB; or any combination thereof including total LDH. In certain embodiments, the isoform of HIF comprises one or more selected from the group consisting of HIF-1α, HIF-1β, HIF-2α, and HIF-2β; or any combination thereof including total HIF-1 and/or HIF-2. In certain embodiments, the pro-angiogenic isoform of VEGF is any VEGF-A isoform, or any combination of VEGF-A isoforms including total VEGF-A.

In certain embodiments, detection of a high level of activity or expression of at least one LDH isoform or subunit comprises detection of an LDH activity or expression level of an LDH selected from the group consisting of total LDH, LDH5, LDH4, LDH5 plus LDH4, LDH5 plus LDH4 plus LDH3, and LDHA, wherein the activity level or expression level is 0.8 ULN or more. In certain embodiments, detection of a high level of activity or expression of at least one LDH isoform or subunit comprises detection of an LDH activity or expression level of an LDH selected from the group consisting of total LDH, LDH5, LDH4, LDH5 plus LDH4, LDH5 plus LDH4 plus LDH3, and LDHA, wherein the activity level or expression level is 1.0 ULN or more.

In certain embodiments, detection of a high level of hypoxia comprises detection of a change in a ratio or levels of activity or expression or a change in a ratio of normalized levels of activity or expression of hypoxia modulated polypeptides. In certain embodiments, a high level of hypoxia comprises a ratio or a normalized ratio of 1.0 or more of the ULN, wherein the ratio or normalized ratio is selected from the group consisting of the LDHA to LDHB, LDH5 or LDH4 to LDH1, LDH5 or LDH4 to total LDH, LDH5 and LDH4 to LDH1, LDH5 and LDH4 to total LDH, LDH5, LDH4, and LDH3 to LDH1, and LDH5, LDH4, and LDH3 to total LDH.

In certain embodiments, the subject was previously treated with another chemotherapeutic agent. In certain embodiments, the method further includes identifying a subject as having a high level of hypoxia.

The invention provides methods and use of a level of hypoxia in a tumor for identifying a subject for treatment with an agent including bevacizumab, ganetespib, temsirolimus, erlotinib, PTK787, BEZ235, XL765, pazopanib, cediranib, and axitinib by determining the level of hypoxia in a tumor from the subject, wherein a high level of hypoxia in the sample indicates the subject is likely to respond to therapy with an agent such as bevacizumab, ganetespib, temsirolimus, erlotinib, PTK787, BEZ235, XL765, pazopanib, cediranib, and axitinib.

In certain embodiments, a subject having a low level of hypoxia in the tumor is not likely to respond to therapy with an agent selected from the group consisting of bevacizumab, ganetespib, temsirolimus, erlotinib, PTK787, BEZ235, XL765, pazopanib, cediranib, and axitinib.

In certain embodiments, the cancer is a solid tumor. In certain embodiments, the cancer is a blood tumor, i.e., not a solid tumor. The type of cancer includes, but is not limited to, one or more of the cancer types provided herein.

In certain embodiments, the level of hypoxia in a tumor is determined in a subject sample. The subject sample can include, but is not limited to, one or more of tumor tissue, blood, urine, stool, lymph, cerebrospinal fluid, circulating tumor cells, bronchial lavage, peritoneal lavage, exudate, effusion, and sputum. In certain embodiments, the tumor tissue is tumor tissue that is in the subject or that is removed from the subject.

In certain embodiments, the level of hypoxia is determined by detecting the activity level or expression level of one or more hypoxia modulated polypeptides. In certain embodiments, the activity level or expression level of the one or more hypoxia modulated polypeptides are up regulated in the sample. The level of hypoxia can be determined by any method known in the art including, but not limited to, detecting the activity level or expression level of one or more hypoxia modulated polypeptides or using detection methods selected from the group consisting of detection of activity or expression of at least one isoform or subunit of lactate dehydrogenase (LDH), at least one isoform or subunit of hypoxia inducible factor (HIF), at least one pro-angiogenic form of vascular endothelial growth factor (VEGF), phosphorylated VEGF receptor (pKDR) 1, 2, and 3; neurolipin 1 (NRP-1), pyruvate dehydrokinase (PDH-K), ornithine decarboxylase (ODC), glucose transporter-1 (GLUT-1), glucose transporter-2 (GLUT-2), tumor size, blood flow, EF5 binding, pimonidazole binding, PET scan, and probe detection of hypoxia level.

In certain embodiments, the isoform or subunit of LDH comprises one or more selected from the group consisting of, LDH5, LDH4, LDH3, LDH2, LDH1, LDHA and LDHB; or any combination thereof including total LDH. In certain embodiments, the isoform of HIF comprises one or more selected from the group consisting of HIF-1α, HIF-1β, HIF-2α, and HIF-2β; or any combination thereof including total HIF-1 and/or HIF-2. In certain embodiments, the pro-angiogenic isoform of VEGF is any VEGF-A isoform, or any combination of VEGF-A isoforms including total VEGF-A.

In certain embodiments, detection of a high level of activity or expression of at least one LDH isoform or subunit comprises detection of an LDH activity or expression level of an LDH selected from the group consisting of total LDH, LDH5, LDH4, LDH5 plus LDH4, LDH5 plus LDH4 plus LDH3, and LDHA, wherein the activity level or expression level is 0.8 ULN or more. In certain embodiments, detection of a high level of activity or expression of at least one LDH isoform or subunit comprises detection of an LDH activity or expression level of an LDH selected from the group consisting of total LDH, LDH5, LDH4, LDH5 plus LDH4, LDH5 plus LDH4 plus LDH3, and LDHA, wherein the activity level or expression level is 1.0 ULN or more.

In certain embodiments, detection of a high level of hypoxia comprises detection of a change in a ratio or levels of activity or expression or a change in a ratio of normalized levels of activity or expression of hypoxia modulated polypeptides. In certain embodiments, a high level of hypoxia comprises a ratio or a normalized ratio of 1.0 or more of the ULN, wherein the ratio or normalized ratio is selected from the group consisting of the LDHA to LDHB, LDH5 or LDH4 to LDH1, LDH5 or LDH4 to total LDH, LDH5 and LDH4 to LDH1, LDH5 and LDH4 to total LDH, LDH5, LDH4, and LDH3 to LDH1, and LDH5, LDH4, and LDH3 to total LDH.

In certain embodiments, the subject was previously treated with another chemotherapeutic agent.

The invention provides tests, methods of testing, and the use of a level of hypoxia for the manufacture of a test to select a therapeutic regimen including an agent selected from the group consisting of bevacizumab, ganetespib, temsirolimus, erlotinib, PTK787, BEZ235, XL765, pazopanib, cediranib, and axitinib for the treatment of cancer comprising at least one reagent for determining the level of hypoxia of in a subject sample; wherein the level of hypoxia is used to select the treatment regimen including an agent selected from the group consisting of bevacizumab, ganetespib, temsirolimus, erlotinib, PTK787, BEZ235, XL765, pazopanib, cediranib, and axitinib. Reagents for use in such tests can include, but are not limited to at least one agent specifically for detection of a level of hypoxia or determining the level of hypoxia in a subject such as an antibody for detection of the expression level of one or more oxygen sensitive peptides including antibodies specific for a phosphorylation state or otherwise modified state of an oxygen sensitive peptide, a substrate for one or more oxygen sensitive peptides, a nucleic acid for detection of the expression level of one or more oxygen sensitive peptides, and a control sample containing a known amount or concentration of an oxygen sensitive peptide and/or nucleic acid.

In certain embodiments, a subject having a high level of hypoxia in the tumor is likely to respond to therapy with an agent selected from the group consisting of bevacizumab, ganetespib, temsirolimus, erlotinib, PTK787, BEZ235, XL765, pazopanib, cediranib, and axitinib. In certain embodiments, a subject having a low level of hypoxia in the tumor is not likely to respond to therapy with an agent selected from the group consisting of bevacizumab, ganetespib, temsirolimus, erlotinib, PTK787, BEZ235, XL765, pazopanib, cediranib, and axitinib.

In certain embodiments, the cancer is a solid tumor. In certain embodiments, the cancer is a blood tumor, i.e., not a solid tumor. The type of cancer includes, but is not limited to, one or more of the cancer types provided herein.

In certain embodiments, the level of hypoxia in a tumor is determined in a subject sample. The subject sample can include, but is not limited to, one or more of tumor tissue, blood, urine, stool, lymph, cerebrospinal fluid, circulating tumor cells, bronchial lavage, peritoneal lavage, exudate, effusion, and sputum. In certain embodiments, the tumor tissue is tumor tissue that is in the subject or that is removed from the subject.

In certain embodiments, the level of hypoxia is determined by detecting the activity level or expression level of one or more hypoxia modulated polypeptides. In certain embodiments, the activity level or expression level of the one or more hypoxia modulated polypeptides are up regulated in the sample. The level of hypoxia can be determined by any method known in the art including, but not limited to, detecting the activity level or expression level of one or more hypoxia modulated polypeptides or using detection methods selected from the group consisting of detection of activity or expression of at least one isoform or subunit of lactate dehydrogenase (LDH), at least one isoform or subunit of hypoxia inducible factor (HIF), at least one pro-angiogenic form of vascular endothelial growth factor (VEGF), phosphorylated VEGF receptor (pKDR) 1, 2, and 3; neurolipin 1 (NRP-1), pyruvate dehydrokinase (PDH-K), ornithine decarboxylase (ODC), glucose transporter-1 (GLUT-1), glucose transporter-2 (GLUT-2), tumor size, blood flow, EF5 binding, pimonidazole binding, PET scan, and probe detection of hypoxia level.

In certain embodiments, the isoform or subunit of LDH comprises one or more selected from the group consisting of, LDH5, LDH4, LDH3, LDH2, LDH1, LDHA and LDHB; or any combination thereof including total LDH. In certain embodiments, the isoform of HIF comprises one or more selected from the group consisting of HIF-1α, HIF-1β, HIF-2α, and HIF-2β; or any combination thereof including total HIF-1 and/or HIF-2. In certain embodiments, the pro-angiogenic isoform of VEGF is any VEGF-A isoform, or any combination of VEGF-A isoforms including total VEGF-A.

In certain embodiments, detection of a high level of activity or expression of at least one LDH isoform or subunit comprises detection of an LDH activity or expression level of an LDH selected from the group consisting of total LDH, LDH5, LDH4, LDH5 plus LDH4, LDH5 plus LDH4 plus LDH3, and LDHA, wherein the activity level or expression level is 0.8 ULN or more. In certain embodiments, detection of a high level of activity or expression of at least one LDH isoform or subunit comprises detection of an LDH activity or expression level of an LDH selected from the group consisting of total LDH, LDH5, LDH4, LDH5 plus LDH4, LDH5 plus LDH4 plus LDH3, and LDHA, wherein the activity level or expression level is 1.0 ULN or more.

In certain embodiments, detection of a high level of hypoxia comprises detection of a change in a ratio or levels of activity or expression or a change in a ratio of normalized levels of activity or expression of hypoxia modulated polypeptides. In certain embodiments, a high level of hypoxia comprises a ratio or a normalized ratio of 1.0 or more of the ULN, wherein the ratio or normalized ratio is selected from the group consisting of the LDHA to LDHB, LDH5 or LDH4 to LDH1, LDH5 or LDH4 to total LDH, LDH5 and LDH4 to LDH1, LDH5 and LDH4 to total LDH, LDH5, LDH4, and LDH3 to LDH1, and LDH5, LDH4, and LDH3 to total LDH.

In certain embodiments, the subject was previously treated with another chemotherapeutic agent.

The invention provides methods and uses of an agent such as bevacizumab, ganetespib, temsirolimus, erlotinib, PTK787, BEZ235, XL765, pazopanib, cediranib, and axitinib for preparation of a medicament for treating a subject having cancer, wherein the subject has a tumor with a high level of hypoxia.

In certain embodiments, a subject having a low level of hypoxia in the tumor is not likely to respond to therapy with an agent selected from the group consisting of bevacizumab, ganetespib, temsirolimus, erlotinib, PTK787, BEZ235, XL765, pazopanib, cediranib, and axitinib.

In certain embodiments, the cancer is a solid tumor. In certain embodiments, the cancer is a blood tumor, i.e., not a solid tumor. The type of cancer includes, but is not limited to, one or more of the cancer types provided herein.

In certain embodiments, the level of hypoxia in a tumor is determined in a subject sample. The subject sample can include, but is not limited to, one or more of tumor tissue, blood, urine, stool, lymph, cerebrospinal fluid, circulating tumor cells, bronchial lavage, peritoneal lavage, exudate, effusion, and sputum. In certain embodiments, the tumor tissue is tumor tissue that is in the subject or that is removed from the subject.

In certain embodiments, the level of hypoxia is determined by detecting the activity level or expression level of one or more hypoxia modulated polypeptides. In certain embodiments, the activity level or expression level of the one or more hypoxia modulated polypeptides are up regulated in the sample. The level of hypoxia can be determined by any method known in the art including, but not limited to, detecting the activity level or expression level of one or more hypoxia modulated polypeptides or using detection methods selected from the group consisting of detection of activity or expression of at least one isoform or subunit of lactate dehydrogenase (LDH), at least one isoform or subunit of hypoxia inducible factor (HIF), at least one pro-angiogenic form of vascular endothelial growth factor (VEGF), phosphorylated VEGF receptor (pKDR) 1, 2, and 3; neurolipin 1 (NRP-1), pyruvate dehydrokinase (PDH-K), ornithine decarboxylase (ODC), glucose transporter-1 (GLUT-1), glucose transporter-2 (GLUT-2), tumor size, blood flow, EF5 binding, pimonidazole binding, PET scan, and probe detection of hypoxia level.

In certain embodiments, the isoform or subunit of LDH comprises one or more selected from the group consisting of, LDH5, LDH4, LDH3, LDH2, LDH1, LDHA and LDHB; or any combination thereof including total LDH. In certain embodiments, the isoform of HIF comprises one or more selected from the group consisting of HIF-1α, HIF-1β, HIF-2α, and HIF-2β; or any combination thereof including total HIF-1 and/or HIF-2. In certain embodiments, the pro-angiogenic isoform of VEGF is any VEGF-A isoform, or any combination of VEGF-A isoforms including total VEGF-A.

In certain embodiments, detection of a high level of activity or expression of at least one LDH isoform or subunit comprises detection of an LDH activity or expression level of an LDH selected from the group consisting of total LDH, LDH5, LDH4, LDH5 plus LDH4, LDH5 plus LDH4 plus LDH3, and LDHA, wherein the activity level or expression level is 0.8 ULN or more. In certain embodiments, detection of a high level of activity or expression of at least one LDH isoform or subunit comprises detection of an LDH activity or expression level of an LDH selected from the group consisting of total LDH, LDH5, LDH4, LDH5 plus LDH4, LDH5 plus LDH4 plus LDH3, and LDHA, wherein the activity level or expression level is 1.0 ULN or more.

In certain embodiments, detection of a high level of hypoxia comprises detection of a change in a ratio or levels of activity or expression or a change in a ratio of normalized levels of activity or expression of hypoxia modulated polypeptides. In certain embodiments, a high level of hypoxia comprises a ratio or a normalized ratio of 1.0 or more of the ULN, wherein the ratio or normalized ratio is selected from the group consisting of the LDHA to LDHB, LDH5 or LDH4 to LDH1, LDH5 or LDH4 to total LDH, LDH5 and LDH4 to LDH1, LDH5 and LDH4 to total LDH, LDH5, LDH4, and LDH3 to LDH1, and LDH5, LDH4, and LDH3 to total LDH.

In certain embodiments, the subject was previously treated with another chemotherapeutic agent.

The invention provides business methods for decreasing healthcare costs by determining the level of hypoxia in a biological sample from a tumor obtained from a subject; storing the information on a computer processor; determining if the subject would likely benefit from treatment with an agent selected from the group consisting of bevacizumab, ganetespib, temsirolimus, erlotinib, PTK787, BEZ235, XL765, pazopanib, cediranib, and axitinib based on the level of hypoxia; and treating the subject only if the subject will likely benefit from treatment, thereby decreasing healthcare costs.

In certain embodiments, a subject having a low level of hypoxia in the tumor is not likely to respond to therapy with an agent selected from the group consisting of bevacizumab, ganetespib, temsirolimus, erlotinib, PTK787, BEZ235, XL765, pazopanib, cediranib, and axitinib.

In certain embodiments, the cancer is a solid tumor. In certain embodiments, the cancer is a blood tumor, i.e., not a solid tumor. The type of cancer includes, but is not limited to, one or more of the cancer types provided herein.

In certain embodiments, the level of hypoxia in a tumor is determined in a subject sample. The subject sample can include, but is not limited to, one or more of tumor tissue, blood, urine, stool, lymph, cerebrospinal fluid, circulating tumor cells, bronchial lavage, peritoneal lavage, exudate, effusion, and sputum. In certain embodiments, the tumor tissue is tumor tissue that is in the subject or that is removed from the subject.

In certain embodiments, the level of hypoxia is determined by detecting the activity level or expression level of one or more hypoxia modulated polypeptides. In certain embodiments, the activity level or expression level of the one or more hypoxia modulated polypeptides are up regulated in the sample. The level of hypoxia can be determined by any method known in the art including, but not limited to, detecting the activity level or expression level of one or more hypoxia modulated polypeptides or using detection methods selected from the group consisting of detection of activity or expression of at least one isoform or subunit of lactate dehydrogenase (LDH), at least one isoform or subunit of hypoxia inducible factor (HIF), at least one pro-angiogenic form of vascular endothelial growth factor (VEGF), phosphorylated VEGF receptor (pKDR) 1, 2, and 3; neurolipin 1 (NRP-1), pyruvate dehydrokinase (PDH-K), ornithine decarboxylase (ODC), glucose transporter-1 (GLUT-1), glucose transporter-2 (GLUT-2), tumor size, blood flow, EF5 binding, pimonidazole binding, PET scan, and probe detection of hypoxia level.

In certain embodiments, the isoform or subunit of LDH comprises one or more selected from the group consisting of, LDH5, LDH4, LDH3, LDH2, LDH1, LDHA and LDHB; or any combination thereof including total LDH. In certain embodiments, the isoform of HIF comprises one or more selected from the group consisting of HIF-1α, HIF-1β, HIF-2α, and HIF-2β; or any combination thereof including total HIF-1 and/or HIF-2. In certain embodiments, the pro-angiogenic isoform of VEGF is any VEGF-A isoform, or any combination of VEGF-A isoforms including total VEGF-A.

In certain embodiments, detection of a high level of activity or expression of at least one LDH isoform or subunit comprises detection of an LDH activity or expression level of an LDH selected from the group consisting of total LDH, LDH5, LDH4, LDH5 plus LDH4, LDH5 plus LDH4 plus LDH3, and LDHA, wherein the activity level or expression level is 0.8 ULN or more. In certain embodiments, detection of a high level of activity or expression of at least one LDH isoform or subunit comprises detection of an LDH activity or expression level of an LDH selected from the group consisting of total LDH, LDH5, LDH4, LDH5 plus LDH4, LDH5 plus LDH4 plus LDH3, and LDHA, wherein the activity level or expression level is 1.0 ULN or more.

In certain embodiments, detection of a high level of hypoxia comprises detection of a change in a ratio or levels of activity or expression or a change in a ratio of normalized levels of activity or expression of hypoxia modulated polypeptides. In certain embodiments, a high level of hypoxia comprises a ratio or a normalized ratio of 1.0 or more of the ULN, wherein the ratio or normalized ratio is selected from the group consisting of the LDHA to LDHB, LDH5 or LDH4 to LDH1, LDH5 or LDH4 to total LDH, LDH5 and LDH4 to LDH1, LDH5 and LDH4 to total LDH, LDH5, LDH4, and LDH3 to LDH1, and LDH5, LDH4, and LDH3 to total LDH.

In certain embodiments, the subject was previously treated with another chemotherapeutic agent.

The invention provides methods for identifying a subject for treatment with an agent selected from the group consisting of bevacizumab, ganetespib, temsirolimus, erlotinib, PTK787, BEZ235, XL765, pazopanib, cediranib, and axitinib, by providing a subject sample from the subject, determining the level of hypoxia in a tumor from the subject in vitro, wherein a high level of hypoxia in the sample indicates the subject is likely to respond to therapy with an agent selected from the group consisting of bevacizumab, ganetespib, temsirolimus, erlotinib, PTK787, BEZ235, XL765, pazopanib, cediranib, and axitinib.

In certain embodiments, a subject having a low level of hypoxia in the tumor is not likely to respond to therapy with an agent selected from the group consisting of bevacizumab, ganetespib, temsirolimus, erlotinib, PTK787, BEZ235, XL765, pazopanib, cediranib, and axitinib.

In certain embodiments, the cancer is a solid tumor. In certain embodiments, the cancer is a blood tumor, i.e., not a solid tumor. The type of cancer includes, but is not limited to, one or more of the cancer types provided herein.

In certain embodiments, the level of hypoxia in a tumor is determined in a subject sample. The subject sample can include, but is not limited to, one or more of tumor tissue, blood, urine, stool, lymph, cerebrospinal fluid, circulating tumor cells, bronchial lavage, peritoneal lavage, exudate, effusion, and sputum. In certain embodiments, the tumor tissue is tumor tissue that is in the subject or that is removed from the subject.

In certain embodiments, the level of hypoxia is determined by detecting the activity level or expression level of one or more hypoxia modulated polypeptides. In certain embodiments, the activity level or expression level of the one or more hypoxia modulated polypeptides are up regulated in the sample. The level of hypoxia can be determined by any method known in the art including, but not limited to, detecting the activity level or expression level of one or more hypoxia modulated polypeptides or using detection methods selected from the group consisting of detection of activity or expression of at least one isoform or subunit of lactate dehydrogenase (LDH), at least one isoform or subunit of hypoxia inducible factor (HIF), at least one pro-angiogenic form of vascular endothelial growth factor (VEGF), phosphorylated VEGF receptor (pKDR) 1, 2, and 3; neurolipin 1 (NRP-1), pyruvate dehydrokinase (PDH-K), ornithine decarboxylase (ODC), glucose transporter-1 (GLUT-1), glucose transporter-2 (GLUT-2), tumor size, blood flow, EF5 binding, pimonidazole binding, PET scan, and probe detection of hypoxia level.

In certain embodiments, the isoform or subunit of LDH comprises one or more selected from the group consisting of, LDH5, LDH4, LDH3, LDH2, LDH1, LDHA and LDHB; or any combination thereof including total LDH. In certain embodiments, the isoform of HIF comprises one or more selected from the group consisting of HIF-1α, HIF-1β, HIF-2α, and HIF-2β; or any combination thereof including total HIF-1 and/or HIF-2. In certain embodiments, the pro-angiogenic isoform of VEGF is any VEGF-A isoform, or any combination of VEGF-A isoforms including total VEGF-A.

In certain embodiments, detection of a high level of activity or expression of at least one LDH isoform or subunit comprises detection of an LDH activity or expression level of an LDH selected from the group consisting of total LDH, LDH5, LDH4, LDH5 plus LDH4, LDH5 plus LDH4 plus LDH3, and LDHA, wherein the activity level or expression level is 0.8 ULN or more. In certain embodiments, detection of a high level of activity or expression of at least one LDH isoform or subunit comprises detection of an LDH activity or expression level of an LDH selected from the group consisting of total LDH, LDH5, LDH4, LDH5 plus LDH4, LDH5 plus LDH4 plus LDH3, and LDHA, wherein the activity level or expression level is 1.0 ULN or more.

In certain embodiments, detection of a high level of hypoxia comprises detection of a change in a ratio or levels of activity or expression or a change in a ratio of normalized levels of activity or expression of hypoxia modulated polypeptides. In certain embodiments, a high level of hypoxia comprises a ratio or a normalized ratio of 1.0 or more of the ULN, wherein the ratio or normalized ratio is selected from the group consisting of the LDHA to LDHB, LDH5 or LDH4 to LDH1, LDH5 or LDH4 to total LDH, LDH5 and LDH4 to LDH1, LDH5 and LDH4 to total LDH, LDH5, LDH4, and LDH3 to LDH1, and LDH5, LDH4, and LDH3 to total LDH.

In certain embodiments, the subject was previously treated with another chemotherapeutic agent.

The invention further provides kits to practice the methods or uses of diagnosis, treatment, or any other method or use provided herein.

In certain embodiments, a kit includes at least one of bevacizumab, ganetespib, temsirolimus, erlotinib, sorafenib, sunitinib, PTK787, BEZ235, XL765, pazopanib, cediranib, and axitinib and instruction for administration of bevacizumab, ganetespib, temsirolimus, erlotinib, PTK787, BEZ235, XL765, pazopanib, cediranib, and axitinib to a subject having a tumor with a high level of hypoxia.

In certain embodiments, the kit includes at least one reagent specifically for detection of a level of hypoxia and instructions for administering at least one of bevacizumab, ganetespib, temsirolimus, erlotinib, sorafenib, sunitinib, PTK787, BEZ235, XL765, pazopanib, cediranib, and axitinib to a subject with cancer identified as having a high level of hypoxia. It is understood that not all of the components of the kit need to be in a single package.

In certain embodiments of the invention, the agent comprises bevacizumab.

In certain embodiments of the invention, the agent comprises ganetespib.

In certain embodiments of the invention, the agent comprises temsirolimus.

In certain embodiments of the invention, the agent comprises erlotinib.

In certain embodiments of the invention, the agent comprises PTK787.

In certain embodiments of the invention, the agent comprises BEZ235.

In certain embodiments of the invention, the agent comprises XL765.

In certain embodiments of the invention, the agent comprises pazopanib.

In certain embodiments of the invention, the agent comprises cediranib.

In certain embodiments of the invention, the agent comprises axitinib.

Other embodiments of the invention are provided infra.

### BRIEF DESCRIPTION OF THE DRAWINGS

FIGS. 1A and B show the activity of LDH5 as a percent of total LDH activity in serum samples from nude mice with (A) HCT116 tumors or (B) 786-O tumors relative to tumor volume. FIGS. 1C and D show the protein levels of LDH5 as a percent of total LDH activity in serum samples from nude mice with (C) HCT116 tumors or (D) 786-O tumors relative to tumor volume.

FIG. 2A shows the results from a study examining bevacizumab single agent activity dosed at 1×/week i.p. in the HCT116 human colon carcinoma xenograft model in nude mice. % T/C (treatment/control) values for day 38 are indicated on the right.

FIG. 2B shows the results from a study examining bevacizumab single agent activity dosed at 1×/week i.p. in the 786-O human renal carcinoma xenograft model in nude mice. % T/C values for day 34 are indicated on the right.

FIG. 3A shows the results from a study examining vatalanib single agent activity dosed at 5×/week p.o. in the HCT116 human colon carcinoma xenograft model in nude mice. % T/C (treatment/control) values for day 38 are indicated on the right.

FIG. 3B shows the results from a study examining vatalanib single agent activity dosed at 5×/week p.o. in the 786-O human renal carcinoma xenograft model in nude mice. % T/C values for day 34 are indicated on the right.

FIG. 4A shows the results from a study examining XL765 single agent activity dosed at 5×/week p.o. in the HCT116 human colon carcinoma xenograft model in nude mice. % T/C values for day 39 are indicated on the right.

FIG. 4B shows the results from a study examining XL765 single agent activity dosed at 5×/week p.o. in the 786-O human renal carcinoma xenograft model in nude mice. % T/C values for day 35 are indicated on the right.

FIG. 5A shows the results from a study examining erlotinib single agent activity dosed at 1×/week p.o. in the HCT116 human colon carcinoma xenograft model in nude mice. % T/C values for day 39 are indicated on the right.

FIG. 5B shows the results from a study examining erlotinib single agent activity dosed at 1×/week p.o. in the 786-O human colon carcinoma xenograft model in nude mice. % T/C values for day 39 are indicated on the right.

### DETAILED DESCRIPTION OF THE INVENTION

Research has provided the physician with ever more options for therapeutics for the treatment of cancer. However, despite the availability of the new agents, the ability to match a therapeutic agent to a specific patient based not just on the type of tumor or site of the tumor, but the characteristic of the tumor, is lacking. The instant invention provides methods of identifying a subject who will likely respond favorably to treatment with a selected agent by determining the level of hypoxia in a tumor, either by looking directly at markers within the tumor tissue or looking at markers in a peripheral sample from the subject, e.g., a bodily fluid such as blood, serum, plasma, lymph, urine, cerebrospinal fluid, fecal matter, circulating tumor cells, bronchial lavage, peritoneal lavage, exudate, effusion, and sputum for the presence of one or more indicators of the level of hypoxia in the tumor.

In order that the present invention may be more readily understood, certain terms are first defined. In addition, it should be noted that whenever a value or range of values of a parameter are recited, it is intended that values and ranges intermediate to the recited values are also intended to be part of this invention.

### I. DEFINITIONS

The articles “a”, “an” and “the” are used herein to refer to one or to more than one (i.e. to at least one) of the grammatical object of the article unless otherwise clearly indicated by contrast. By way of example, “an element” means one element or more than one element.

The term “including” is used herein to mean, and is used interchangeably with, the phrase “including but not limited to”.

The term “or” is used herein to mean, and is used interchangeably with, the term “and/or,” unless context clearly indicates otherwise.

The term “such as” is used herein to mean, and is used interchangeably, with the phrase “such as but not limited to”.

Unless specifically stated or obvious from context, as used herein, the term “about” is understood as within a range of normal tolerance in the art, for example within 2 standard deviations of the mean. About can be understood as within 10%, 9%, 8%, 7%, 6%, 5%, 4%, 3%, 2%, 1%, 0.5%, 0.1%, 0.05%, or 0.01% of the stated value. Unless otherwise clear from context, all numerical values provided herein can be modified by the term about.

The recitation of a listing of chemical group(s) in any definition of a variable herein includes definitions of that variable as any single group or combination of listed groups. The recitation of an embodiment for a variable or aspect herein includes that embodiment as any single embodiment or in combination with any other embodiments or portions thereof.

Any compositions or methods provided herein can be combined with one or more of any of the other compositions and methods provided herein.

As used herein, the term “subject” refers to human and non-human animals, including veterinary subjects. The term “non-human animal” includes all vertebrates, e.g., mammals and non-mammals, such as non-human primates, mice, rabbits, sheep, dog, cat, horse, cow, chickens, amphibians, and reptiles. In a preferred embodiment, the subject is a human and may be referred to as a patient.

As used herein, the terms “treat,” “treating” or “treatment” refer, preferably, to an action to obtain a beneficial or desired clinical result including, but not limited to, alleviation or amelioration of one or more signs or symptoms of a disease or condition, diminishing the extent of disease, stability (i.e., not worsening) state of disease, amelioration or palliation of the disease state, diminishing rate of or time to progression, and remission (whether partial or total), whether detectable or undetectable. “Treatment” can also mean prolonging survival as compared to expected survival in the absence of treatment. Treatment does not need to be curative.

A “therapeutically effective amount” is that amount sufficient to treat a disease in a subject. A therapeutically effective amount can be administered in one or more administrations.

By “diagnosing” and the like, as used herein, refers to a clinical or other assessment of the condition of a subject based on observation, testing, or circumstances for identifying a subject having a disease, disorder, or condition based on the presence of at least one indicator, such as a sign or symptom of the disease, disorder, or condition. Typically, diagnosing using the method of the invention includes the observation of the subject for multiple indicators of the disease, disorder, or condition in conjunction with the methods provided herein. Diagnostic methods provide an indicator that a disease is or is not present. A single diagnostic test typically does not provide a definitive conclusion regarding the disease state of the subject being tested.

The terms “administer”, “administering” or “administration” include any method of delivery of a pharmaceutical composition or agent into a subject's system or to a particular region in or on a subject. In certain embodiments of the invention, an agent is administered intravenously, intramuscularly, subcutaneously, intradermally, intranasally, orally, transcutaneously, or mucosally. In a preferred embodiment, an agent is administered intravenously. Administering an agent can be performed by a number of people working in concert. Administering an agent includes, for example, prescribing an agent to be administered to a subject and/or providing instructions, directly or through another, to take a specific agent, either by self-delivery, e.g., as by oral delivery, subcutaneous delivery, intravenous delivery through a central line, etc.; or for delivery by a trained professional, e.g., intravenous delivery, intramuscular delivery, intratumoral delivery, etc.

As used herein, the term “survival” refers to the continuation of life of a subject which has been treated for a disease or condition, e.g., cancer.

As used herein, the term “recur” refers to the re-growth of tumor or cancerous cells in a subject in whom primary treatment for the tumor has been administered. The tumor may recur in the original site or in another part of the body. In one embodiment, a tumor that recurs is of the same type as the original tumor for which the subject was treated. For example, if a subject had an ovarian cancer tumor, was treated and subsequently developed another ovarian cancer tumor, the tumor has recurred. In addition, a cancer can recur in or metastasize to a different organ or tissue than the one where it originally occurred.

As used herein, the terms “identify” or “select” refer to a choice in preference to another. In other words, to identify a subject or select a subject is to perform the active step of picking out that particular subject from a group and confirming the identity of the subject by name or other distinguishing feature. With respect to the instant invention, it is understood that identifying a subject or selecting a subject as having a specific level of hypoxia or a specific level of LDH can include any of a number of acts including, but not limited to, performing a test and observing a result that is indicative of a subject having a specific level of hypoxia; reviewing a test result of a subject and identifying the subject as having a specific level of hypoxia; reviewing documentation on a subject stating that the subject has a specific level of hypoxia and identifying the subject as the one discussed in the documentation by confirming the identity of the subject e.g., by an identification card, hospital bracelet, asking the subject for his/her name and/or other personal information to confirm the subjects identity.

As used herein, the term “benefit” refers to something that is advantageous or good, or an advantage. Similarly, the term “benefiting”, as used herein, refers to something that improves or advantages. For example, a subject will benefit from treatment if they exhibit a decrease in at least one sign or symptom of a disease or condition (e.g., tumor shrinkage, decrease in tumor burden, inhibition or decrease of metastasis, improving quality of life (“QOL”), if there is a delay of time to progression (“TTP”), if there is an increase of overall survival (“OS”), etc.), or if there is a slowing or stopping of disease progression (e.g., halting tumor growth or metastasis, or slowing the rate of tumor growth or metastasis). A benefit can also include an improvement in quality of life, or an increase in survival time or progression free survival.

The terms “cancer” or “tumor” are well known in the art and refer to the presence, e.g., in a subject, of cells possessing characteristics typical of cancer-causing cells, such as uncontrolled proliferation, immortality, metastatic potential, rapid growth and proliferation rate, decreased cell death/apoptosis, and certain characteristic morphological features. Cancer cells are often in the form of a solid tumor. However, cancer also includes non-solid tumors, e.g., blood tumors, e.g., leukemia, wherein the cancer cells are derived from bone marrow. As used herein, the term “cancer” includes pre-malignant as well as malignant cancers. Cancers include, but are not limited to, acoustic neuroma, acute leukemia, acute lymphocytic leukemia, acute myelocytic leukemia (monocytic, myeloblastic, adenocarcinoma, angiosarcoma, astrocytoma, myelomonocytic and promyelocytic), acute T-cell leukemia, basal cell carcinoma, bile duct carcinoma, bladder cancer, brain cancer, breast cancer, bronchogenic carcinoma, cervical cancer, chondrosarcoma, chordoma, choriocarcinoma, chronic leukemia, chronic lymphocytic leukemia, chronic myelocytic (granulocytic) leukemia, chronic myelogenous leukemia, colon cancer, colorectal cancer, craniopharyngioma, cystadenocarcinoma, diffuse large B-cell lymphoma, Burkitt's lymphoma, dysproliferative changes (dysplasias and metaplasias), embryonal carcinoma, endometrial cancer, endotheliosarcoma, ependymoma, epithelial carcinoma, erythroleukemia, esophageal cancer, estrogen-receptor positive breast cancer, essential thrombocythemia, Ewing's tumor, fibrosarcoma, follicular lymphoma, germ cell testicular cancer, glioma, heavy chain disease, hemangioblastoma, hepatoma, hepatocellular cancer, hormone insensitive prostate cancer, leiomyosarcoma, liposarcoma, lung cancer, lymphagioendotheliosarcoma, lymphangiosarcoma, lymphoblastic leukemia, lymphoma (Hodgkin's and non-Hodgkin's), malignancies and hyperproliferative disorders of the bladder, breast, colon, lung, ovaries, pancreas, prostate, skin, and uterus, lymphoid malignancies of T-cell or B-cell origin, leukemia, lymphoma, medullary carcinoma, medulloblastoma, melanoma, meningioma, mesothelioma, multiple myeloma, myelogenous leukemia, myeloma, myxosarcoma, neuroblastoma, non-small cell lung cancer, oligodendroglioma, oral cancer, osteogenic sarcoma, ovarian cancer, pancreatic cancer, papillary adenocarcinomas, papillary carcinoma, pinealoma, polycythemia vera, prostate cancer, rectal cancer, renal cell carcinoma, retinoblastoma, rhabdomyosarcoma, sarcoma, sebaceous gland carcinoma, seminoma, skin cancer, small cell lung carcinoma, solid tumors (carcinomas and sarcomas), small cell lung cancer, stomach cancer, squamous cell carcinoma, synovioma, sweat gland carcinoma, thyroid cancer, Waldenstrom's macroglobulinemia, testicular tumors, uterine cancer, and Wilms' tumor. Other cancers include primary cancer, metastatic cancer, oropharyngeal cancer, hypopharyngeal cancer, liver cancer, gall bladder cancer, bile duct cancer, small intestine cancer, urinary tract cancer, kidney cancer, urothelium cancer, female genital tract cancer, uterine cancer, gestational trophoblastic disease, male genital tract cancer, seminal vesicle cancer, testicular cancer, germ cell tumors, endocrine gland tumors, thyroid cancer, adrenal cancer, pituitary gland cancer, hemangioma, sarcoma arising from bone and soft tissues, Kaposi's sarcoma, nerve cancer, ocular cancer, meningial cancer, glioblastomas, neuromas, neuroblastomas, Schwannomas, solid tumors arising from hematopoietic malignancies such as leukemias, metastatic melanoma, recurrent or persistent ovarian epithelial cancer, fallopian tube cancer, primary peritoneal cancer, gastrointestinal stromal tumors, colorectal cancer, gastric cancer, melanoma, glioblastoma multiforme, non-squamous non-small-cell lung cancer, malignant glioma, epithelial ovarian cancer, primary peritoneal serous cancer, metastatic liver cancer, neuroendocrine carcinoma, refractory malignancy, triple negative breast cancer, HER2 amplified breast cancer, nasopharageal cancer, oral cancer, biliary tract, hepatocellular carcinoma, squamous cell carcinomas of the head and neck (SCCHN), non-medullary thyroid carcinoma, recurrent glioblastoma multiforme, neurofibromatosis type 1, CNS cancer, liposarcoma, leiomyosarcoma, salivary gland cancer, mucosal melanoma, acral/lentiginous melanoma, paraganglioma, pheochromocytoma, advanced metastatic cancer, solid tumor, triple negative breast cancer, colorectal cancer, sarcoma, melanoma, renal carcinoma, endometrial cancer, thyroid cancer, rhabdomysarcoma, multiple myeloma, ovarian cancer, glioblastoma, gastrointestinal stromal tumor, mantle cell lymphoma, and refractory malignancy.

“Solid tumor,” as used herein, is understood as any pathogenic tumor that can be palpated or detected using imaging methods as an abnormal growth having three dimensions. A solid tumor is differentiated from a blood tumor such as leukemia. However, cells of a blood tumor are derived from bone marrow, therefore, the tissue producing the cancer cells is a solid tissue that can be hypoxic.

“Tumor tissue” is understood as cells, extracellular matrix, and other naturally occurring components associated with the solid tumor.

As used herein, the term “isolated” refers to a preparation that is substantially free (e.g., 50%, 60%, 70%, 80%, 90% or more, by weight) from other proteins, nucleic acids, or compounds associated with the tissue from which the preparation is obtained.

The term “sample” as used herein refers to a collection of similar fluids, cells, or tissues isolated from a subject. The term “sample” includes any body fluid (e.g., urine, serum, blood fluids, lymph, gynecological fluids, cystic fluid, ascetic fluid, ocular fluids, and fluids collected by bronchial lavage and/or peritoneal rinsing), ascites, tissue samples (e.g., tumor samples) or a cell from a subject. Other subject samples include tear drops, serum, cerebrospinal fluid, feces, sputum, and cell extracts. In one embodiment, the sample is removed from the subject. In a particular embodiment, the sample is urine or serum. In another embodiment, the sample does not include ascites or is not an ascites sample. In another embodiment, the sample does not include peritoneal fluid or is not peritoneal fluid. In one embodiment, the sample comprises cells. In another embodiment, the sample does not comprise cells. In certain embodiments, the sample can be the portion of the subject that is imaged (e.g., using a PET scan, a functional imaging method such as MRI to detect blood flow) or tested to determine level of hypoxia (e.g., tumor tissue assayed for level of hypoxia using a probe). Samples are typically removed from the subject prior to analysis, however, tumor samples can be analyzed in the subject, for example, using imaging or other detection methods.

In some embodiments, only a portion of the sample is subjected to an assay for determining the level of hypoxia or the level of the tumor using any method provided herein. In certain embodiments, the level of hypoxia is indicated by the level of an isoform or subunit of lactate dehydrogenase (LDH) or any combination of subunits or isoforms including total LDH, or various portions of the sample are subjected to various assays for determining the level of hypoxia or the level of an isoform or subunit of LDH. Also, in many embodiments, the sample may be pre-treated by physical or chemical means prior to the assay. For example, samples, for example, blood samples, can be subjected to centrifugation, dilution and/or treatment with a solubilizing substance prior to assaying the samples for the level of hypoxia or LDH. Such techniques serve to enhance the accuracy, reliability and reproducibility of the assays of the present invention.

The term “control sample,” as used herein, refers to any clinically relevant comparative sample, including, for example, a sample from a healthy subject not afflicted with cancer, a sample from a subject having a less severe or slower progressing cancer than the subject to be assessed, a sample from a subject having some other type of cancer or disease, a sample from a subject prior to treatment, a sample of non-diseased tissue (e.g., non-tumor tissue), a sample from the same origin and close to the tumor site, and the like. A control sample can be a purified sample, protein, and/or nucleic acid provided with a kit. Such control samples can be diluted, for example, in a dilution series to allow for quantitative measurement of analytes in test samples. A control sample may include a sample derived from one or more subjects. A control sample may also be a sample made at an earlier time point from the subject to be assessed. For example, the control sample could be a sample taken from the subject to be assessed before the onset of the cancer, at an earlier stage of disease, or before the administration of treatment or of a portion of treatment. The control sample may also be a sample from an animal model, or from a tissue or cell lines derived from the animal model, of the cancer. The level of LDH in a control sample that consists of a group of measurements may be determined, e.g., based on any appropriate statistical measure, such as, for example, measures of central tendency including average, median, or modal values.

The term “control level” refers to an accepted or pre-determined level of hypoxia or LDH which is used to compare with the level of hypoxia or LDH in a sample derived from a subject. For example, in one embodiment, the control level of hypoxia is based on the level of hypoxia in sample(s) from a subject(s) having slow disease progression. In another embodiment, the control level of hypoxia is based on the level in a sample from a subject(s) having rapid disease progression. In another embodiment, the control level of hypoxia is based on the level of hypoxia in a sample(s) from an unaffected, i.e., non-diseased, subject(s), i.e., a subject who does not have cancer. In yet another embodiment, the control level of hypoxia is based on the level of hypoxia in a sample from a subject(s) prior to the administration of a therapy for cancer. In another embodiment, the control level of hypoxia is based on the level of hypoxia in a sample(s) from a subject(s) having cancer that is not contacted with a test compound. In another embodiment, the control level of hypoxia is based on the level of hypoxia in a sample(s) from a subject(s) not having cancer that is contacted with a test compound. In one embodiment, the control level of hypoxia is based on the level of hypoxia in a sample(s) from an animal model of cancer, a cell, or a cell line derived from the animal model of cancer. In another embodiment, the control level of hypoxia is listed in a chart.

In one embodiment, the control is a standardized control, such as, for example, a control which is predetermined using an average of the levels of hypoxia from a population of subjects having no cancer. In still other embodiments of the invention, a control level of hypoxia is based on the level of hypoxia in a non-cancerous sample(s) derived from the subject having cancer. For example, when a biopsy or other medical procedure reveals the presence of cancer in one portion of the tissue, the control level of hypoxia may be determined using the non-affected portion of the tissue, and this control level may be compared with the level of hypoxia in an affected portion of the tissue. Similarly, when a biopsy or other medical procedure reveals the presence of a cancer in one portion of the tissue, the control level of hypoxia may be determined using the non-affected portion of the tissue, and this control level may be compared with the level of hypoxia in an affected portion of the tissue.

As used herein, the term “obtaining” is understood herein as manufacturing, purchasing, or otherwise coming into possession of.

As used herein, the term “lactate dehydrogenase” refers to an enzyme that interconverts pyruvate and lactate with concomitant interconversion of NADH and NAD+. Under conditions of hypoxia, the reaction favors the conversion of pyruvate to lactate. Under conditions of normoxia, or low levels of hypoxia, the reaction favors the conversion of lactate to pyruvate. Functional lactate dehydrogenase are homo or hetero tetramers composed of M and H protein subunits encoded by the LDHA and LDHB genes respectively: LDH-1 (4H) is the predominant form found, for example, in the heart and red blood cells (RBCs); LDH-2 (3H1M) is the predominant found, for example, in the reticuloendothelial system; LDH-3 (2H2M) is the predominant form found, for example, in the lungs; LDH-4 (1H3M) is the predominant form found, for example, in the kidneys, placenta and pancreas; and LDH-5 (4M) is the predominant form found, for example, in the liver and striated muscle. Typically, multiple forms of LDH are found in these tissues. Lactate dehydrogenase is classified as (EC 1.1.1.27). The specific ratios tested may be tumor-type specific.

As used herein, the terms “hypoxia” and “hypoxic” refer to a condition in which a cancer or a tumor has a low oxygen microenvironment or a less well-oxygenated microenvironment. Hypoxia occurs when tumor growth exceeds new blood vessel formation, and a tumor must undergo genetic and adaptive changes to allow them to survive and proliferate in the hypoxic environment. The development of intratumoral hypoxia is a common sign of solid tumors. When a tumor microenvironment is less well-oxygenated, there is a greater dependency on oxygen-sensitive pathways, including but not limited to HIF1α pathways, VEGF pathways, and mTOR pathways. These pathways facilitate crucial adaptive mechanisms, such as angiogenesis, glycolysis, growth-factor signaling, immortalization, genetic instability, tissue invasion and metastasis, apoptosis, and pH regulation (see, e.g., Harris, Nature Reviews, 2:38-47, 2002). These pathways may also facilitate invasion and metastasis. Accordingly, the treatment of a subject with a cancer or tumor with a selected agent such as bevacizumab, ganetespib, temsirolimus, erlotinib, PTK787, BEZ235, XL765, pazopanib, cediranib, or axitinib is more effective when the subject has a tumor that exhibits a modulated level of hypoxia, e.g., a high level of hypoxia. As the level of hypoxia in the tumor can be determined by obtaining a sample from a site other than the tumor, as used herein, the subject can be stated to demonstrate a modulated level of hypoxia when it is the tumor present in the subject that demonstrates a modulated level of hypoxia. As used herein it is understood that the subject with a modulated level of hypoxia is typically not suffering from systemic oxygen imbalance or ischemic disease at a site remote from the tumor.

As used herein, the term “level of hypoxia” is understood as the amount of one or more markers indicative of a low oxygen level, or cells having characteristics and/or employing biological pathways characteristic of cells with a low oxygen level, e.g., due to the Warburg effect. Such markers include, but are not limited to, lactate dehydrogenase (LDH), at least one isoform or subunit of hypoxia inducible factor (HIF), at least one pro-angiogenic form of vascular endothelial growth factor (VEGF), phosphorylated VEGF receptor (pKDR) 1, 2, or 3; neurolipin 1 (NRP-1), pyruvate dehydrokinase (PDH-K), and ornithine decarboxylase (ODC). Tumor size can also be correlated with a level of hypoxia. A level of hypoxia can also be determined by PET scan. LDH can be one or more isoforms or subunits of LDH such as LDH5, LDH4, LDH3, LDH2, LDH1, LDHM (also known as LDHA) and LDHH (also known as LDHB). In one embodiment, LDH can be a total sample of all LDH isoforms or subunits. “Hypoxia inducible factors” or “HIFs” are transcription factors which respond to changes in available oxygen in a cellular environment. HIF1α is a master regulator of hypoxic gene expression and oxygen homeostasis. HIF can be one or more subunits or isoforms of HIF including HIF-1α, HIF-1β, HIF-2α, and HIF-2β. VEGF can be one or more of the various splice forms of VEGF including pro-angiogenic VEGF-A and antiangiogenic VEGF-B.

As used herein, the term “level of LDH” refers to the amount of LDH present in a sample which can be used to indicate the presence or absence of hypoxia in the tumor in the subject from whom the sample was obtained. LDH enables the conversion of pyruvate to lactate and is a critical component of glycolysis under hypoxic conditions. LDH can be total LDH or one or more isoforms or subunits of LDH such as LDH5, LDH4, LDH3, LDH2, LDH1, LDHM (also known as LDHA) and LDHH (also known as LDHB). A modulated level of LDH can refer to a high level of LDH or a low level of LDH. In one embodiment, a PET scan (which is positive when aerobic glycolysis is active) is an indicator of a high level of LDH. In another embodiment, a PET scan (which is negative when aerobic glycolysis is inactive) is an indicator of a low level of LDH. In one embodiment, a high level of LDH is at least 1.1, 1.2, 1.3, 1.4, 1.5, 1.6, 1.7, 1.8, 1.9, 2.0, 2.1, 2.2, 2.3, 2.4, 2.5, 2.6, 2.7, 2.8, 2.9, 3, 4, 5, 6, 7, 8, 9, or 10 times the value of normal level of LDH. In another embodiment, a low level of LDH is 0.9, 0.8, 0.7, 0.6, 0.5, 0.4, 0.3, 0.2, or 0.1 times the value of a normal level of LDH. A normal level of LDH, or any other marker, can be defined as any value within the range of normal, or the upper limit of the normal value, or the lower limit of the normal value. Assays for determining the level of LDH in a sample are well known in the art and provided herein.

In another embodiment, the level of LDH can be understood to be a change in the relative levels of protein or activity of LDH isoforms or the ratio of LDH isoforms. Preferably, the ratios are the ratios of normalized values, e.g., the level of the LDH subunit or isoform is normalized to the ULN, the LLN, or a median value. A change of the relative levels of the isoforms can be indicative of the level of hypoxia. For example, an increase in the level of LDHA relative to LDHB can be indicative of an increase in hypoxia. Alternatively, an increase in the level of LDH5 and/or LDH4, either individually or in total, relative to the level of LDH1 or total LDH can be indicative of an increase in hypoxia. The relative levels can be compared to relative levels in an appropriate control sample from normal subjects, e.g., subjects without cancer or ischemic disease. That is, the ratios are the ratios of normalized values, e.g., the level of the LDH subunit or isoform is normalized to the ULN, the LLN, or a median value. The normal levels can be considered to be a range with an upper level of normal and a lower level of normal. In certain embodiments, a high level of LDH can be understood an increase in the normalized level of LDHA or LDH5 and/or LDH4 relative to the normalized level of LDHB or LDH1 or total LDH, respectively, or to total LDH of at least 1.1, 1.2, 1.3, 1.4, 1.5, 1.6, 1.7, 1.8, 1.9, 2.0, 2.1, 2.2, 2.3, 2.4, 2.5, 2.6, 2.7, 2.8, 2.9, 3, 4, 5, 6, 7, 8, 9, or 10 times the value of normalized level of LDHA or LDH5 and/or LDH4 relative to the normalized level of LDHB or LDH1 or total LDH, respectively. In another embodiment, a low level of LDH is a ratio of 0.9, 0.8, 0.7, 0.6, 0.5, 0.4, 0.3, 0.2, or 0.1 of the normalized value of LDHA or LDH5 and/or LDH4 relative to the normalized level of LDHB or LDH1 or total LDH, respectively.

As used herein, a “normalized ratio” is understood as a proportion of two values that have been compared to a standard, either an external (e.g., population control level) or an internal (e.g., level from a normal tissue, level from an earlier time point, level of one or more isoforms) control to allow for comparison of samples between individuals. For example, the ratio of normalized levels of hypoxia modulated polypeptides can be determined by determining a ratio of two normalized levels of two isoforms or subunits of LDH or total LDH by comparing the level of a first isoform or subunit of LDH in the sample relative to a control sample to provide a first normalized level, and the level of a second isoform or subunit of LDH or total LDH relative to a control sample to provide a second normalized level, and calculating a ratio of the first normalized level and the second normalized level to provide a normalized ratio of LDH isoforms or subunits, wherein at least one of the first level and the second level are not total LDH. In certain embodiments, a low level of hypoxia is a normalized ratio of the ULN of LDHA to LDHB of 1.0 or less, or a normalized ratio of the ULN of LDH5 and/or LDH4 to LDH1 or total LDH of 1.0 or less.

Assays for determining the level of LDH in a sample are well known in the art. See, e.g., U.S. Publication Nos. 2010/0178283 and 2008/0213744 and U.S. Pat. Nos. 4,250,255 and 6,242,208, the entire contents of each of which are expressly incorporated herein by reference. LDH sequences are further provided in public databases (e.g., at blast.ncbi.nlm.nih.gov/Blast.cgi).

It is also understood that levels of the various markers can include the level of a post-translationally modified marker, e.g., the total amount of an isoform of HIF may remain the same, but the amount of the hydroxylated version of the HIF may increase. In addition, it is noted that HIF and other hypoxia modulated polypeptides can be upregulated by a number of conditions other than hypoxia, e.g., pH change, changes in levels of O₂.* or H₂O₂, etc. Accordingly, although the term “level of expression,” as used herein, is intended to encompass all hypoxia responsive factors, a change in their level of expression may or may not actually directly reflect the amount of oxygen available to the tumor.

Methods to detect the levels of markers of hypoxia are well known in the art.

Antibodies against and kits for detection of hypoxia modulated polypeptides can be purchased from a number of commercial sources. Alternatively, using routine methods known in the art (e.g., immunization of animals, phage display, etc.) antibodies against one or more hypoxia modulated polypeptides or subunits or isoforms thereof can be made and characterized. Antibodies can be used for the detection of levels of hypoxia using ELISA, RIA, or other immunoassay methods, preferably automated methods, for the quantitative detection of proteins in samples of bodily fluids or homogenized solid samples. Hypoxia can be detected by enzyme activity assays (e.g., LDH activity, kinase activity) including in gel assays to resolve the activity of various isoforms of proteins. Alternatively, immunohistochemical methods can be used on tumor samples and tissue sections. Antibodies against prodrugs that localize in hypoxic regions (e.g., EF5, pimonidazole, etc.) can also be used to detect hypoxia. Functional imaging measuring blood flow in the tumor can be used as an indicator of hypoxia in the tissue. Direct measurement of hypoxia can be preformed by inserting a sensor into the tumor. Qualitative scoring methods and scanning methods to detect staining are known in the art. When qualitative scoring methods are used, it is preferred that two independent, blinded technicians, pathologists, or other skilled individuals analyze each sample with specific methods for resolving any significant disagreement in scoring, e.g., a third individual reviews the tissue sample.

Alternatively, nucleic acid-based methods of detection of levels of hypoxia are also well known in the art. Methods of designing primers and probes for quantitative reverse transcription real time (rt) PCR are known in the art. Methods for performing northern blots to detect RNA levels are known in the art. Nucleic acid detection methods can also include fluorescence in situ hybridization (FISH) and in situ PCR. Qualitative scoring methods and scanning methods to detect staining are known in the art. When qualitative scoring methods are used, it is preferred that two independent, blinded technicians, pathologists, or other skilled individuals analyze each sample with specific methods for resolving any significant disagreement in scoring, e.g., a third individual reviews the tissue sample.

“Baseline” refers to the level of hypoxia or the level of LDH upon patient entrance into the study and is used to distinguish from levels of hypoxia or levels of LDH the patient might have during or after treatment.

“Elevated” or “lower” refers to a patient's value relative to the upper limit of normal (“ULN”) or the lower limit of normal (“LLN”) which are based on historical normal control samples. As the level of the hypoxic marker present in the subject will be a result of the disease, and not a result of treatment, typically not a control, a sample obtained from the patient prior to onset of the disease will not likely be available. Because different labs may have different absolute results, LDH values are presented relative to that lab's upper limit of normal value (ULN). LDH can be expressed in IU/ml (International Units per milliliter). An accepted ULN for LDH is 234 IU/ml, however, this value is not universally accepted or applicable to all methods of detection of LDH in all samples.

The specific value for ULN and LLN will also depend, for example, on the type of assay (e.g., ELISA, enzyme activity, immunohistochemistry, imaging), the sample to be tested (e.g., serum, tumor tissue, urine), and other considerations known to those of skill in the art. The ULN or LLN can be used to define cut-offs between normal and abnormal. For example, a low level of a marker (e.g., LDH) can be defined as a marker level less than or equal to the ULN for that marker, with a high level being all values greater than the ULN. Cut-offs can also be defined as fractional amounts of the ULN. For example, a low level of a marker can be understood to be a level of about 0.5 ULN or less, 0.6 ULN or less, 0.7 ULN or less, 0.8 ULN or less, 0.9 ULN or less, 1.0 ULN or less, 1.1 ULN or less, 1.2 ULN or less, 1.3 ULN or less, 1.4 ULN or less, 1.5 ULN or less, 1.6 ULN or less, 1.7 ULN or less, 1.8 ULN or less, 1.9 ULN or less, 2.0 ULN or less, 2.5 ULN or less, 3.0 ULN or less, or 4.0 ULN or less, with the corresponding high level of the marker being a value greater than the low level. In certain embodiments, the presence of a low level of a marker in a subject sample as defined above can be indicative that a subject will or will not respond to a particular therapeutic intervention. In certain embodiments, the presence of a high level of a marker in a subject sample as defined above can be indicative that a subject will or will not respond to a particular therapeutic intervention.

Marker levels can also be further stratified, for example, into low, intermediate, and high based on the ULN value. For example, the presence of a low level of a marker in a subject sample as defined above can be indicative that a subject will or will not respond to a particular therapeutic intervention. An intermediate level of a marker, e.g., a range bracketed by any range within the values of 0.5 ULN, 0.6 ULN, 0.7 ULN, 0.8 ULN, 0.9 ULN, 1.0 ULN, 1.1 ULN, 1.2 ULN, 1.3 ULN, 1.4 ULN, 1.5 ULN, 1.6 ULN, 1.7 ULN, 1.8 ULN, 1.9 ULN, and 2.0 ULN, can be considered an intermediate range wherein the level of the marker may be indeterminate that a subject will or will not respond to a particular therapeutic intervention. A high level, greater than the intermediate level, would be indicative that a subject will or will not respond to a particular therapeutic intervention.

Similarly, cut-offs of ratios of LDH subunits or isoforms comparing the ULN, the LLN, or the median values to differentiate between high and low levels of hypoxia can be defined as any value or range bracketed by the values 0.5, 0.6, 0.7, 0.8, 0.9, 1.0, 1.1, 1.2, 1.3, 1.4, 1.5, 1.6, 1.7, 1.8, 1.9, 2.0, 2.1, 2.2, 2.3, 2.4, 2.5, 2.6, 2.7, 2.8, 2.9, 3.0, or higher.

The “normal” level of expression of a marker is the level of expression of the marker in cells of a subject or patient not afflicted with cancer. In one embodiment, a “normal” level of expression refers to the level of expression of the marker under normoxic conditions.

An “over-expression” or “high level of expression” of a marker refers to an expression level in a test sample that is greater than the standard error of the assay employed to assess expression, and is preferably at least 1.1, 1.2, 1.3, 1.4, 1.5, 16, 1.7, 1.8, 1.9, 2.0, 2.1, 2.2, 2.3, 2.4, 2.5, 2.6, 2.7, 2.8, 2.9, 3, 4, 5, 6, 7, 8, 9, or 10 times the expression level of the marker in a control sample (e.g., sample from a healthy subject not having the marker associated disease, i.e., cancer). In one embodiment, expression of a marker is compared to an average expression level of the marker in several control samples.

A “low level of expression” or “under-expression” of a marker refers to an expression level in a test sample that is less than at least 0.9, 0.8, 0.7, 0.6, 0.5, 0.4, 0.3, 0.2, or 0.1 times the expression level of the marker in a control sample (e.g., sample from a healthy subjects not having the marker associated disease, i.e., cancer). In one embodiment, expression of a marker is compared to an average expression level of the marker in several control samples.

As used herein, the term “identical” or “identity” is used herein in relation to amino acid or nucleic acid sequences refers to any gene or protein sequence that bears at least 30% identity, more preferably 40%, 50%, 60%, 70%, 75%, 80%, 81%, 82%, 83%, 84%, 85%, 86%, 87%, 88%, 89%, 90%, 91%, 92%, 93%, 94%, and most preferably 95%, 96%, 97%, 98%, 99% or more identity to a known gene or protein sequence over the length of the comparison sequence. Protein or nucleic acid sequences with high levels of identity throughout the sequence can be said to be homologous. A “homologous” protein can also have at least one biological activity of the comparison protein. In general, for proteins, the length of comparison sequences will be at least 10 amino acids, preferably 10, 20, 30, 40, 50, 60, 70, 80, 90, 100, 150, 175, 200, 250, or at least 300 amino acids or more. For nucleic acids, the length of comparison sequences will generally be at least 25, 50, 100, 125, 150, 200, 250, 300, 350, 400, 450, 500, 550, 600, 650, 700, 800, or at least 850 nucleotides or more.

By “hybridize” is meant pair to form a double-stranded molecule between complementary polynucleotide sequences, or portions thereof, under various conditions of stringency. (See, e.g., Wahl and Berger Methods Enzymol. 152:399, 1987; Kimmel, Methods Enzymol. 152:507, 1987.) For example, stringent salt concentration will ordinarily be less than about 750 mM NaCl and 75 mM trisodium citrate, preferably less than about 500 mM NaCl and 50 mM trisodium citrate, and most preferably less than about 250 mM NaCl and 25 mM trisodium citrate. Low stringency hybridization can be obtained in the absence of organic solvent, e.g., formamide, while high stringency hybridization can be obtained in the presence of at least about 35% formamide, and most preferably at least about 50% formamide. Stringent temperature conditions will ordinarily include temperatures of at least about 30° C., more preferably of at least about 37° C., and most preferably of at least about 42° C. Varying additional parameters, such as hybridization time, the concentration of detergent, e.g., sodium dodecyl sulfate (SDS), and the inclusion or exclusion of carrier DNA, are well known to those skilled in the art. Various levels of stringency are accomplished by combining these various conditions as needed. In a preferred embodiment, hybridization will occur at 30° C. in 750 mM NaCl, 75 mM trisodium citrate, and 1% SDS. In a more preferred embodiment, hybridization will occur at 37° C. in 500 mM NaCl, 50 mM trisodium citrate, 1% SDS, 35% formamide, and 100 μg/ml denatured salmon sperm DNA (ssDNA). In a most preferred embodiment, hybridization will occur at 42° C. in 250 mM NaCl, 25 mM trisodium citrate, 1% SDS, 50% formamide, and 200 μg/ml ssDNA. Useful variations on these conditions will be readily apparent to those skilled in the art.

As used herein, the term “oxygen-sensitive pathway” is a cellular signaling pathway which is activated by hypoxia. Oxygen-sensitive pathways may be up-regulated by hypoxia. Alternatively, an oxygen-sensitive pathway may be down-regulated by hypoxia. Oxygen-sensitive pathways include, but are not limited to, HIF pathways (such as HIF1α pathways), VEGF pathways, and mTOR pathways. As used herein, the term “hypoxia-modulated gene” or “hypoxia-modulated polypeptide” refers to a gene or protein which is up-regulated or down-regulated by hypoxia.

As used herein, the term “HIF pathway” and “HIF pathway members” as used herein, describe proteins and other signaling molecules that are regulated by HIF-1 and HIF-2. Hypoxia-Inducible Factor 1 (HIF-1) is a transcription factor that has been shown to play an essential role in cellular responses to hypoxia. Upon hypoxic stimulation, HIF-1 has been shown to activate genes that contain Hypoxic Response Elements (HREs) in their promoters, and thus up-regulate a series of gene products that promote cell survival under conditions of low oxygen availability. The list of known HIF-responsive genes includes glycolytic enzymes (such as lactate dehydrogenase (LDH), enolase-1 (ENO-I), and aldolase A, glucose transporters (GLUT 1 and GLUT 3), vascular endothelial growth factor (VEGF), inducible nitric oxide synthase (NOS-2), and erythropoietin (EPO). The switch of the cell to anaerobic glycolysis, and the up-regulation of angiogenesis by VEGF is geared at maximizing cell survival under conditions of low oxygen tension by reducing the requirement for oxygen, and increasing vasculature to maximize oxygen delivery to tissues. The HIF-1 transcription complex has recently been shown to comprise a heterodimer of two basic helix-loop-helix proteins, HIF-1α and HIF-1β (also known as ARNT, Aryl Hydrocarbon Receptor Nuclear Translocator).

HIF-1α is a member of the basic-helix-loop-helix PAS domain protein family and is an approximately 120 kDa protein containing two transactivation domains (TAD) in its carboxy-terminal half and DNA binding activity located in the N-terminal half of the molecule. HIF-1α is constitutively degraded by the ubiquitin-proteosome pathway under conditions of normoxia, a process that is facilitated by binding of the von Hippel-Lindau (VHL) tumor suppressor protein to HIF-1α. Under conditions of hypoxia, degradation of HIF-1α is blocked and active HIF-1α accumulates. The subsequent dimerization of HIF-1α a with ARNT leads to the formation of active HIF transcription complexes in the nucleus, which can bind to and activate HREs on HIF-responsive genes.

As used herein, the term “VEGF pathway” and “VEGF pathway members” as used herein, describe proteins and other signaling molecules that are regulated by VEGF. For example, VEGF pathway members include VEGFR1, 2, and 3; PECAM-1, LacCer synthase, and PLA2.

As used herein, the term “mTOR pathway” and “mTOR pathway members” as used herein, describe proteins and other signaling molecules that are regulated by mTOR. For example, mTOR pathway members include SK6, PDCD4, eIF4B, RPS6, eIF4, 4E-BP1, and eIF4E.

“Chemotherapeutic agent” is understood as a drug used for the treatment of cancer. Chemotherapeutic agents include, but are not limited to, small molecules and biologics (e.g., antibodies, peptide drugs, nucleic acid drugs). In certain embodiments, a chemotherapeutic agent does not include one or more of bevacizumab, ganetespib, temsirolimus, erlotinib, PTK787, BEZ235, XL765, pazopanib, cediranib, and axitinib.

As used herein, a “selected agent” is one or more of bevacizumab, ganetespib, temsirolimus, erlotinib, PTK787, BEZ235, XL765, pazopanib, cediranib, and axitinib. In certain embodiments, the selected agent is bevacizumab. In certain embodiments, the selected agent is ganetespib. In certain embodiments, the selected agent is temsirolimus. In certain embodiments, the selected agent is erlotinib. In certain embodiments, the selected agent is PTK787. In certain embodiments, the selected agent is BEZ235. In certain embodiments, the selected agent is XL765. In certain embodiments, the selected agent is pazopanib. In certain embodiments, the selected agent is cediranib. In certain embodiments, the selected agent is axitinib.

As used herein, “detecting”, “detection” and the like are understood that an assay performed for identification of a specific analyte in a sample, e.g., a hypoxia modulated polypeptide or a hypoxia modulated gene in a sample. The amount of analyte or activity detected in the sample can be none or below the level of detection of the assay or method.

The terms “modulate” or “modulation” refer to upregulation (i.e., activation or stimulation), downregulation (i.e., inhibition or suppression) of a level, or the two in combination or apart. A “modulator” is a compound or molecule that modulates, and may be, e.g., an agonist, antagonist, activator, stimulator, suppressor, or inhibitor.

The term “expression” is used herein to mean the process by which a polypeptide is produced from DNA. The process involves the transcription of the gene into mRNA and the translation of this mRNA into a polypeptide. Depending on the context in which used, “expression” may refer to the production of RNA, or protein, or both.

The terms “level of expression of a gene” or “gene expression level” refer to the level of mRNA, as well as pre-mRNA nascent transcript(s), transcript processing intermediates, mature mRNA(s) and degradation products, or the level of protein, encoded by the gene in the cell.

As used herein, “level of activity” is understood as the amount of protein activity, typically enzymatic activity, as determined by a quantitative, semi-quantitative, or qualitative assay. Activity is typically determined by monitoring the amount of product produced in an assay using a substrate that produces a readily detectable product, e.g., colored product, fluorescent product, radioactive product. For example, the isoforms of LDH in a sample can be resolved using gel electrophoresis. Lactate, nicotinamide adenine dinucleotide (NAD+), nitroblue tetrazolium (NBT), and phenazine methosulphate (PMS) can be added to assess LDH activity. LDH converts lactate to pyruvate and reduces NAD+ to NADH. The hydrogens from NADH are transferred by PMS to NBT reducing it to a purple formazan dye. The percentage of each LDH isoenzyme activity as well as the relative amount of each isoform to the other isoforms or total LDH can be determined, for example, by densitometry.

As used herein, “changed as compared to a control” sample or subject is understood as having a level of the analyte or diagnostic or therapeutic indicator (e.g., marker) to be detected at a level that is statistically different than a sample from a normal, untreated, or control sample Control samples include, for example, cells in culture, one or more laboratory test animals, or one or more human subjects. Methods to select and test control samples are within the ability of those in the art. An analyte can be a naturally occurring substance that is characteristically expressed or produced by the cell or organism (e.g., an antibody, a protein) or a substance produced by a reporter construct (e.g., β-galactosidase or luciferase). Depending on the method used for detection the amount and measurement of the change can vary. Changed as compared to a control reference sample can also include a change in one or more signs or symptoms associated with or diagnostic of disease, e.g., cancer. Determination of statistical significance is within the ability of those skilled in the art, e.g., the number of standard deviations from the mean that constitute a positive result.

As used herein, “binding” is understood as having at least a 10² or more, 10³ or more, preferably 10⁴ or more, preferably 10⁵ or more, preferably 10⁶ or more preference for binding to a specific binding partner as compared to a non-specific binding partner (e.g., binding an antigen to a sample known to contain the cognate antibody).

“Determining” as used herein is understood as performing an assay or using a diagnostic method to ascertain the state of someone or something, e.g., the presence, absence, level, or degree of a certain condition, biomarker, disease state, or physiological condition.

“Prescribing” as used herein is understood as indicating a specific agent or agents for administration to a subject.

As used herein, the terms “respond” or “response” are understood as having a positive response to treatment with a therapeutic agent, wherein a positive response is understood as having a decrease in at least one sign or symptom of a disease or condition (e.g., tumor shrinkage, decrease in tumor burden, inhibition or decrease of metastasis, improving quality of life (“QOL”), delay of time to progression (“TTP”), increase of overall survival (“OS”), etc.), or slowing or stopping of disease progression (e.g., halting tumor growth or metastasis, or slowing the rate of tumor growth or metastasis). A response can also include an improvement in quality of life, or an increase in survival time or progression free survival.

Ranges provided herein are understood to be shorthand for all of the values within the range. For example, a range of 1 to 50 is understood to include any number, combination of numbers, or sub-range from the group consisting 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, or 50.

Reference will now be made in detail to preferred embodiments of the invention. While the invention will be described in conjunction with the preferred embodiments, it will be understood that it is not intended to limit the invention to those preferred embodiments. To the contrary, it is intended to cover alternatives, modifications, and equivalents as may be included within the spirit and scope of the invention as defined by the appended claims.

### II. AGENTS FOR TREATMENT OF TUMORS WITH HIGH LEVELS OF HYPOXIA WITH SELECTED AGENTS

The invention provides methods of use of selected agents that are more effective in treating disease, e.g., cancer, when administered to a patient with a cancer or tumor exhibiting high levels of hypoxia. In one embodiment, the selected agent is selected from the group consisting of bevacizumab, ganetespib, temsirolimus, erlotinib, PTK787, BEZ235, XL765, pazopanib, cediranib, and axitinib. In an embodiment, the selected agent is ganetespib. In another embodiment, the selected agent is bevacizumab. In yet another embodiment, the selected agent is temsirolimus. In yet another embodiment, the selected agent is erlotinib. In yet another embodiment, the selected agent is pazopanib. In yet another embodiment, the selected agent is cediranib. In yet another embodiment, the selected agent is axitinib. In yet another embodiment, the selected agent is PTK787. In yet another embodiment, the selected agent is BEZ235. In yet another embodiment, the selected agent is XL765. In another embodiment, the selected agent selected from the group consisting of bevacizumab, ganetespib, temsirolimus, erlotinib, PTK787, BEZ235, XL765, pazopanib, cediranib, and axitinib is more effective in treating disease, e.g., cancer, when administered to a patient with a cancer or tumor exhibiting high levels of LDH.

A. Avastin®

Avastin®, also known as bevacizumab, R-435, and anti-VEGF, is a recombinant humanized monoclonal IgG1 antibody that binds to and inhibits the biologic activity of human vascular endothelial growth factor (VEGF). Bevacizumab contains human framework regions and the complementarity-determining regions of a murine antibody that binds to VEGF and is described in U.S. Pat. No. 6,054,297, the entire contents of which are expressly incorporated herein by reference. Bevacizumab is produced in a Chinese Hamster Ovary (CHO) mammalian cell expression system and has a molecular weight of approximately 149 kilodaltons. The light and heavy chains of bevacizumab have the following sequences:

>Fab-12, F(ab)-12, 12-IgG1,
rhuMAb-VEGFIIIVH-CH1 (VH(1-123) + CH1(124-215))
(SEQ ID NO: 1)
EVQLVESGGGLVQPGGSLRLSCAASGYTFTNYGMNWVRQAPGKGLEW
VGWINTYTGEPTYAADFKRRFTFSLDTSKSTAYLQMNSLRAEDTAVYYC
AKYPHYYGSSHWYFDVWGQGTLVTVSSASTKGPSVFPLAPSSKSTSGGT
AALGCLVKDYFPEPVTVSWNSGALTSGVHTFPAVLQSSGLYSLSSVVTV
PSSSLGTQTYICNVNHKPSNTKVDKKVEPKSCDKTHT
>Fab-12, F(ab)-12, 12-IgG1,
rhuMAb-VEGFIIIL-KAPPA (V-KAPPA(1-107) + C-
KAPPA(108-213))
(SEQ ID NO: 2)
DIQMTQSPSSLSASVGDRVTITCSASQDISNYLNWYQQKPGKAPKVLIYF
TSSLHSGVPSRFSGSGSGTDFTLTISSLQPEDFATYYCQQYSTVPWTFGQ
GTKVEIKRTVAAPSVFIFPPSDEQLKSGTASVVCLLNNFYPREAKVQWKV
DNALQSGNSQESVTEQDSKDSTYSLSSTLTLSKADYEKHKVYACEVTHQG
LSSPVTKSFNRGEC
>Fab-12, F(ab)-12, 12-IgG1,
rhuMAb-VEGFIIIVH-CH1 (VH(1-123) + CH1(124-215))
(SEQ ID NO: 3)
EVQLVESGGGLVQPGGSLRLSCAASGYTFTNYGMNWVRQAPGKGLEW
VGWINTYTGEPTYAADFKRRFTFSLDTSKSTAYLQMNSLRAEDTAVYYC
AKYPHYYGSSHWYFDVWGQGTLVTVSSASTKGPSVFPLAPSSKSTSGGT
AALGCLVKDYFPEPVTVSWNSGALTSGVHTFPAVLQSSGLYSLSSVVTV
PSSSLGTQTYICNVNHKPSNTKVDKKVEPKSCDKTHT
>Fab-12, F(ab)-12, 12-IgG1,
rhuMAb-VEGFIIIL-KAPPA (V-KAPPA(1- 107) + C-
KAPPA(108-213))
(SEQ ID NO: 4)
DIQMTQSPSSLSASVGDRVTITCSASQDISNYLNWYQQKPGKAPKVLIYF
TSSLHSGVPSRFSGSGSGTDFTLTISSLQPEDFATYYCQQYSTVPWTFGQ
GTKVEIKRTVAAPSVFIFPPSDEQLKSGTASVVCLLNNFYPREAKVQWKV
DNALQSGNSQESVTEQDSKDSTYSLSSTLTLSKADYEKHKVYACEVTHQG
LSSPVTKSFNRGEC

Bevacizumab is more effective in treating disease, e.g., cancer, when administered to a patient with a cancer or tumor exhibiting high levels of hypoxia. In another embodiment, bevacizumab is more effective in treating disease, e.g., cancer, when administered to a patient with a cancer or tumor exhibiting high levels of LDH.

B. Ganetespib

Ganetespib (also known as STA-9090) is a Heat Shock Protein 90 (Hsp90) inhibitor having the following structure.

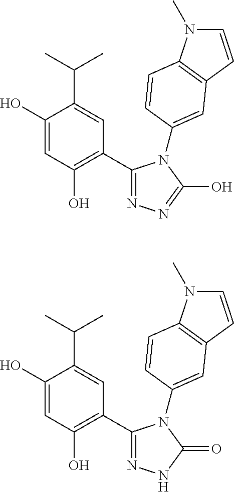 [SMILES: CC(C)c1cc(-c2nnc(O)n2-c2ccc3c(ccn3C)c2)c(O)cc1O ; CC(C)c1cc(-c2n[nH]c(=O)n2-c2ccc3c(ccn3C)c2)c(O)cc1O]

and the chemical name 3-2,4-dihydroxy-5-isopropyl-phenyl)-4-(1-methyl-indol-5-yl)-5-hydroxy-[1,2,4]-triazole (see, U.S. Pat. No. 7,825,148, incorporated herein by reference).

Hsp90 is a chaperone protein required for the proper folding and activation of other cellular proteins, particularly kinases, such as AKT, BCR-ABL, BRAF, KIT, MET, EGFR, FLT3, HER2, PDGFRA and VEGFR. These proteins have been shown to be critical to cancer cell growth, proliferation, and survival. Ganetespib has shown potent activity against a wide range of cancer types, including lung, prostate, colon, breast, gastric, pancreatic, gastrointestinal stromal tumors (GIST), melanoma, AML, chronic myeloid leukemia, Burkitt's lymphoma, diffuse large B-cell lymphoma and multiple myeloma in in vitro and in vivo models. Ganetespib has also shown potent activity against cancers resistant to imatinib, sunitinib, erlotinib and dasatinib.

Ganetespib is more effective in treating disease, e.g., cancer, when administered to a patient with a cancer or tumor exhibiting high levels of hypoxia. In another embodiment, ganetespib is more effective in treating disease, e.g., cancer, when administered to a patient with a cancer or tumor exhibiting high levels of LDH.

C. Torisel®

Torisel®, also known as CHI-779 or temsirolimus, is a compound having the structure:

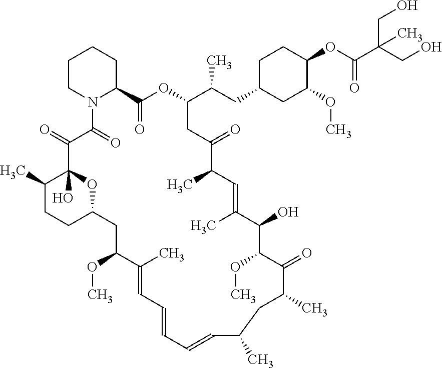 [SMILES: CO[C@H]1C[C@@H]2CC[C@@H](C)[C@](O)(O2)C(=O)C(=O)N2CCCC[C@H]2C(=O)O[C@H]([C@H](C)C[C@@H]2CC[C@@H](OC(=O)C(C)(CO)CO)[C@H](OC)C2)CC(=O)[C@H](C)/C=C(\C)[C@@H](O)[C@@H](OC)C(=O)[C@H](C)C[C@H](C)/C=C/C=C/C=C/1C]

Temsirolimus is an intravenous drug for the treatment of renal cell carcinoma (RACK), and is described in U.S. Pat. No. 5,362,718, the entire contents of which are expressly incorporated herein by reference. It is a derivative of birdlimes and is sold as Tories®. Temsirolimus is an inhibitor of mTOR (mammalian target of repaying). Temsirolimus binds to an intracellular protein (FKBP-12), and the protein-drug complex inhibits the activity of mTOR that controls cell division Inhibition of mTOR activity resulted in a G1 growth arrest in treated tumor cells. When mTOR was inhibited, its ability to phosphorylate p70S6K and S6 ribosomal protein, which are downstream of mTOR in the PI3 kinase/AKT pathway was blocked. In in vitro studies using renal cell carcinoma cell lines, temsirolimus inhibited the activity of mTOR and resulted in reduced levels of the hypoxia-inducible factors HIF-1 and HIF-2 alpha, and the vascular endothelial growth factor.

Temsirolimus is more effective in treating disease, e.g., cancer, when administered to a patient with a cancer or tumor exhibiting high levels of hypoxia. In another embodiment, temsirolimus is more effective in treating disease, e.g., cancer, when administered to a patient with a cancer or tumor exhibiting high levels of LDH.

D. Tarceva®

Tarceva®, also known as OSI-774 or erlotinib, has the chemical structure:

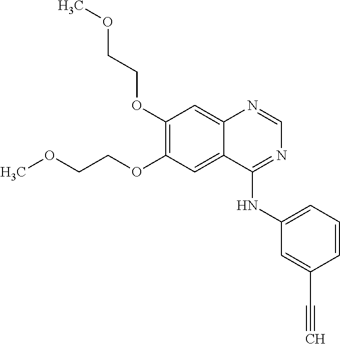 [SMILES: C#Cc1cccc(Nc2ncnc3cc(OCCOC)c(OCCOC)cc23)c1]

Erlotinib hydrochloride is used to treat non-small cell lung cancer, pancreatic cancer and several other types of cancer, and is described in U.S. Pat. Nos. 5,747,498; 6,900,221; 7,087,613 and RE41065, the entire contents of each of which are expressly incorporated herein by reference. Similar to gefitinib, erlotinib specifically targets the epidermal growth factor receptor (EGFR) tyrosine kinase. It binds in a reversible fashion to the adenosine triphosphate (ATP) binding site of the receptor. Erlotinib has recently been shown to be a potent inhibitor of JAK2V617F activity. JAK2V617F, a mutant of tyrosine kinase JAK2, is found in most patients with polycythemia vera (PV) and a substantial proportion of patients with idiopathic myelofibrosis or essential thrombocythemia.

Erlotinib is more effective in treating disease, e.g., cancer, when administered to a patient with a cancer or tumor exhibiting high levels of hypoxia. In another embodiment, erlotinib is more effective in treating disease, e.g., cancer, when administered to a patient with a cancer or tumor exhibiting high levels of LDH.

E. PTK787

PTK787, also known as vatalanib, PTK/ZK, ZK222584, CGP 78787D, or PTK7871, is a small molecule protein kinase inhibitor which inhibits angiogenesis. PTK787 inhibits all known VEGF receptors, platelet-derived growth factor β, and c-kit, and is orally active. The structure of PTK787 is shown below.

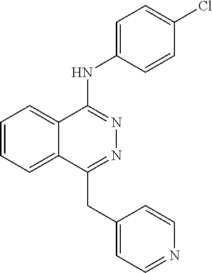 [SMILES: Clc1ccc(Nc2nnc(Cc3ccncc3)c3ccccc23)cc1]

### N-(4-chlorophenyl)-4-(pyridin-4-ylmethyl)phthalazin-1-amine

PTK787 can be used to treat metastatic colorectal cancer, both for patients with no prior treatment, and in subjects who had received first-line treatment with irinotecan and fluoropyrimidines. PTK787 can also be used to treat gastrointestinal stromal tumors, colorectal cancer, large cell lymphoma, meningioma, neuroendocrine tumors, solid tumors, acute myelogenous leukemia, agnogenic myeloid metaplasia, chronic myelogenous leukemia, Von Hippel-Lindau (VHL)-related hemangioblastoma, CNS hemangioblastoma, retinal hemangioblastoma, pancreatic cancer, prostate cancer, mesothelioma, glioblastoma, pancreatic adenocarcinoma, leukemia, brain tumors, central nervous system (CNS) tumors, glioblastoma multiforme, gastrointestinal carcinoid tumor, islet cell carcinoma, neuroendocrine tumors, extra-adrenal paraganglioma, gastrointestinal carcinoid tumor, head and neck cancer, islet cell tumor, lung cancer, melanoma, neuroendocrine carcinoma of the skin, pheochromocytoma, breast cancer, multiple myeloma, non-small cell lung cancer, gynecological cancers such as ovarian cancer, endometrial cancer, cervical cancer, fallopian tube cancer, and peritoneal cancer. PTK787 is described in PCT Publication No. WO98/35958 and U.S. Pat. Nos. 6,258,812; 6,514,974; 6,710,047 and 7,417,055, the entire contents of each of which are expressly incorporated herein by reference.

PTK787 is more effective in treating disease, e.g., cancer, when administered to a patient with a cancer or tumor exhibiting high levels of hypoxia. In another embodiment, PTK787 is more effective in treating disease, e.g., cancer, when administered to a patient with a cancer or tumor exhibiting high levels of LDH.

F. BEZ235

BEZ235, also known as NVP-BEZ235, is an orally bioavailable phosphatidylinositol 3-kinase (PI3K) inhibitor with potential antineoplastic activity. BEZ235 specifically inhibits PIK3 in the PI3K/AKT kinase (or protein kinase B) signaling pathway, which may trigger the translocation of cytosolic Bax to the mitochondrial outer membrane, increasing mitochondrial membrane permeability and leading to apoptotic cell death. Bax is a member of the proapoptotic Bc12 family of proteins. In addition to PI3K, NVP-BEZ235 also blocks mTOR kinase activity in biochemical assays [IC50=20.7 nM; K-LISA (kinase activity ELISA)] and the mTORC1 and mTORC2 kinase activity in immune-kinase assays. Accordingly, BEZ235 is able to significantly reduce the levels of phosphorylated RPS6 (ribosomal protein S6) in TSC1-deficient cells. See, e.g., Maira et al., Mol. Cancer. Ther., 7:1851-1863, 2008 and Maira et al., Biochem. Soc. Trans., 37:265-272, 2009.

The structure of BEZ235 is shown below.

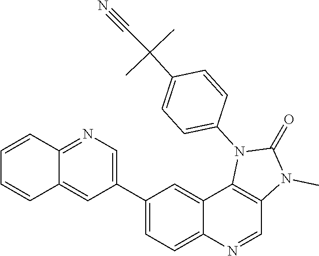 [SMILES: Cn1c(=O)n(-c2ccc(C(C)(C)C#N)cc2)c2c3cc(-c4cnc5ccccc5c4)ccc3ncc21]

BEZ235 is described in PCT Publication No. WO06/122806, U.S. Publication No. 2010/0056558 and U.S. Pat. No. 7,667,039, the entire contents of each of which are expressly incorporated herein by reference.

BEZ235 is more effective in treating disease, e.g., cancer, when administered to a patient with a cancer or tumor exhibiting high levels of hypoxia. In another embodiment, BEZ235 is more effective in treating disease, e.g., cancer, when administered to a patient with a cancer or tumor exhibiting high levels of LDH.

G. XL765

XL765, also known as SAR245409, is an orally available inhibitor of PI3K and the mammalian target of rapamycin (mTOR). PI3K plays an important role in cell proliferation and survival, and activation of the PI3K pathway is a frequent event in human tumors, promoting cell proliferation, survival, and resistance to chemotherapy and radiotherapy. mTOR is frequently activated in human tumors and plays a central role in tumor cell proliferation. The structure of XL765 is shown below.

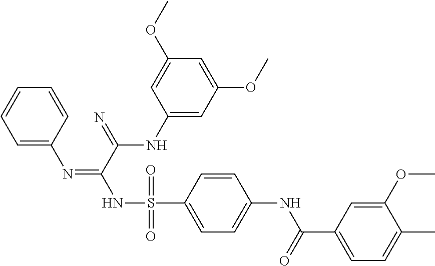 [SMILES: COc1cc(NC(=N)/C(=N\c2ccccc2)NS(=O)(=O)c2ccc(NC(=O)c3ccc(C)c(OC)c3)cc2)cc(OC)c1]

XL765 is more effective in treating disease, e.g., cancer, when administered to a patient with a cancer or tumor exhibiting high levels of hypoxia. In another embodiment, XL765 is more effective in treating disease, e.g., cancer, when administered to a patient with a cancer or tumor exhibiting high levels of LDH.

H. Pazopanib

Pazopanib, marketed under the name Votrient®, is a tyrosine kinase inhibitor (TKI). Pazopanib is presented as the hydrochloride salt, with the chemical name 5-[[4-[(2,3-dimethyl-2H-indazol-6-yl)methylamino]-2-pyrimidinyl]amino]-2-methylbenzenesulfonamide monohydrochloride. It has the molecular formula C₂₁N₇O₂S.HCl and a molecular weight of 473.99. Pazopanib-hydrochloride has the following chemical structure:

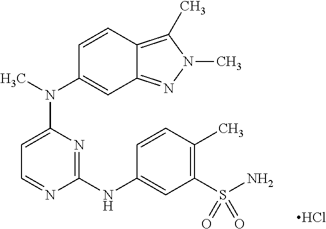 [SMILES: Cc1ccc(Nc2nccc(N(C)c3ccc4c(C)n(C)nc4c3)n2)cc1S(N)(=O)=O ; Cl]

Pazopanib is a multi-tyrosine kinase inhibitor of vascular endothelial growth factor receptor (VEGFR)-1, VEGFR-2, VEGFR-3, platelet-derived growth factor receptor (PDGFR)-α and -β, fibroblast growth factor receptor (FGFR)-1 and -3, cytokine receptor (Kit), interleukin-2 receptor inducible T-cell kinase (Itk), leukocyte-specific protein tyrosine kinase (Lck), and transmembrane glycoprotein receptor tyrosine kinase (c-Fms). In vitro, pazopanib inhibited ligand-induced autophosphorylation of VEGFR-2, Kit and PDGFR-β receptors. In vivo, pazopanib inhibited VEGF-induced VEGFR-2 phosphorylation in mouse lungs, angiogenesis in a mouse model, and the growth of some human tumor xenografts in mice.

Pazopanib is used for the treatment of renal cell carcinoma. Clinical trials for treatment of breast cancer including HER2 positive inflammatory breast cancer, neoplastic breast cancer, uterine cervical cancer, solid tumors, relapsed-refractory acute myelogenous leukemia, advanced kidney cancer, urothelial bladder cancer, non-small cell lung cancer, liver cancer, multiple myeloma, prostate cancer, malignant glioma, neuroendocrine tumors, and metastatic melanoma have been approved or performed.

Pazopanib is more effective in treating disease, e.g., cancer, when administered to a patient with a cancer or tumor exhibiting high levels of hypoxia. In another embodiment, pazopanib is more effective in treating disease, e.g., cancer, when administered to a patient with a cancer or tumor exhibiting high levels of LDH.

I. Cediranib

Cediranib, marketed under the name Recentin®, is a tyrosine kinase inhibitor (TKI). Cediranib is presented as the hydrochloride salt, with the chemical name 5-[[4-[(2,3-dimethyl-2H-indazol-6-yl]methylamino]-2-pyrimidinyl]amino]-2-methylbenzenesulfonamide monohydrochloride. It has the molecular formula C₂₁N₇O₂S.HCl and a molecular weight of 473.99. Cediranib-hydrochloride has the following chemical structure:

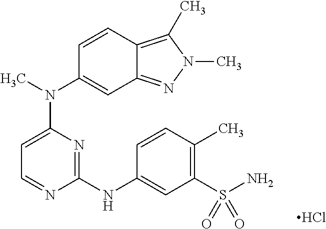 [SMILES: Cc1ccc(Nc2nccc(N(C)c3ccc4c(C)n(C)nc4c3)n2)cc1S(N)(=O)=O ; Cl]

Cediranib is a multi-tyrosine kinase inhibitor of vascular endothelial growth factor receptor (VEGFR)-1, VEGFR-2, VEGFR-3, platelet-derived growth factor receptor (PDGFR)-α and -β, fibroblast growth factor receptor (FGFR)-1 and -3, cytokine receptor (Kit), interleukin-2 receptor inducible T-cell kinase (Itk), leukocyte-specific protein tyrosine kinase (Lck), and transmembrane glycoprotein receptor tyrosine kinase (c-Fms). In vitro, cediranib inhibited ligand-induced autophosphorylation of VEGFR-2, Kit and PDGFR-β receptors. In vivo, cediranib inhibited VEGF-induced VEGFR-2 phosphorylation in mouse lungs, angiogenesis in a mouse model, and the growth of some human tumor xenografts in mice.

Cediranib is used for the treatment of renal cell carcinoma. Clinical trials for treatment of breast cancer including HER2 positive inflammatory breast cancer, neoplastic breast cancer, uterine cervical cancer, solid tumors, relapsed-refractory acute myelogenous leukemia, advanced kidney cancer, urothelial bladder cancer, non-small cell lung cancer, liver cancer, multiple myeloma, prostate cancer, malignant glioma, neuroendocrine tumors, and metastatic melanoma have been approved or performed.

Cediranib is more effective in treating disease, e.g., cancer, when administered to a patient with a cancer or tumor exhibiting high levels of hypoxia. In another embodiment, cediranib is more effective in treating disease, e.g., cancer, when administered to a patient with a cancer or tumor exhibiting high levels of LDH.

J. Axitinib

Axitinib (also known as AG013736), is a tyrosine kinase inhibitor (TKI) and has the chemical name N-Methyl-2-[[3-[(E)-2-pyridin-2-ylethenyl]-1H-indazol-6-yl]sulfanyl]benzamide, the molecular formula C₂₂H₁₈N₄OS and the molecular weight of 386.47 g/mol. Axitinib has the following chemical structure:

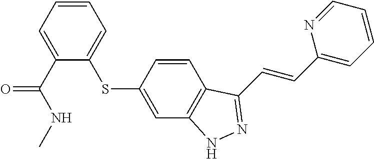 [SMILES: CNC(=O)c1ccccc1Sc1ccc2c(/C=C/c3ccccn3)n[nH]c2c1]

Axitinib inhibits multiple targets, including VEGFR-1, VEGFR-2, VEGFR-3, platelet derived growth factor receptor (PDGFR), and cKIT (CD117). It has been shown to significantly inhibit growth of breast cancer in xenograft models and has been successful in trials with renal cell carcinoma (RCC) and several other tumor types.

A Phase II clinical trial showed good response in combination chemotherapy with Gemcitabine for advanced pancreatic cancer. However, Pfizer reported on Jan. 30, 2009 that Phase III clinical trials of the drug when used in combination with Gemcitabine showed no evidence of improved survival rates over treatments using Gemcitabine alone for advanced pancreatic cancer and halted the trial.

In 2010 a Phase III trial for previously treated metastatic renal cell carcinoma (mRCC) showed significantly extended progression-free survival when compared to sorafenib.

Axitinib has been studied or approved for study in clinical trials for treatment of hepatocellular carcinoma, solid tumors, non-squamous non-small cell lung cancer in combination with pemetrexed and cisplatin; malignant mesothelioma, malignant pleural mesothelioma, renal cell cancer including metastatic renal cell cancer, in combination with paclitaxel and carboplatin in lung cancer including non-small-cell lung carcinoma and adenocarcinoma; metastatic, recurrent or primary unresectable adrenocortical cancer, adrenal cortex neoplasms, nasopharyngeal carcinoma, soft tissue sarcoma, in combination with FOLFOX or FOLFIRI for colorectal cancer, prostate cancer, melanoma, pancreatic cancer, gastric cancer, in conjunction with docetaxel for breast cancer, thyroid cancer, and acute myeloid leukemia (AML) or myelodysplastic syndrome.

K. Dosages and Modes of Administration

Techniques and dosages for administration vary depending on the type of compound (e.g., chemical compound, antibody, antisense, or nucleic acid vector) and are well known to those skilled in the art or are readily determined.

Therapeutic compounds of the present invention may be administered with a pharmaceutically acceptable diluent, carrier, or excipient, in unit dosage form. Administration may be parenteral, intravenous, subcutaneous, oral, or local by direct injection into the amniotic fluid. Administering an agent can be performed by a number of people working in concert. Administering an agent includes, for example, prescribing an agent to be administered to a subject and/or providing instructions, directly or through another, to take a specific agent, either by self-delivery, e.g., as by oral delivery, subcutaneous delivery, intravenous delivery through a central line, etc; or for delivery by a trained professional, e.g., intravenous delivery, intramuscular delivery, intratumoral delivery, etc.

The composition can be in the form of a pill, tablet, capsule, liquid, or sustained release tablet for oral administration; or a liquid for intravenous, subcutaneous, or parenteral administration; or a polymer or other sustained release vehicle for local administration.

Methods well known in the art for making formulations are found, for example, in “Remington: The Science and Practice of Pharmacy” (20th ed., ed. A. R. Gennaro, 2000, Lippincott Williams & Wilkins, Philadelphia, Pa.). Formulations for parenteral administration may, for example, contain excipients, sterile water, saline, polyalkylene glycols such as polyethylene glycol, oils of vegetable origin, or hydrogenated napthalenes. Biocompatible, biodegradable lactide polymer, lactide/glycolide copolymer, or polyoxyethylene-polyoxypropylene copolymers may be used to control the release of the compounds. Nanoparticulate formulations (e.g., biodegradable nanoparticles, solid lipid nanoparticles, liposomes) may be used to control the biodistribution of the compounds. Other potentially useful parenteral delivery systems include ethylene-vinyl acetate copolymer particles, osmotic pumps, implantable infusion systems, and liposomes. The concentration of the compound in the formulation varies depending upon a number of factors, including the dosage of the drug to be administered, and the route of administration.

The compound may be optionally administered as a pharmaceutically acceptable salts, such as non-toxic acid addition salts or metal complexes that are commonly used in the pharmaceutical industry. Examples of acid addition salts include organic acids such as acetic, lactic, pamoic, maleic, citric, malic, ascorbic, succinic, benzoic, palmitic, suberic, salicylic, tartaric, methanesulfonic, toluenesulfonic, or trifluoroacetic acids and the like; polymeric acids such as tannic acid, carboxymethyl cellulose, and the like; and inorganic acid such as hydrochloric acid, hydrobromic acid, sulfuric acid phosphoric acid, and the like. Metal complexes include zinc, iron, and the like.

Formulations for oral use include tablets containing the active ingredient(s) in a mixture with non-toxic pharmaceutically acceptable excipients. These excipients may be, for example, inert diluents or fillers (e.g., sucrose and sorbitol), lubricating agents, glidants, and anti-adhesives (e.g., magnesium stearate, zinc stearate, stearic acid, silicas, hydrogenated vegetable oils, or talc).

Formulations for oral use may also be provided as chewable tablets, or as hard gelatin capsules wherein the active ingredient is mixed with an inert solid diluent, or as soft gelatin capsules wherein the active ingredient is mixed with water or an oil medium.

The dosage and the timing of administering the compound depend on various clinical factors including the overall health of the subject and the severity of the symptoms of disease, e.g., cancer. In general, once a tumor is detected, administration of the agent is used to treat or prevent further progression of the tumor. Treatment can be performed for a period of time ranging from 1 to 100 days, more preferably 1 to 60 days, and most preferably 1 to 20 days, or until the remission of the tumor. It is understood that many chemotherapeutic agents are not administered daily, particularly agents with a long half-life. Therefore, an agent can be continually present without being administered daily. Dosages vary depending on each compound and the severity of the condition. Dosages can be titrated to achieve a steady-state blood serum concentration. Dosages can be interrupted or decreased in the presence of dose limiting toxicities.

### III. METHODS OF THE INVENTION

The instant invention provides methods of identifying a subject who will likely respond favorably to treatment with a selected agent by determining the level of hypoxia in a tumor, either by looking directly at markers within the tumor tissue or looking at markers in a peripheral sample from the subject, e.g., a bodily fluid such as blood, serum, plasma, lymph, urine, cerebrospinal fluid, or fecal matter, for the presence of one or more indicators of the level of hypoxia in the tumor.

The specific subject sample analyzed will depend, for example, on the site of the tumor. It is known that hypoxia drives angiogenesis in tumors, resulting in leaky blood vessels resulting in the presence of markers in circulation. Further, tumor growth and hypoxia are typically associated with necrosis and cell breakdown, resulting in cellular material in other bodily fluids or wastes. These readily accessible subject samples allow for the monitoring of the subject for the presence, or absence, of markers for hypoxia prior to and during the course of treatment.

Biopsies are routinely obtained for the purpose of cancer diagnosis, and solid tumors are frequently further resected prior to initiation of chemotherapy which also can be used for analysis to determine the level of hypoxia. Biopsy samples and resected tumor samples typically include at least some normal tissue adjacent to the tumor that can be used as a control.

In one embodiment of the invention, the modulated level of hypoxia is a high level of hypoxia. In one embodiment of the invention, the modulated level of hypoxia is a high level of LDH. In one embodiment, the level of hypoxia is determined by detecting the level of one or more hypoxia-modulated polypeptides or using one or more methods such as imaging methods. In one embodiment, a hypoxia-modulated polypeptide is at least one isoform or subunit of lactate dehydrogenase (LDH), at least one isoform or subunit of hypoxia inducible factor (HIF), at least one pro-angiogenic form of vascular endothelial growth factor (VEGF), phosphorylated VEGF receptor (pKDR), neurolipin 1 (NRP-1), pyruvate dehydrokinase (PDH-K), and ornithine decarboxylase (ODC). In one embodiment, the isoform or subunit of LDH is LDHH, LDH5, LDH4, LDH3, LDH2, LDH1 or LDHM, or any combination thereof. In another embodiment, the isoform or subunit of LDH is LDH5. In another embodiment, the level of hypoxia is determined by determining the ratio of two or more forms of LDH, e.g., the ratio of LDH5:LDH1. In another embodiment, the isoform of HIF is HIF-1α, HIF-1β, HIF-2α, and HIF-2β. In another embodiment, the pro-angiogenic isoform of VEGF is any one or a combination of VEGF-A splice variants. Antibodies against prodrugs that localize in hypoxic regions (e.g., EF5, pimonidazole, etc.) can also be used to detect hypoxia. Tumor size can also be correlated with a level of hypoxia. A level of hypoxia can also be determined by PET scan. Functional imaging measuring blood flow in the tumor can be used as an indicator of hypoxia in the tissue. Direct measurement of hypoxia can be preformed by inserting a sensor into the tumor.

Methods to detect the protein or activity levels of markers of hypoxia, or hypoxia modulated polypeptides, are well known in the art. Antibodies against and kits for detection of hypoxia modulated polypeptides can be purchased from a number of commercial sources. Alternatively, using routine methods known in the art (e.g., immunization of animals, phage display, etc.) antibodies against one or more hypoxia modulated polypeptides or subunits or isoforms thereof can be made and characterized. Antibodies can be used for the detection of levels of hypoxia using ELISA, RIA, or other immunoassay methods, preferably automated methods, for the quantitative detection of proteins in samples of bodily fluids or homogenized solid samples. Alternatively, immunohistochemical methods can be used on tumor samples and tissue sections. Qualitative scoring methods and scanning methods to detect staining are known in the art. When qualitative scoring methods are used, it is preferred that two independent, blinded technicians, pathologists, or other skilled individuals analyze each sample with specific methods for resolving any significant disagreement in scoring, e.g., a third individual reviews the tissue sample. Many markers of hypoxia, including LDH, are enzymes. Enzymatic activity can be assayed in total, or for individual isoforms, for example, using in gel assays.

Alternatively, nucleic acid based methods of detection of levels of hypoxia are also well known in the art. Methods of designing primers and probes for quantitative reverse transcription real time (rt) PCR are known in the art. Methods for performing northern blots to detect RNA levels are known in the art. Nucleic acid detection methods can also include fluorescence in situ hybridization (FISH) and in situ PCR. Qualitative scoring methods and scanning methods to detect staining are known in the art.

In another aspect, the present invention provides methods for the preselection of a subject for therapeutic treatment with an anti-cancer agent, wherein the subject has previously been found to have a high level of hypoxia. The invention also provides methods for the preselection of a subject for therapeutic treatment with an the agent by evaluating the results of an assessment of a sample from the subject for a high level of hypoxia.

Such determinations can be made based on a chart review of the level of hypoxia of the tumor of the subject. Inclusion criteria can include information being available regarding the cancer type, the specific treatment regimen with the agent, and the outcome to death or for a meaningful follow-up period which varies depending on the cancer type, e.g., metastatic or refractile cancers with poor prognoses requiring follow-up of weeks to months whereas cancers with less poor prognoses preferably having months to years of follow-up with subjects. In addition to information related to survival, information related to quality of life, side effects, and other relevant information can be considered when available. Exclusion criteria can include the presence of other diseases or conditions that could result in alteration of levels of hypoxia modulated peptides, e.g., ischemic heart or vascular disease, poor circulation, diabetes, macular degeneration, recent stroke, or other ischemic events or conditions. Other exclusion criteria can be selected based on the available samples and patient population, e.g., prior treatment with specific agents.

The subjects can be sorted into groups based on various criteria. Subjects who were treated with an agent for whom no levels of hypoxic markers were determined can be used as an unstratified control group to understand the efficacy of the agent on a treatment population not selected based on the level of hypoxia in the subject. Alternatively, the population analyzed in the study can be compared to historical control samples in which an unstratified population was analyzed for response to the agent.

Subjects for whom hypoxic levels were obtained can be divided into two or more groups having high and low level of hypoxia, optionally with a group of subjects with moderate levels of hypoxia, depending on the distribution of subjects. It is understood that subjects and samples can also be divided into other groups, e.g., survival time, treatment regimen with the agent, cancer type, previous failed treatments, etc. for analysis. Preferably, the same marker(s) of hypoxia is measured in each of the subjects, e.g., at least one isoform or subunit of lactate dehydrogenase (LDH) or hypoxia inducible factor (HIF); at least one pro-angiogenic form of vascular endothelial growth factor (VEGF), phosphorylated VEGF receptor (pKDR) 1, 2, or 3; GLUT-1, GLUT-2, neurolipin 1 (NRP-1), pyruvate dehydrokinase (PDH-K), and ornithine decarboxylase (ODC). Tumor size can also be a marker correlated with a level of hypoxia. A marker of a level of hypoxia can also be determined by PET scan. A level of hypoxia can also be determined by PET scan. Further, it is preferred that the same type of subject sample, e.g., blood, serum, lymph, tumor tissue, etc., is tested for the presence of the marker for the level of hypoxia. It is understood that the level of hypoxia can be measured directly in the tumor sample, using quantitative, semi-quantitative, or qualitative immunohistochemical methods, immunological assays (e.g., ELISA assay); reverse transcription PCR assays, particularly quantitative PCR methods, e.g., real time PCR; northern blot assays, enzyme activity assays (e.g., for lactate dehydrogenase activity, for kinase activity); and in situ hybridization assay (e.g., fluorescence in situ hybridization (FISH) assay). Antibodies against prodrugs that localize in hypoxic regions (e.g., EF5, pimonidazole, etc.) can also be used to detect hypoxia. Functional imaging measuring blood flow in the tumor can be used as an indicator of hypoxia in the tissue. Direct measurement of hypoxia can be preformed by inserting a sensor into the tumor. Antibodies against prodrugs that localize in hypoxic regions (e.g., EF5, pimonidazole, etc.) can also be used as markers to detect hypoxia. Functional imaging measuring blood flow in the tumor can be used as a marker of hypoxia in the tissue. Direct measurement of hypoxia can be preformed to provide a marker for hypoxia by inserting a sensor into the tumor. Again, it is preferred that the same method of determining the level of the marker of hypoxia is used for all samples, particularly when qualitative assessment methods are used.

Outcomes of subjects based on the level of hypoxia can be analyzed to determine if the outcome between the two groups is different. Outcomes can further be compared to a non-stratified group treated with the agent. Methods for statistical analysis and determination of statistical significance are within the ability of those of skill in the art. The analysis demonstrates that subjects with a high level of hypoxia have a better response, e.g., one or more of longer time to failure, longer survival time, better quality of life, decreased tumor size, better tolerance of the agent, etc., as compared to subjects with a low level of hypoxia.

In another aspect, the present invention provides methods for the preselection of a subject for therapeutic treatment with a selected agent, wherein the subject has previously been found to have a high level of hypoxia. The invention also provides methods for the preselection of a subject for therapeutic treatment with a selected agent by evaluating the results of an assessment of a sample from the subject for a modulated level of hypoxia wherein the subject is found to have a high level of hypoxia. Such determinations can be made based on the level of hypoxia observed in historical samples. An analysis using samples collected from subjects during treatment can be performed to determine the efficacy of a selected agent for the treatment of cancer based on the level of hypoxia of the tumor based on markers assessed during the treatment of the subjects. Inclusion criteria are information being available regarding the cancer type, the specific treatment regimen with the selected agent, and the outcome to death or for a meaningful follow-up period which varies depending on the cancer type, e.g., metastatic or refractile cancers with poor prognoses requiring follow-up of weeks to months whereas cancers with less poor prognoses preferably having months to years of follow-up with subjects. In addition to information related to survival, information related to quality of life, side effects, and other relevant information is considered when available. Exclusion criteria can include the presence of other diseases or conditions that could result in alteration of levels of hypoxia modulated peptides, e.g., ischemic heart or vascular disease, poor circulation, diabetes, macular degeneration, recent stroke, or other ischemic events or conditions. Other exclusion criteria can be selected based on the available samples and patient population, e.g., prior treatment with specific agents.

The samples can be analyzed for the level of hypoxia. Preferably, all of the samples are the same type or types, e.g., blood, plasma, lymph, tumor tissue. Depending on the availability of subject samples, the analysis can be performed using two (or more) subject sample types, e.g., serum and tumor tissue. Various portions of the tumor tissue can also be analyzed when sufficient material is available, e.g., adjacent to the necrotic core, in the center of the tumor, adjacent to or including tumor vasculature, adjacent to normal tissue, etc. One or more markers of hypoxia can be measured in each of the subjects, e.g., at least one isoform or subunit of lactate dehydrogenase (LDH) or hypoxia inducible factor (HIF); at least one pro-angiogenic form of vascular endothelial growth factor (VEGF), phosphorylated VEGF receptor (pKDR) 1, 2, or 3, GLUT-1, GLUT-2, neurolipin 1 (NRP-1), pyruvate dehydrokinase (PDH-K), and ornithine decarboxylase (ODC). Enzymatic assays of markers can be performed. Tumor size can also be a marker correlated with a level of hypoxia. A marker of a level of hypoxia can also be determined by PET scan. Antibodies against prodrugs that localize in hypoxic regions (e.g., EF5, pimonidazole, etc.) can also be used as markers to detect hypoxia. Functional imaging measuring blood flow in the tumor can be used as a marker of hypoxia in the tissue. Direct measurement of hypoxia can be preformed to provide a marker for hypoxia by inserting a sensor into the tumor. Further, it is preferred that the same type of subject sample, e.g., blood, serum, lymph, tumor tissue, etc., is tested for the presence of the marker for the level of hypoxia. It is understood that the level of hypoxia could have been measured directly in the tumor sample, using quantitative, semi-quantitative, or qualitative immunohistochemical methods, immunological assays (e.g., ELISA assay); reverse transcription PCR assays, particularly quantitative PCR methods, e.g., real time PCR; northern blot assays, enzyme activity assays (e.g., for lactate dehydrogenase activity, for kinase activity); and in situ hybridization assay (e.g., fluorescence in situ hybridization (FISH) assay). Again, it is preferred that the same method of determining the level of the marker of hypoxia is used for all samples, particularly when qualitative assessment methods are used.

In another aspect, the present invention provides methods for treating a cancer with selected agents selected from the group consisting of bevacizumab, ganetespib, temsirolimus, erlotinib, PTK787, BEZ235, XL765, pazopanib, cediranib, and axitinib in a subject having a high level of hypoxia. The methods include not administering to the subject having a cancer or susceptible to a cancer who further has a low level of hypoxia, a selected agent selected from the group consisting of bevacizumab, ganetespib, temsirolimus, erlotinib, PTK787, BEZ235, XL765, pazopanib, cediranib, and axitinib, thereby treating the cancer. Other methods include administering to the subject having a cancer or susceptible to a cancer a selected agent selected from the group consisting of bevacizumab, ganetespib, temsirolimus, erlotinib, PTK787, BEZ235, XL765, pazopanib, cediranib, and axitinib, and at least one chemotherapeutic agent, thereby treating the cancer. In certain embodiments, the subject has previously been treated with a chemotherapeutic agent.

Other methods include methods of treating a subject who has cancer by prescribing to the subject an effective amount of a selected agent selected from the group consisting of bevacizumab, ganetespib, temsirolimus, erlotinib, PTK787, BEZ235, XL765, pazopanib, cediranib, and axitinib, wherein the subject has previously been found to have a high level of hypoxia. As used herein, the term “prescribing” is understood as indicating a specific agent or agents for administration to a subject. Furthermore, the present invention also includes methods of increasing the likelihood of effectively treating a subject having cancer by administering a therapeutically effective amount of a composition comprising a selected agent selected from the group consisting of bevacizumab, ganetespib, temsirolimus, erlotinib, PTK787, BEZ235, XL765, pazopanib, cediranib, and axitinib, to the subject, wherein the subject has previously been found to have a modulated level of hypoxia.

Cancers that may be treated or prevented using the methods of the invention include, for example, acoustic neuroma, acute leukemia, acute lymphocytic leukemia, acute myelocytic leukemia (monocytic, myeloblastic, adenocarcinoma, angiosarcoma, astrocytoma, myelomonocytic and promyelocytic), acute T-cell leukemia, basal cell carcinoma, bile duct carcinoma, bladder cancer, brain cancer, breast cancer, bronchogenic carcinoma, cervical cancer, chondrosarcoma, chordoma, choriocarcinoma, chronic leukemia, chronic lymphocytic leukemia, chronic myelocytic (granulocytic) leukemia, chronic myleogeneous leukemia, colon cancer, colorectal cancer, craniopharyngioma, cystadenocarcinoma, diffuse large B-cell lymphoma, dysproliferative changes (dysplasias and metaplasias), embryonal carcinoma, endometrial cancer, endotheliosarcoma, ependymoma, epithelial carcinoma, erythroleukemia, esophageal cancer, estrogen-receptor positive breast cancer, essential thrombocythemia, Ewing's tumor, fibrosarcoma, follicular lymphoma, germ cell testicular cancer, glioma, heavy chain disease, hemangioblastoma, hepatoma, hepatocellular cancer, hormone insensitive prostate cancer, leiomyosarcoma, liposarcoma, lung cancer, lymphagioendotheliosarcoma, lymphangiosarcoma, lymphoblastic leukemia, lymphoma (Hodgkin's and non-Hodgkin's), malignancies and hyperproliferative disorders of the bladder, breast, colon, lung, ovaries, pancreas, prostate, skin and uterus, lymphoid malignancies of T-cell or B-cell origin, leukemia, lymphoma, medullary carcinoma, medulloblastoma, melanoma, meningioma, mesothelioma, multiple myeloma, myelogenous leukemia, myeloma, myxosarcoma, neuroblastoma, non-small cell lung cancer, oligodendroglioma, oral cancer, osteogenic sarcoma, ovarian cancer, pancreatic cancer, papillary adenocarcinomas, papillary carcinoma, pinealoma, polycythemia vera, prostate cancer, rectal cancer, renal cell carcinoma, retinoblastoma, rhabdomyosarcoma, sarcoma, sebaceous gland carcinoma, seminoma, skin cancer, small cell lung carcinoma, solid tumors (carcinomas and sarcomas), small cell lung cancer, stomach cancer, squamous cell carcinoma, synovioma, sweat gland carcinoma, thyroid cancer, Waldenstrom's macroglobulinemia, testicular tumors, uterine cancer and Wilms' tumor. Other cancers include primary cancer, metastatic cancer, oropharyngeal cancer, hypopharyngeal cancer, liver cancer, gallbladder cancer, small intestine cancer, urinary tract cancer, kidney cancer, urothelium cancer, female genital tract cancer, uterine cancer, gestational trophoblastic disease, male genital tract cancer, seminal vesicle cancer, testicular cancer, germ cell tumors, endocrine gland tumors, thyroid cancer, adrenal cancer, and pituitary gland cancer, hemangiomas, sarcomas arising from bone and soft tissues; Kaposi's sarcoma, nerve cancer, ocular cancer, and meningial cancer, glioblastomas, neuromas, Schwannomas, solid tumors arising from hematopoietic malignancies such as leukemias, metastatic melanoma, recurrent or persistent ovarian epithelial cancer, fallopian tube cancer, primary peritoneal cancer, gastrointestinal stromal tumors, colorectal cancer, gastric cancer, melanoma, glioblastoma multiforme, non-squamous non-small-cell lung cancer, malignant glioma, epithelial ovarian cancer, primary peritoneal serous cancer, metastatic liver cancer, neuroendocrine carcinoma, refractory malignancy, triple negative breast cancer, HER2 amplified breast cancer, squamous cell carcinoma of the head and neck (SCCHN), nasopharageal cancer, oral cancer, biliary tract, hepatocellular carcinoma, non-medullary thyroid carcinoma, recurrent glioblastoma multiforme, neurofibromatosis type 1, CNS cancer, liposarcoma; leiomyosarcoma; salivary gland cancer, mucosal melanoma; acral/lentiginous melanoma, paraganglioma, and pheochromocytoma.

It is understood that diagnosis and treatment of a complex disease such as cancer is not performed by a single individual, test, agent, or intervention. For example, a subject may meet with a primary care physician to express a concern and be referred to an oncologist who will request tests that are designed, carried out, and analyzed by any of a number of individuals, but not limited to, radiologists, radiology technicians, physicists, phlebotomists, pathologists, laboratory technicians, and radiation, clinical, and surgical oncologists. Selection, dosing, and administration of agents to a subject diagnosed with cancer will be performed by any of a number of individuals including, but not limited to, radiologists, radiology technicians, physicists, pathologists, infusion nurses, pharmacists, and radiation, clinical, and surgical oncologists. Therefore, it is understood that within the terms of the invention, identifying a subject as having a specific level of hypoxia can include any of a number of acts including, but not limited to, performing a test and observing a result that is indicative of a subject having a specific level of hypoxia; reviewing a test result of a subject and identifying the subject as having a specific level of hypoxia; reviewing documentation on a subject stating that the subject has a specific level of hypoxia and identifying the subject as the one discussed in the documentation by confirming the identity of the subject, e.g., by an identification card, hospital bracelet, asking the subject for his/her name and/or other personal information to confirm the subjects identity.

Similarly, administering an agent can be performed by a number of people working in concert. Administering an agent includes, for example, prescribing an agent to be administered to a subject and/or providing instructions, directly or through another, to take a specific agent, either by self-delivery, e.g., as by oral delivery, subcutaneous delivery, intravenous delivery through a central line, etc; or for delivery by a trained professional, e.g., intravenous delivery, intramuscular delivery, intratumoral delivery, etc.

As discussed extensively, above, the terms “administer”, “administering” or “administration” include any method of delivery of a pharmaceutical composition or agent into a subject's system or to a particular region in or on a subject. In certain embodiments of the invention, an agent is administered intravenously, intramuscularly, subcutaneously, intradermally, intranasally, orally, transcutaneously, or mucosally. In a preferred embodiment, an agent is administered intravenously. Administering an agent can be performed by a number of people working in concert. Administering an agent includes, for example, prescribing an agent to be administered to a subject and/or providing instructions, directly or through another, to take a specific agent, either by self-delivery, e.g., as by oral delivery, subcutaneous delivery, intravenous delivery through a central line, etc.; or for delivery by a trained professional, e.g., intravenous delivery, intramuscular delivery, intratumoral delivery, etc.

### IV. KITS OF THE INVENTION

The invention also provides for kits to practice the methods of the invention. For example, a kit can include a selected agent selected from the group consisting of bevacizumab, ganetespib, temsirolimus, erlotinib, PTK787, BEZ235, XL765, pazopanib, cediranib, and axitinib, and an instruction for administration of the selected agent to a subject having cancer with a high level of hypoxia. In another embodiment, the subject has cancer with a high level of lactate dehydrogenase (LDH). In one embodiment, the instruction provides that the selected agent selected from the group consisting of bevacizumab, ganetespib, temsirolimus, erlotinib, PTK787, BEZ235, XL765, pazopanib, cediranib, and axitinib, is a second line therapy. In another example, the kits of the invention may contain reagents for determining the level of LDH in a sample from a subject.

### EXAMPLES

### Example 1

### Selection of Subjects for Treatment with a Selected Agent Based on a Level of Hypoxia

A subject is diagnosed with a cancer based on a series of clinically accepted diagnostic criteria including imaging, immunohistochemistry, hematological analyses, and physical examination. The immunohistochemical analysis includes staining for the presence of one or more hypoxic markers in the biopsy sample. Further, or alternatively, a serum sample is tested for the presence of one or more hypoxic markers.

A subject is identified as having a high level of a hypoxic marker in serum and/or in the tumor. The subject is selected for treatment with the agent known to be effective in treating cancer in a subject having a high level of hypoxic marker. The subject is treated with the selected agent selected from the group consisting of bevacizumab, ganetespib, temsirolimus, erlotinib, PTK787, BEZ235, XL765, pazopanib, cediranib, and axitinib, and monitored for therapeutic response as well as the presence of side effects. Therapy is continued as long as it is sufficiently tolerated and a benefit to the subject is observed as determined by the subject, the treating physician, the caregiver, and/or other qualified individual.

### Example 2

### Selection of Subjects not to be Treated with a Selected Agent Based on a Level of Hypoxia

A subject is diagnosed with cancer based on a series of clinically accepted diagnostic criteria including imaging, immunohistochemistry, hematological analyses, and physical examination. The immunohistochemical analysis includes staining for the presence of one or more hypoxic markers in the biopsy sample. Further, or alternatively, a serum sample is tested for the presence of one or more hypoxic markers.

A subject is identified as having a low level of a hypoxic marker in serum and/or in the tumor. A treatment regimen not including a selected agent selected from the group consisting of bevacizumab, ganetespib, temsirolimus, erlotinib, PTK787, BEZ235, XL765, pazopanib, cediranib, and axitinib, known to be effective in treating cancer in a subject having a high level of hypoxic marker is selected for the subject.

### Example 3

### Characterization of Treatment Outcomes Based on Chart Review

A chart review analysis is performed to determine the efficacy of a selected agent selected from the group consisting of bevacizumab, ganetespib, temsirolimus, erlotinib, PTK787, BEZ235, XL765, pazopanib, cediranib, and axitinib, for the treatment of a cancer based on the level of hypoxia of the tumor based on markers assessed during the treatment of the subjects. Inclusion criteria are information being available regarding the cancer type, the specific treatment regimen with the selected agent, and the outcome over a meaningful follow-up period which varies depending on the cancer type, e.g., metastatic or refractile cancers with poor prognoses requiring follow-up of weeks to months (e.g., until death, until tumor progression, until administration of new therapeutic intervention) whereas cancers with less poor prognoses preferably having months to years of follow-up with subjects (e.g., until tumor progression, until administration of new therapeutic intervention, to an arbitrary end point). In addition to information related to survival, information related to quality of life, side effects, and other relevant information is considered when available. Exclusion criteria can include the presence of other diseases or conditions that could result in alteration of levels of hypoxia modulated peptides, e.g., ischemic heart or vascular disease, poor circulation, diabetes, macular degeneration, recent stroke, recent surgery, or other ischemic events or conditions. Other exclusion criteria can be selected based on the available samples and patient population, e.g., prior treatment with specific agents.

The subjects can be sorted into groups based on various criteria. Subjects who were treated with a selected agent selected from the group consisting of bevacizumab, ganetespib, temsirolimus, erlotinib, PTK787, BEZ235, XL765, pazopanib, cediranib, and axitinib, for whom no levels of hypoxic markers were determined can be used as an unstratified control group to understand the efficacy of the selected agent on a treatment population not selected based on the level of hypoxia in the subject/tumor. Alternatively, the population analyzed in the study for which hypoxia levels (e.g., LDH marker levels) can be compared to historical control samples in which an unstratified population was analyzed for response to the agent.

Subjects for whom hypoxic levels are available in chart records are divided into two or more groups having high and low level of hypoxia, optionally with a group of subjects with moderate levels of hypoxia, depending on the distribution of subjects. It is understood that subjects and samples can also be divided into other groups, e.g., survival time, treatment regimen with the selected agent, cancer type, previous failed treatments, etc. for analysis. Preferably, the same marker(s) of hypoxia is measured in each of the subjects, e.g., at least one isoform or subunit of lactate dehydrogenase (LDH) or hypoxia inducible factor (HIF); at least one pro-angiogenic form of vascular endothelial growth factor (VEGF), phosphorylated VEGF receptor (pKDR) 1, 2, or 3; neurolipin 1 (NRP-1), pyruvate dehydrokinase (PDH-K), and ornithine decarboxylase (ODC). Antibodies against prodrugs that localize in hypoxic regions (e.g., EF5, pimonidazole, etc.) can also be markers hypoxia. Functional imaging measuring blood flow in the tumor can be used as a marker of hypoxia in the tissue. Direct measurement of hypoxia can be a marker and can be preformed by inserting a sensor into the tumor. Tumor size can also be a marker correlated with hypoxia. Further, it is preferred that the same type of subject sample, e.g., blood, serum, lymph, tumor tissue, etc., is tested for the presence of the marker for the level of hypoxia. It is understood that the level of hypoxia can be measured directly in the tumor sample, using quantitative, semi-quantitative, or qualitative immunohistochemical methods, immunological assays (e.g., ELISA assay); reverse transcription PCR assays, particularly quantitative PCR methods, e.g., real time PCR; northern blot assays, enzyme activity assays (e.g., for lactate dehydrogenase activity, for kinase activity); and in situ hybridization assay (e.g., fluorescence in situ hybridization (FISH) assay). Antibodies against prodrugs that localize in hypoxic regions (e.g., EF5, pimonidazole, etc.) can also be used to detect hypoxia. PET scans can be used to detect hypoxia. Functional imaging measuring blood flow in the tumor can be used as an indicator of hypoxia in the tissue. Direct measurement of hypoxia can be preformed by inserting a sensor into the tumor. Tumor size can also be a marker for hypoxia. Again, it is preferred that the same method of determining the level of the marker of hypoxia is used for all samples, particularly when qualitative assessment methods are used.

Outcomes of subjects based on the level of hypoxia are analyzed to determine if the outcome between the two groups is different. Outcomes can further be compared to a non-stratified group treated with the selected agent selected from the group consisting of bevacizumab, ganetespib, temsirolimus, erlotinib, PTK787, BEZ235, XL765, pazopanib, cediranib, and axitinib. Methods for statistical analysis and determination of statistical significance are within the ability of those of skill in the art. For the selected agents, the analysis demonstrates that subjects with a high level of hypoxia have a better response, e.g., one or more of longer time to failure, longer survival time, better quality of life, decreased tumor size, better tolerance of the selected agent, etc., as compared to subjects with a low level of hypoxia, and that such agents should be preferentially used in subjects having high levels of markers of hypoxia.

### Example 4

### Characterization of Treatment Outcomes Based on Historical Samples

An analysis using samples collected from subjects during treatment is performed to determine the efficacy of a selected agent selected from the group consisting of bevacizumab, ganetespib, temsirolimus, erlotinib, PTK787, BEZ235, XL765, pazopanib, cediranib, and axitinib, for the treatment of cancer based on the level of hypoxia of the tumor based on markers assessed prior to and/or during the treatment of the subjects. Inclusion criteria are information being available regarding the cancer type, the specific treatment regimen with the selected agent, and the outcome for a meaningful follow-up period which varies depending on the cancer type, e.g., metastatic or refractile cancers with poor prognoses requiring follow-up of weeks to months (e.g., until death, until tumor progression, until administration of new therapeutic intervention) whereas cancers with less poor prognoses preferably having months to years of follow-up (e.g., until tumor progression, until administration of new therapeutic intervention, to an arbitrary end point) with subjects. In addition to information related to survival, information related to quality of life, side effects, and other relevant information is considered when available. Exclusion criteria include the presence of other diseases or conditions that could result in alteration of levels of hypoxia modulated peptides, e.g., ischemic heart or vascular disease, poor circulation, diabetes, macular degeneration, recent stroke, or other ischemic events or conditions. Other exclusion criteria can be selected based on the available samples and patient population, e.g., prior treatment with specific agents.

The samples are analyzed for the level of hypoxia. Preferably, all of the samples are the same type or types, e.g., blood, plasma, lymph, urine, tumor tissue. Depending on the availability of subject samples, the analysis can be performed using two (or more) subject sample types, e.g., serum and tumor tissue. Various portions of the tumor tissue can also be analyzed when sufficient material is available, e.g., adjacent to the necrotic core, in the center of the tumor, adjacent to or including tumor vasculature, adjacent to normal tissue, etc. One or more markers of hypoxia are measured in each of the subjects, e.g., at least one isoform or subunit of lactate dehydrogenase (LDH) or hypoxia inducible factor (HIF); at least one pro-angiogenic form of vascular endothelial growth factor (VEGF), phosphorylated VEGF receptor (pKDR) 1, 2, or 3, neurolipin 1 (NRP-1), pyruvate dehydrokinase (PDH-K), and ornithine decarboxylase (ODC), tumor size. Antibodies against prodrugs that localize in hypoxic regions (e.g., EF5, pimonidazole, etc.) can also be markers hypoxia. Functional imaging measuring blood flow in the tumor can be used as a marker of hypoxia in the tissue. Direct measurement of hypoxia can be a marker and can be preformed by inserting a sensor into the tumor. Tumor size can also be a marker correlated with hypoxia. Further, it is preferred that the same type of subject sample, e.g., blood, serum, lymph, urine, tumor tissue, etc., is tested for the presence of the marker for the level of hypoxia. It is understood that the level of hypoxia can be measured directly in the tumor sample, using quantitative, semi-quantitative, or qualitative immunohistochemical methods, immunological assays (e.g., ELISA assay); reverse transcription PCR assays, particularly quantitative PCR methods, e.g., real time PCR; northern blot assays, enzyme activity assays (e.g., for lactate dehydrogenase activity, for kinase activity); and in situ hybridization assay (e.g., fluorescence in situ hybridization (FISH) assay). Antibodies against prodrugs that localize in hypoxic regions (e.g., EF5, pimonidazole, etc.) can also be used to detect hypoxia. PET scans can be used to detect hypoxia. Functional imaging measuring blood flow in the tumor can be used as an indicator of hypoxia in the tissue. Direct measurement of hypoxia can be preformed by inserting a sensor into the tumor. Tumor size can also be a marker for hypoxia. Again, it is preferred that the same method of determining the level of the marker of hypoxia was determined using the same method in all samples, particularly when qualitative assessment methods are used.

Subjects are divided into two or more groups having high and low level of hypoxia, optionally with a group of subjects with moderate levels of hypoxia, depending on the distribution of subjects. It is understood that subjects and samples can also be divided into other groups, e.g., survival time, treatment regimen with the selected agent selected from the group consisting of bevacizumab, ganetespib, temsirolimus, erlotinib, PTK787, BEZ235, XL765, pazopanib, cediranib, and axitinib, cancer type, previous failed treatments, etc. for analysis.

Outcomes of subjects based on the level of hypoxia are analyzed to determine if the outcome between the two groups is different. Outcomes can further be compared to a non-stratified group treated with the selected agent selected from the group consisting of bevacizumab, ganetespib, temsirolimus, erlotinib, PTK787, BEZ235, XL765, pazopanib, cediranib, and axitinib, e.g., a historical group provided by another study. Methods for statistical analysis and determination of statistical significance are within the ability of those of skill in the art. For the selected agents, the analysis demonstrates that subjects with a high level of hypoxia have a better response, e.g., one or more of longer time to failure, longer survival time, better quality of life, decreased tumor size, better tolerance of the selected agent, delayed time to progression, etc., as compared to subjects with a low level of hypoxia, and that such agents should be preferentially used in subjects having high levels of markers of hypoxia.

### Example 5

### Trial to Demonstrate Improved Efficacy of an Anti-Cancer Agent in Subjects with a Modulated Level of Hypoxia

Subjects diagnosed with solid tumors are recruited for a study to determine the efficacy of a selected agent selected from the group consisting of bevacizumab, ganetespib, temsirolimus, erlotinib, PTK787, BEZ235, XL765, pazopanib, cediranib, and axitinib in the treatment of solid tumors, preferably tumors from the same tissue origin, e.g., breast, prostate, lung, liver, brain, colorectal, etc. Inclusion criteria include the presence of a solid tumor and at least 30 days from surgery and any incisions are fully closed. Exclusion criteria include the presence of an ischemia related disease or disorder including, e.g., ischemic heart or vascular disease, poor circulation, diabetes, macular degeneration, recent stroke, or other ischemic events or conditions; or surgery planned during the duration of the trial. Blood and tumor samples are collected for analysis of levels of hypoxia by determining the level of one or more markers of hypoxia, e.g., at least one isoform or subunit of lactate dehydrogenase (LDH) or hypoxia inducible factor (HIF); at least one pro-angiogenic form of vascular endothelial growth factor (VEGF), phosphorylated VEGF receptor (pKDR) 1, 2, or 3; neurolipin 1 (NRP-1), pyruvate dehydrokinase (PDH-K), ornithine decarboxylase (ODC), tumor size. Antibodies against prodrugs that localize in hypoxic regions (e.g., EF5, pimonidazole, etc.) can also be used to detect hypoxia. PET scans can be used to detect hypoxia. Functional imaging measuring blood flow in the tumor can be used as an indicator of hypoxia in the tissue. Direct measurement of hypoxia can be preformed by inserting a sensor into the tumor. Tumor size can also be a marker for hypoxia. Depending on the tumor site, other subject samples can be collected, e.g., fecal matter in subjects with colorectal cancer, urine for subjects with kidney or bladder cancer, cerebrospinal fluid in subjects with brain cancer, etc. by assaying the same markers. Additional samples for analysis can be collected during the course of the study. Complete medical histories are also obtained when not otherwise available.

All subjects are treated with the selected agent selected from the group consisting of bevacizumab, ganetespib, temsirolimus, erlotinib, PTK787, BEZ235, XL765, pazopanib, cediranib, and axitinib, either alone or in combination with one or more additional chemotherapeutic agents. The number regimens used will depend on the size of the study, the number of subjects available, the time frame of the study, etc. The number of regimens is selected to allow the study to be sufficiently powered to provide meaningful results. Subjects are monitored for response to the agent throughout the trial, at the end of the trial, and at regular intervals after the conclusion of the trial using routine methods including, but not limited to, e.g., imaging, hematology, and physical examination. Treatment may be discontinued for non-responsive subjects or for with intolerable side effects. Preferably, the subjects continue to be monitored for outcomes beyond the formal end of the trial. Subjects with a positive response to the treatment regimen can be continued on the regimen beyond the predetermined treatment window of the trial at the discretion of the attending physician.

An analysis of the samples collected from subjects prior to and optionally during treatment is performed to determine the efficacy of the selected agent for the treatment of cancer based on the level of hypoxia of the tumor based on markers assessed prior to and optionally during the treatment of the subjects. The analysis can be performed at the conclusion of the trial, or the analysis can be performed prior to the conclusion of the trial with the results being blinded or not disclosed to the treating physicians. Preferably, the analysis for hypoxia level is determined during the course of the trial to insure that a sufficient number of subjects with high and low hypoxia levels were enrolled in the study to allow for sufficient power of the study to provide a conclusive outcome.

Outcomes of subjects based on the level of hypoxia are analyzed to determine if the outcome between the two groups is different. Outcomes can further be compared to a non-stratified group treated with the agent, e.g., a historical group provided by another study. Samples can be analyzed to confirm the correlation of the level of hypoxia in the tumor to the level of hypoxia in the peripherally collected sample (e.g., blood, urine, cerebrospinal fluid). Methods for statistical analysis and determination of statistical significance are within the ability of those of skill in the art. The analysis demonstrates that subjects with a high level of hypoxia have a better response, e.g., one or more of longer time to failure, longer survival time, better quality of life, decreased tumor size, better tolerance of the selected agent, etc., as compared to subjects with a low level of hypoxia, and that such agents should be preferentially used in subjects having high levels of markers of hypoxia.

### Example 6

### Characterization of Treatment Outcomes to Demonstrate Improved Efficacy of Bevacizumab in Subjects with Colorectal Cancer with a High Level of LDH

Multiple clinical trials have been performed to demonstrate the efficacy of bevacizumab in the treatment of colorectal cancer. For example, a randomized Phase II study was performed to test bevacizumab plus 5-FU/Leucovorin chemotherapy compared to 5-FU/Leucovorin alone in 209 patients who were not optimal candidates to receive first-line Camptothecin-11 (CPT-11) chemotherapy because of age or performance status. The study showed a 29 percent improvement in the primary endpoint of median survival from 16.6 months in the bevacizumab and chemotherapy arm to 12.9 months in the chemotherapy arm. Although this improvement was not statistically significant, it was clinically meaningful for patients with metastatic colorectal cancer and consistent with the results of the pivotal bevacizumab trial.

Further, a combined analysis of the pivotal Phase III trial and two Phase II trials in metastatic colorectal cancer evaluated the safety and efficacy of bevacizumab in combination with 5-FU/Leucovorin chemotherapy (n=249). These results were compared to a combined control group that included patients receiving either 5-fluorouracil (5-FU)/Leucovorin (folinic acid) or the IFL chemotherapy regimen (5-FU/Leucovorin/CPT-11) alone (n=241). Results of this analysis showed that patients receiving bevacizumab and 5-FU/Leucovorin achieved a median survival of 17.9 months compared to 14.6 months in patients receiving the IFL regimen alone. Progression-free survival for patients treated with bevacizumab plus 5-FU/Leucovorin was 8.7 months compared to 5.5 months for patients treated with the IFL regimen alone.

A chart review is performed to determine if levels of one or more hypoxic markers, particularly LDH, were analyzed for the subjects prior to, and optionally during treatment with bevacizumab. If no information is available regarding the levels of hypoxic markers, serum samples retained from the study subjects are analyzed for LDH level and outcomes are analyzed in view of the LDH level.

Preliminarily, subjects within each of the groups, or at least the groups in which subjects were treated with bevacizumab, are divided into high and low LDH level based on the upper limit of normal (ULN) for the site where the testing is done. A value equal to or less than the ULN is considered as low. Values greater than the ULN are considered high. Alternatively, low LDH can be considered as levels up to and including 0.8 ULN with high LDH being considered all values above 0.8 ULN. Alternatively, low LDH can be considered as levels up to and including 1.2 or 1.5 ULN with high LDH being considered all values above 1.2 or 1.5 ULN, respectively. It may be possible to further stratify the high and low ULN groups to provide further predictive power of the LDH level in predicting the response of a subject to treatment with bevacizumab, e.g., assigning those with an LDH level of 1 to <2 times, or 1 to <3 times, etc. the ULN as having an intermediate or slightly elevated LDH level. Ratios of LDH isoforms or subunits, e.g., ratios of the ULN values of LDHA to LDHB or LDH4 and/or LDH5 to LDH1 or total LDH can also be used to determine high and low levels of hypoxia. Other cut-off values such as those provided in the instant application can also be selected. Statistical analysis can be used to select appropriate cut-offs. The outcome of the analysis is further used to select treatment regimens for subjects including or not including bevacizumab based on the ULN level. The outcome of the analysis is further used to allow for the selection of subjects likely to benefit from treatment with bevacizumab based on the ULN level. Subjects with a high level of LDH are selected for treatment with bevacizumab as they are likely to benefit from such treatment. Subjects with a low level of LDH are selected against for treatment with bevacizumab as they are not likely to benefit from such treatment.

### Example 7

### Characterization of Treatment Outcomes to Demonstrate Improved Efficacy of Bevacizumab in Subjects with Pancreatic Cancer with a High Level of LDH

Clinical trials have been performed to demonstrate the efficacy of bevacizumab in the treatment of pancreatic cancer. For example, in one study, 45 patients with metastatic pancreatic cancer received treatment with bevacizumab plus gemcitabine chemotherapy. At the time of analysis, 42 patients were evaluated for response. In the study, the estimated one-year survival was 54 percent and the median time to disease progression was 5.8 months. The results suggest that 21 percent of patients (9/42) experienced a partial response to treatment lasting a median of 9.4 months and 45 percent of patients (19/42) achieved stable disease lasting a median of 5.4 months. Median survival was nine months.

In a second phase III clinical trial of bevacizumab in combination with gemcitabine and erlotinib with patients with metastatic pancreatic cancer, 301 patients received placebo in combination with gemcitabine and erlotinib, while 306 patients received bevacizumab, gemcitabine and erlotinib. Median overall survival was 7.1 and 6.0 months in the bevacizumab and placebo arms, respectively (hazard ratio 0.89, 95% CI, 0.74 to 1.07, p=0.2087), and adding bevacizumab significantly proved progression free survival (HR, 0.73, 95% CI, 0.61 to 0.96; P=0.0002). (See, e.g., Van Cutsem et al., J. Clin. Oncol., 27(13):2231-2237, 2009.)

A chart review is performed to determine if levels of one or more hypoxic markers, particularly LDH, were analyzed for the subjects prior to, and optionally during treatment with bevacizumab. If no information is available regarding the levels of hypoxic markers, serum samples retained from the study subjects are analyzed for LDH level and outcomes are analyzed in view of the LDH level.

Preliminarily, subjects within each of the groups, or at least the groups in which subjects were treated with bevacizumab, are divided into high and low LDH level based on the upper limit of normal (ULN) for the site where the testing is done. A value equal to or less than the ULN is considered as low. Values greater than the ULN are considered high. Alternatively, low LDH can be considered as levels up to and including 0.8 ULN with high LDH being considered all values above 0.8 ULN. Alternatively, low LDH can be considered as levels up to and including 1.2 or 1.5 ULN with high LDH being considered all values above 1.2 or 1.5 ULN, respectively. It may be possible to further stratify the high and low ULN groups to provide further predictive power of the LDH level in predicting the response of a subject to treatment with bevacizumab, e.g., assigning those with an LDH level of 1 to <2 times, or 1 to <3 times, etc. the ULN as having an intermediate or slightly elevated LDH level. Ratios of LDH isoforms or subunits, e.g., ratios of the ULN values of LDHA to LDHB or LDH4 and/or LDH5 to LDH1 or total LDH can also be used to determine high and low levels of hypoxia. Other cut-off values such as those provided in the instant application can also be selected. Statistical analysis can be used to select appropriate cut-offs. The outcome of the analysis is further used to select treatment regimens for subjects including or not including bevacizumab based on the ULN level. The outcome of the analysis is further used to allow for the selection of subjects likely to benefit from treatment with bevacizumab based on the ULN level. Subjects with a high level of LDH are selected for treatment with bevacizumab as they are likely to benefit from such treatment. Subjects with a low level of LDH are selected against for treatment with bevacizumab as they are not likely to benefit from such treatment.

### Example 8

### Characterization of Treatment Outcomes to Demonstrate Improved Efficacy of Bevacizumab in Subjects with Lung Cancer with a High Level of LDH

Clinical trials have been performed to demonstrate the efficacy of bevacizumab in the treatment of lung cancer. For example, in one study, A total of 878 patients with advanced non-squamous, non-small cell lung cancer (NSCLC) who had not previously received systemic chemotherapy were enrolled in this study between July 2001 and April 2004. Patients were randomized to one of the two treatment arms. One patient group received standard treatment—six cycles of paclitaxel and carboplatin. The second group received the same six-cycle chemotherapy regimen with the addition of bevacizumab, followed by bevacizumab alone until disease progression. Patients with squamous cell carcinoma of the lung were not included in the study because previous clinical experience suggested that patients with this particular type of NSCLC had a higher risk of serious bleeding from the lung after bevacizumab therapy. Patients with a prior history of frank hemoptysis (coughing up blood) were also not enrolled on the trial.

Researchers found that patients in the study who received bevacizumab in combination with standard chemotherapy (a treatment regimen of paclitaxel and carboplatin) had a median overall survival of 12.5 months compared to patients treated with the standard chemotherapy alone, who had a median survival of 10.2 months. This difference was found to be statistically significant.

A chart review is performed to determine if levels of one or more hypoxic markers, particularly LDH, were analyzed for the subjects prior to, and optionally during treatment with bevacizumab. If no information is available regarding the levels of hypoxic markers, serum samples retained from the study subjects are analyzed for LDH level and outcomes are analyzed in view of the LDH level.

Preliminarily, subjects within each of the groups, or at least the groups in which subjects were treated with bevacizumab, are divided into high and low LDH level based on the upper limit of normal (ULN) for the site where the testing is done. A value equal to or less than the ULN is considered as low. Values greater than the ULN are considered high. Alternatively, low LDH can be considered as levels up to and including 0.8 ULN with high LDH being considered all values above 0.8 ULN. Alternatively, low LDH can be considered as levels up to and including 1.2 or 1.5 ULN with high LDH being considered all values above 1.2 or 1.5 ULN, respectively. It may be possible to further stratify the high and low ULN groups to provide further predictive power of the LDH level in predicting the response of a subject to treatment with bevacizumab, e.g., assigning those with an LDH level of 1 to <2 times, or 1 to <3 times, etc. the ULN as having an intermediate or slightly elevated LDH level. Ratios of LDH isoforms or subunits, e.g., ratios of the ULN values of LDHA to LDHB or LDH4 and/or LDH5 to LDH1 or total LDH can also be used to determine high and low levels of hypoxia. Other cut-off values such as those provided in the instant application can also be selected. Statistical analysis can be used to select appropriate cut-offs. The outcome of the analysis is further used to select treatment regimens for subjects including or not including bevacizumab based on the ULN level. The outcome of the analysis is further used to allow for the selection of subjects likely to benefit from treatment with bevacizumab based on the ULN level. Subjects with a high level of LDH are selected for treatment with bevacizumab as they are likely to benefit from such treatment.

Subjects with a low level of LDH are selected against for treatment with bevacizumab as they are not likely to benefit from such treatment.

### Example 9

### Characterization of Treatment Outcomes to Demonstrate Improved Efficacy of Bevacizumab in Subjects with Glioblastoma with a High Level of LDH

Clinical trials have been performed to demonstrate the efficacy of bevacizumab in the treatment of brain cancer, particularly glioblastoma multiforme (GBM). Glioblastomas are fast-growing brain tumors that can invade normal brain tissue, which can make them very difficult to treat. Two phase II clinical trials that showed bevacizumab reduced tumor size in some glioblastoma patients. The first study split 167 patients into 2 groups: one group received bevacizumab alone; the other a combination of bevacizumab and the chemotherapy drug irinotecan. Of the 85 patients treated with bevacizumab alone, 26% had their tumors shrink in response to the drug. In the second trial, which followed 56 patients who were treated with bevacizumab alone, 20% responded to the drug. In both studies, the effect lasted for an average of about 4 months.

A chart review is performed to determine if levels of one or more hypoxic markers, particularly LDH, were analyzed for the subjects prior to, and optionally during treatment with bevacizumab. If no information is available regarding the levels of hypoxic markers, serum samples retained from the study subjects are analyzed for LDH level and outcomes are analyzed in view of the LDH level.

Preliminarily, subjects within each of the groups, or at least the groups in which subjects were treated with bevacizumab, are divided into high and low LDH level based on the upper limit of normal (ULN) for the site where the testing is done. A value equal to or less than the ULN is considered as low. Values greater than the ULN are considered high. Alternatively, low LDH can be considered as levels up to and including 0.8 ULN with high LDH being considered all values above 0.8 ULN. Alternatively, low LDH can be considered as levels up to and including 1.2 or 1.5 ULN with high LDH being considered all values above 1.2 or 1.5 ULN, respectively. It may be possible to further stratify the high and low ULN groups to provide further predictive power of the LDH level in predicting the response of a subject to treatment with bevacizumab, e.g., assigning those with an LDH level of 1 to <2 times, or 1 to <3 times, etc. the ULN as having an intermediate or slightly elevated LDH level. Ratios of LDH isoforms or subunits, e.g., ratios of the ULN values of LDHA to LDHB or LDH4 and/or LDH5 to LDH1 or total LDH can also be used to determine high and low levels of hypoxia. Other cut-off values such as those provided in the instant application can also be selected. Statistical analysis can be used to select appropriate cut-offs. The outcome of the analysis is further used to select treatment regimens for subjects including or not including bevacizumab based on the ULN level. The outcome of the analysis is further used to allow for the selection of subjects likely to benefit from treatment with bevacizumab based on the ULN level. Subjects with a high level of LDH are selected for treatment with bevacizumab as they are likely to benefit from such treatment. Subjects with a low level of LDH are selected against for treatment with bevacizumab as they are not likely to benefit from such treatment.

### Example 10

### Characterization of Treatment Outcomes to Demonstrate Improved Efficacy of Bevacizumab in Subjects with Renal Cancer with a High Level of LDH

Clinical trials have been performed to demonstrate the efficacy of bevacizumab in the treatment of renal cancer. A phase III study found the drug combination of interferon-alpha, the standard of care, with bevacizumab, increased progression-free survival time by about 5 months compared to taking interferon-alpha alone. Tumor size decreased in 30% of patients taking the bevacizumab and interferon-alpha combination, compared to just 12% of patients taking interferon-alpha alone.

A chart review is performed to determine if levels of one or more hypoxic markers, particularly LDH, were analyzed for the subjects prior to, and optionally during treatment with bevacizumab. If no information is available regarding the levels of hypoxic markers, serum samples retained from the study subjects are analyzed for LDH level and outcomes are analyzed in view of the LDH level.

Preliminarily, subjects within each of the groups, or at least the groups in which subjects were treated with bevacizumab, are divided into high and low LDH level based on the upper limit of normal (ULN) for the site where the testing is done. A value equal to or less than the ULN is considered as low. Values greater than the ULN are considered high. Alternatively, low LDH can be considered as levels up to and including 0.8 ULN with high LDH being considered all values above 0.8 ULN. Alternatively, low LDH can be considered as levels up to and including 1.2 or 1.5 ULN with high LDH being considered all values above 1.2 or 1.5 ULN, respectively. It may be possible to further stratify the high and low ULN groups to provide further predictive power of the LDH level in predicting the response of a subject to treatment with bevacizumab, e.g., assigning those with an LDH level of 1 to <2 times, or 1 to <3 times, etc. the ULN as having an intermediate or slightly elevated LDH level. Ratios of LDH isoforms or subunits, e.g., ratios of the ULN values of LDHA to LDHB or LDH4 and/or LDH5 to LDH1 or total LDH can also be used to determine high and low levels of hypoxia. Other cut-off values such as those provided in the instant application can also be selected. Statistical analysis can be used to select appropriate cut-offs. The outcome of the analysis is further used to select treatment regimens for subjects including or not including bevacizumab based on the ULN level. The outcome of the analysis is further used to allow for the selection of subjects likely to benefit from treatment with bevacizumab based on the ULN level. Subjects with a high level of LDH are selected for treatment with bevacizumab as they are likely to benefit from such treatment. Subjects with a low level of LDH are selected against for treatment with bevacizumab as they are not likely to benefit from such treatment.

### Example 11

### Characterization of Treatment Outcomes to Demonstrate Improved Efficacy of Bevacizumab in Subjects with Breast Cancer with a High Level of LDH

Clinical trials have been performed to demonstrate the efficacy of bevacizumab in the treatment of breast cancer. After an initial approval of the use of bevacizumab for the treatment of breast cancer in conjunction with other agents, the Oncologic Drugs Advisory Committee voted that bevacizumab when added to standard chemotherapy does not keep cancer from worsening for a long enough time to be clinically meaningful to HER2-negative, metastatic breast cancer. The FDA withdrew approval of the drug for the treatment of breast cancer. However, initial approval of the drug for treatment of breast cancer demonstrates that a group of the subjects were found to benefit from treatment with bevacizumab.

A chart review is performed to determine if levels of one or more hypoxic markers, particularly LDH, were analyzed for the subjects prior to, and optionally during treatment with bevacizumab. If no information is available regarding the levels of hypoxic markers, serum samples retained from the study subjects are analyzed for LDH level and outcomes are analyzed in view of the LDH level.

Preliminarily, subjects within each of the groups, or at least the groups in which subjects were treated with bevacizumab, are divided into high and low LDH level based on the upper limit of normal (ULN) for the site where the testing is done. A value equal to or less than the ULN is considered as low. Values greater than the ULN are considered high. Alternatively, low LDH can be considered as levels up to and including 0.8 ULN with high LDH being considered all values above 0.8 ULN. Alternatively, low LDH can be considered as levels up to and including 1.2 or 1.5 ULN with high LDH being considered all values above 1.2 or 1.5 ULN, respectively. It may be possible to further stratify the high and low ULN groups to provide further predictive power of the LDH level in predicting the response of a subject to treatment with bevacizumab, e.g., assigning those with an LDH level of 1 to <2 times, or 1 to <3 times, etc. the ULN as having an intermediate or slightly elevated LDH level. Ratios of LDH isoforms or subunits, e.g., ratios of the ULN values of LDHA to LDHB or LDH4 and/or LDH5 to LDH1 or total LDH can also be used to determine high and low levels of hypoxia. Other cut-off values such as those provided in the instant application can also be selected. Statistical analysis can be used to select appropriate cut-offs. The outcome of the analysis is further used to select treatment regimens for subjects including or not including bevacizumab based on the ULN level. The outcome of the analysis is further used to allow for the selection of subjects likely to benefit from treatment with bevacizumab based on the ULN level. Subjects with a high level of LDH are selected for treatment with bevacizumab as they are likely to benefit from such treatment. Subjects with a low level of LDH are selected against for treatment with bevacizumab as they are not likely to benefit from such treatment.

### Example 12

### Trial to Demonstrate Improved Efficacy of Bevacizumab in Subjects with Various Cancer Types with a High Level of LDH

Subjects are identified as having one of colorectal cancer, lung cancer, breast cancer, brain cancer, or renal cell carcinoma. A subject is selected as being candidate for treatment with bevacizumab based on sufficient hepatic function and having no recent wounds or risks for bleeding disorders, particularly gastrointestinal bleeding. Routine assessments are made prior to treatment to characterize the disease state of the subject including, but not limited to, imaging studies, hematological studies, and physical examination. Additionally, coded serum sample from the subject is tested to determine the LDH level. The results from the LDH level determination are not matched to the subject until the end of the treatment period. However, samples can be tested to allow sufficient numbers of subjects with low and high LDH levels to be recruited to provide sufficient power to the study.

Subjects are treated with the standard dose of bevacizumab, either alone or in combination with other agents. For example, the following protocols can be used for various cancer types:

Metastatic Colorectal Cancer (mCRC)

The recommended doses are 5 mg/kg or 10 mg/kg every 2 weeks when used in combination with intravenous 5-FU-based chemotherapy.

Administer 5 mg/kg when used in combination with bolus-IFL.

Administer 10 mg/kg when used in combination with FOLFOX4.

### Non-Squamous Non-Small Cell Lung Cancer (NSCLC)

The recommended dose is 15 mg/kg every 3 weeks in combination with carboplatin and paclitaxel.

### Metastatic Breast Cancer (MBC)

The recommended dose is 10 mg/kg every 2 weeks in combination with paclitaxel.

### Glioblastoma

The recommended dose is 10 mg/kg every 2 weeks.

Metastatic Renal Cell Carcinoma (mRCC)

The recommended dose is 10 mg/kg every 2 weeks in combination with interferon alpha.

At predetermined regular or irregular intervals, subjects are assessed for specific outcomes including, but not limited to, overall survival, progression free survival, time to progression, and adverse events. Treatment is continued for as long as the subject responds positively to treatment with bevacizumab and there are no limiting adverse events.

Upon conclusion of the study, the results from the LDH level analysis are unblinded and matched to the subjects. As specific methods of testing are available, the amount of LDH is scored as being low or high based on the upper limit of normal (ULN) for the site where the testing is done. A value equal to or less than the ULN is considered as low. Values greater than the ULN are considered high. Alternatively, low LDH can be considered as levels up to and including 0.8 ULN with high LDH being considered all values above 0.8 ULN. Alternatively, low LDH can be considered as levels up to and including 1.2 or 1.5 ULN with high LDH being considered all values above 1.2 or 1.5 ULN, respectively. It may be possible to further stratify the high and low ULN groups to provide further predictive power of the LDH level in predicting the response of a subject to treatment with temsirolimus, e.g., assigning those with an LDH level of 1 to <2 times, or 1 to <3 times, etc. the ULN as having an intermediate or slightly elevated LDH level. Ratios of LDH isoforms or subunits, e.g., ratios of the ULN values of LDHA to LDHB or LDH4 and/or LDH5 to LDH1 or total LDH can also be used to determine high and low levels of hypoxia. Other cut-off values such as those provided in the instant application can also be selected. Statistical analysis can be used to select appropriate cut-offs. The outcome of the analysis is further used to select treatment regimens for subjects including or not including bevacizumab based on the ULN level. The outcome of the analysis is further used to allow for the selection of subjects likely to benefit from treatment with bevacizumab based on the ULN level. Subjects with a high level of LDH are selected for treatment with bevacizumab as they are likely to benefit from such treatment. Subjects with a low level of LDH are selected against for treatment with bevacizumab as they are not likely to benefit from such treatment.

### Example 13

### Selection of Subjects with Colon Cancer and a High Level of LDH for Treatment with Bevacizumab

Subject is identified as having colon cancer, particularly metastatic colon cancer, or other cancer type known to be or suspected to be susceptible to treatment with bevacizumab, and being candidate for treatment with bevacizumab. A serum sample from the subject is tested to determine the LDH level. The amount of LDH is scored as being low or high based on the upper limit of normal (ULN) for the site where the testing is done. A value equal to or less than the ULN is considered as low. A value greater than the ULN is considered to be high. Alternatively, low LDH can be considered as levels up to and including 0.8 ULN with high LDH being considered all values above 0.8 ULN. Alternatively, low LDH can be considered as levels up to and including 1.2 or 1.5 ULN with high LDH being considered all values above 1.2 or 1.5 ULN, respectively. It may be possible to further stratify the high and low ULN groups to provide further predictive power of the LDH level in predicting the response of a subject to treatment with temsirolimus, e.g., assigning those with an LDH level of 1 to <2 times, or 1 to <3 times, etc. the ULN as having an intermediate or slightly elevated LDH level. Ratios of LDH isoforms or subunits, e.g., ratios of the ULN values of LDHA to LDHB or LDH4 and/or LDH5 to LDH1 or total LDH can also be used to determine high and low levels of hypoxia. Other cut-off values such as those provided in the instant application can also be selected.

If the subject has a low LDH level, treatment with compounds other than bevacizumab is selected. If the subject has a high LDH level, treatment with bevacizumab, optionally with other agents, is selected as the treatment regimen.

### Example 14

### Characterization of Treatment Outcomes to Demonstrate Improved Efficacy of Ganetespib in Subjects with Solid Tumors with a High Level of LDH

Clinical trials have been performed to demonstrate the efficacy of ganetespib in the treatment of cancer. For example, a Phase 1 study was performed to test ganetespib with a once-weekly dosing scheduling for the treatment of solid tumors. The study demonstrated that several patients who had progressed or failed to respond to multiple prior therapies experienced substantial tumor shrinkage and prolonged disease control with ganetespib and that more than half of all evaluable patients experienced disease control. In another Phase 1 study, over half the patients in this heavily pre-treated population received at least 4 cycles of treatment consisting of ganetespib at a dose of 150 mg/m² in combination with docetaxel at a dose of 75 mg/m² demonstrating the safety of the dosing regimen. From the study, a confirmed partial response, with over 50% shrinkage of target tumor lesions, was reported for a patient on the trial diagnosed with cancer of the parotid gland, the largest of the salivary glands. The patient did not respond to prior treatment regimens including carboplatin, cetuximab, and methotrexate. A Phase ½ clinical trial was performed to demonstrate the greater efficacy of ganetespib in combination with 5FU and radiation in reducing rectal cancer cell colony formation more than 5 FU and radiation alone. Results from a Phase 2 single agent NSCLC trial showed that ganetespib had a 54% disease control rate in the broad population of patients in the trial with advanced relapsed/refractory NSCLC, all of whom had progressive disease upon study entry. In addition, six of eight patients (75%) with ALK rearrangement experienced tumor shrinkage, including four patients (50%) with durable, objective responses. Seven of eight of these patients (88%) received ganetespib for 16 weeks or more. Tumor shrinkage also occurred in 62% of patients whose tumors have a KRAS mutation, a particularly therapeutically challenging population. ganetespib was well tolerated in this study and did not have the serious hepatic or common ocular toxicities reported with other Hsp90 inhibitors. The favorable safety profile seen in this trial is consistent with results seen in over 15 trials initiated to date with nearly 400 patients treated. Further studies are ongoing.

A chart review is performed to determine if levels of one or more hypoxic markers, particularly LDH, is analyzed for the subjects prior to, and optionally during treatment with ganetespib. If no information is available regarding the levels of hypoxic markers, serum samples retained from the study subjects are analyzed for LDH level and outcomes are analyzed in view of the LDH level.

Preliminarily, subjects within each of the groups, or at least the groups in which subjects were treated with ganetespib, are divided into high and low LDH level based on the upper limit of normal (ULN) for the site where the testing is done. A value equal to or less than the ULN is considered as low. Values greater than the ULN are considered high. Alternatively, low LDH can be considered as levels up to and including 0.8 ULN with high LDH being considered all values above 0.8 ULN. Alternatively, low LDH can be considered as levels up to and including 1.2 or 1.5 ULN with high LDH being considered all values above 1.2 or 1.5 ULN, respectively. It may be possible to further stratify the high and low ULN groups to provide further predictive power of the LDH level in predicting the response of a subject to treatment with ganetespib, e.g., assigning those with an LDH level of 1 to <2 times, or 1 to <3 times, etc. the ULN as having an intermediate or slightly elevated LDH level. Ratios of LDH isoforms or subunits, e.g., ratios of the ULN values of LDHA to LDHB or LDH4 and/or LDH5 to LDH1 or total LDH can also be used to determine high and low levels of hypoxia. Other cut-off values such as those provided in the instant application can also be selected. Statistical analysis can be used to select appropriate cut-offs. The outcome of the analysis is further used to select treatment regimens for subjects including or not including ganetespib based on the ULN level. The outcome of the analysis is further used to allow for the selection of subjects likely to benefit from treatment with ganetespib based on the ULN level. Subjects with a high level of LDH are selected for treatment with ganetespib as they are likely to benefit from such treatment. Subjects with a low level of LDH are selected against for treatment with ganetespib as they are not likely to benefit from such treatment.

### Example 15

### Characterization of Treatment Outcomes to Demonstrate Improved Efficacy of Ganetespib in Subjects with Other Cancers with Other Cancers with a High Level of LDH

Multiple Phase 1 and 2 clinical trials have been and are being performed to demonstrate the efficacy of ganetespib in non-small cell lung cancer, gastrointestinal stromal tumors, colorectal cancer, gastric cancer, small cell lung cancer, and melanoma as discussed in the previous example.

A chart review is performed to determine if levels of one or more hypoxic markers, particularly LDH, is analyzed for the subjects prior to, and optionally during treatment with ganetespib. If no information is available regarding the levels of hypoxic markers, serum samples retained from the study subjects are analyzed for LDH level and outcomes are analyzed in view of the LDH level.

Preliminarily, subjects within each of the groups, or at least the groups in which subjects were treated with ganetespib, are divided into high and low LDH level based on the upper limit of normal (ULN) for the site where the testing is done. A value equal to or less than the ULN is considered as low. Values greater than the ULN are considered high. Alternatively, low LDH can be considered as levels up to and including 0.8 ULN with high LDH being considered all values above 0.8 ULN. Alternatively, low LDH can be considered as levels up to and including 1.2 or 1.5 ULN with high LDH being considered all values above 1.2 or 1.5 ULN, respectively. It may be possible to further stratify the high and low ULN groups to provide further predictive power of the LDH level in predicting the response of a subject to treatment with ganetespib, e.g., assigning those with an LDH level of 1 to <2 times, or 1 to <3 times, etc. the ULN as having an intermediate or slightly elevated LDH level. Ratios of LDH isoforms or subunits, e.g., ratios of the ULN values of LDHA to LDHB or LDH4 and/or LDH5 to LDH1 or total LDH can also be used to determine high and low levels of hypoxia. Other cut-off values such as those provided in the instant application can also be selected. Statistical analysis can be used to select appropriate cut-offs. The outcome of the analysis is further used to select treatment regimens for subjects including or not including ganetespib based on the ULN level. The outcome of the analysis is further used to allow for the selection of subjects likely to benefit from treatment with ganetespib based on the ULN level. Subjects with a high level of LDH are selected for treatment with ganetespib as they are likely to benefit from such treatment. Subjects with a low level of LDH are selected against for treatment with ganetespib as they are not likely to benefit from such treatment.

### Example 16

### Trial to Demonstrate Improved Efficacy of Ganetespib in Subjects with Various Cancer Types with a High Level of LDH

Subjects are identified as having one of advanced solid tumor malignancies including metastatic or unresectable malignancy with evidence of progression, non-small cell lung cancer, gastrointestinal stromal tumors, colorectal cancer, gastric cancer, small cell lung cancer, melanoma, refractory malignancy. A subject is selected as being candidate for treatment with ganetespib. Routine assessments are made prior to treatment to characterize the disease state of the subject including, but not limited to, imaging studies, hematological studies, and physical examination. Additionally, coded serum sample from the subject is tested to determine the LDH level. The results from the LDH level determination are not matched to the subject until the end of the treatment period. However, samples can be tested to allow sufficient numbers of subjects with low and high LDH levels to be recruited to provide sufficient power to the study.

Subjects are treated with the standard dose of ganetespib, either alone or in combination with other agents, e.g., using the regimens presented in the prior examples. At predetermined regular or irregular intervals, subjects are assessed for specific outcomes including, but not limited to, overall survival, progression free survival, time to progression, and adverse events. Treatment is continued for as long as the subject responds positively to treatment with bevacizumab and there are no limiting adverse events.

Upon conclusion of the study, the results from the LDH level analysis are unblinded and matched to the subjects. As specific methods of testing are available, the amount of LDH is scored as being low or high based on the upper limit of normal (ULN) for the site where the testing is done. A value equal to or less than the ULN is considered as low. Values greater than the ULN are considered high. Alternatively, low LDH can be considered as levels up to and including 0.8 ULN with high LDH being considered all values above 0.8 ULN. Alternatively, low LDH can be considered as levels up to and including 1.2 or 1.5 ULN with high LDH being considered all values above 1.2 or 1.5 ULN, respectively. It may be possible to further stratify the high and low ULN groups to provide further predictive power of the LDH level in predicting the response of a subject to treatment with ganetespib, e.g., assigning those with an LDH level of 1 to <2 times, or 1 to <3 times, etc. the ULN as having an intermediate or slightly elevated LDH level. Ratios of LDH isoforms or subunits, e.g., ratios of the ULN values of LDHA to LDHB or LDH4 and/or LDH5 to LDH1 or total LDH can also be used to determine high and low levels of hypoxia. Other cut-off values such as those provided in the instant application can also be selected. Statistical analysis can be used to select appropriate cut-offs. The outcome of the analysis is further used to select treatment regimens for subjects including or not including ganetespib based on the ULN level. The outcome of the analysis is further used to allow for the selection of subjects likely to benefit from treatment with ganetespib based on the ULN level. Subjects with a high level of LDH are selected for treatment with ganetespib as they are likely to benefit from such treatment. Subjects with a low level of LDH are selected against for treatment with ganetespib as they are not likely to benefit from such treatment.

### Example 17

### Selection of Subjects with Lung Cancer and a High Level of LDH for Treatment with Ganetespib

Subject is identified as having lung cancer, either small cell or non-small cell lung cancer, or other cancer type known to be or suspected to be susceptible to treatment with ganetespib, and being candidate for treatment with ganetespib. A serum sample from the subject is tested to determine the LDH level. The amount of LDH is scored as being low or high based on the upper limit of normal (ULN) for the site where the testing is done. A value equal to or less than the ULN is considered as low. A value greater than the ULN is considered to be high. Alternatively, low LDH can be considered as levels up to and including 0.8 ULN with high LDH being considered all values above 0.8 ULN. Alternatively, low LDH can be considered as levels up to and including 1.2 or 1.5 ULN with high LDH being considered all values above 1.2 or 1.5 ULN, respectively. It may be possible to further stratify the high and low ULN groups to provide further predictive power of the LDH level in predicting the response of a subject to treatment with temsirolimus, e.g., assigning those with an LDH level of 1 to <2 times, or 1 to <3 times, etc. the ULN as having an intermediate or slightly elevated LDH level. Ratios of LDH isoforms or subunits, e.g., ratios of the ULN values of LDHA to LDHB or LDH4 and/or LDH5 to LDH1 or total LDH can also be used to determine high and low levels of hypoxia. Other cut-off values such as those provided in the instant application can also be selected.

If the subject has a low LDH level, treatment with compounds other than ganetespib is selected. If the subject has a high LDH level, treatment with ganetespib, optionally with other agents, is selected as the treatment regimen.

### Example 18

### Characterization of Treatment Outcomes to Demonstrate Improved Efficacy of Temsirolimus in Subjects with Renal Cancer with a High Level of LDH

Clinical trials have been performed to demonstrate the efficacy of temsirolimus in the treatment of renal cancer, particularly advanced renal cell carcinoma (RCC). A three-arm, phase 3 clinical trial of 626 patients with advanced RCC and poor prognosis who had received no prior systemic therapy was performed to compare the efficacy of temsirolimus alone as compared to interferon (IFN)-α, the standard of care, and a combination of temsirolimus and IFN-α. Temsirolimus significantly increased median overall survival by 49 percent compared to interferon-alpha (10.9 months vs. 7.3 months, P=0.0078). Temsirolimus also was associated with a statistically significant improvement over interferon-alpha in the secondary endpoint of progression-free survival (5.5 months vs. 3.1 months, P=0.0001). However, the combination of temsirolimus and interferon-alpha did not result in a significant increase in overall survival when compared with interferon-alpha alone.

A chart review is performed to determine if levels of one or more hypoxic markers, particularly LDH, were analyzed for the subjects prior to, and optionally during treatment with temsirolimus. If no information is available regarding the levels of hypoxic markers, serum samples retained from the study subjects are analyzed for LDH level and outcomes are analyzed in view of the LDH level.

Preliminarily, subjects within each of the groups, or at least the groups in which subjects were treated with temsirolimus, are divided into high and low LDH level based on the upper limit of normal (ULN) for the site where the testing is done. A value equal to or less than the ULN is considered as low. Values greater than the ULN are considered high. Alternatively, low LDH can be considered as levels up to and including 0.8 ULN with high LDH being considered all values above 0.8 ULN. Alternatively, low LDH can be considered as levels up to and including 1.2 or 1.5 ULN with high LDH being considered all values above 1.2 or 1.5 ULN, respectively. It may be possible to further stratify the high and low ULN groups to provide further predictive power of the LDH level in predicting the response of a subject to treatment with temsirolimus, e.g., assigning those with an LDH level of 1 to <2 times, or 1 to <3 times, etc. the ULN as having an intermediate or slightly elevated LDH level. Ratios of LDH isoforms or subunits, e.g., ratios of the ULN values of LDHA to LDHB or LDH4 and/or LDH5 to LDH1 or total LDH can also be used to determine high and low levels of hypoxia. Other cut-off values such as those provided in the instant application can also be selected. Statistical analysis can be used to select appropriate cut-offs. The outcome of the analysis is further used to select treatment regimens for subjects including or not including temsirolimus based on the ULN level. The outcome of the analysis is further used to allow for the selection of subjects likely to benefit from treatment with temsirolimus based on the ULN level. Subjects with a high level of LDH are selected for treatment with temsirolimus as they are likely to benefit from such treatment. Subjects with a low level of LDH are selected against for treatment with temsirolimus as they are not likely to benefit from such treatment.

### Example 19

### Characterization of Treatment Outcomes to Demonstrate Improved Efficacy of Temsirolimus in Subjects with Renal Cancer with a High Level of LDH

Clinical trials have been performed to demonstrate the efficacy of temsirolimus in the treatment of renal cancer, particularly advanced renal cell carcinoma (RCC). A phase 3 clinical trial of 404 patients with RCC and poor prognosis was performed to compare the efficacy of temsirolimus alone as compared to interferon (IFN)-α, the standard of care. Mean baseline serum normalized LDH was 1.23 times the upper limit of normal (range 0.05 to 28.5×ULN). Survival was significantly improved in 140 subjects with elevated LDH, while survival was not improved with temsirolimus as compared to interferon therapy (6.9 versus 4.2 months, log-rank p<0.005). The 264 subjects with normal LDH did not exhibit improved survival with temsirolimus as compared to interferon therapy (11.7 versus 10.4 months, log-rank p=0.514).

A statistically significant interaction effect was noted between normalized LDH and treatment group (p=0.031), and the hazard ratio for death was 1.98 (95% confidence interval 1.6-2.5, p<0.0001) for patients with LDH>1×ULN compared to patients whose LDH ≦1 ULN. The HR for death was 2.01 for patients with LDH >1 ULN versus ≦1 ULN (95% confidence interval 1.6-2.6, p<0.0001). Post-treatment LDH at two months increased 1.7% versus 27% in the interferon and temsirolimus arms, respectively. (See, e.g., Armstrong et al., J. Clin. Oncol. 28:15s, 2010 (suppl.; abstr. 4631)).

### Example 20

### Trial to Demonstrate Improved Efficacy of Temsirolimus in Subjects with Renal Cancer with a High Level of LDH

Subjects are identified as having renal cell carcinoma and have not previously been treated with any chemotherapeutic agents. A subject is selected as being candidate for treatment with temsirolimus based on sufficient hepatic function and having no recent wounds or risks for bleeding disorders, particularly gastrointestinal bleeding. Routine assessments are made prior to treatment to characterize the disease state of the subject including, but not limited to, imaging studies, hematological studies, and physical examination. Additionally, coded serum sample from the subject is tested to determine the LDH level. The results from the LDH level determination are not matched to the subject until the end of the treatment period. However, samples can be tested to allow sufficient numbers of subjects with low and high LDH levels to be recruited to provide sufficient power to the study.

Subjects are treated with 25 mg of temsirolimus weekly infused over a period of 30 to 60 minutes. At predetermined regular or irregular intervals, subjects are assessed for specific outcomes including, but not limited to, overall survival, progression free survival, time to progression, and adverse events. Treatment is continued for as long as the subject responds positively to treatment with temsirolimus and there are no limiting adverse events. However, an arbitrary treatment window can be selected to allow for conclusion of the trial. In the event of a transient adverse event, e.g., low platelet count, high neutrophil count, high bilirubin, low liver function, etc., a treatment week can be skipped and treatment resumed the following week if the adverse event has passed.

Upon conclusion of the study, the results from the LDH level analysis are unblinded and matched to the subjects. As specific methods of testing are available, the amount of LDH is scored as being low or high based on the upper limit of normal (ULN) for the site where the testing is done. A value equal to or less than the ULN is considered as low. A value greater than the ULN is considered to be high. Alternatively, low LDH can be considered as levels up to and including 0.8 ULN with high LDH being considered all values above 0.8 ULN. Alternatively, low LDH can be considered as levels up to and including 1.2 or 1.5 ULN with high LDH being considered all values above 1.2 or 1.5 ULN, respectively. It may be possible to further stratify the high and low ULN groups to provide further predictive power of the LDH level in predicting the response of a subject to treatment with temsirolimus, e.g., assigning those with an LDH level of 1 to <2 times, or 1 to <3 times, etc. the ULN as having an intermediate or slightly elevated LDH level. Ratios of LDH isoforms or subunits, e.g., ratios of the ULN values of LDHA to LDHB or LDH4 and/or LDH5 to LDH1 or total LDH can also be used to determine high and low levels of hypoxia. Other cut-off values such as those provided in the instant application can also be selected. Statistical analysis can be used to select appropriate cut-offs. The outcome of the analysis is further used to select treatment regimens for subjects including or not including temsirolimus based on the ULN level. The outcome of the analysis is further used to allow for the selection of subjects likely to benefit from treatment with temsirolimus based on the ULN level. Subjects with a high level of LDH are selected for treatment with temsirolimus as they are likely to benefit from such treatment. Subjects with a low level of LDH are selected against for treatment with temsirolimus as they are not likely to benefit from such treatment.

### Example 21

### Selection of Subjects with Renal Cancer and a High Level of LDH for Treatment with Temsirolimus

Subject is identified as having renal cell carcinoma, particularly advanced renal cell carcinoma and being candidate for treatment with temsirolimus based on sufficient hepatic function and having no recent wounds or risks for bleeding disorders, particularly gastrointestinal bleeding. A serum sample from the subject is tested to determine the LDH level. The amount of LDH is scored as being low or high based on the upper limit of normal (ULN) for the site where the testing is done. A value equal to or less than the ULN is considered as low. A value greater than the ULN is considered to be high. Alternatively, low LDH can be considered as levels up to and including 0.8 ULN with high LDH being considered all values above 0.8 ULN. Alternatively, low LDH can be considered as levels up to and including 1.2 or 1.5 ULN with high LDH being considered all values above 1.2 or 1.5 ULN, respectively. It may be possible to further stratify the high and low ULN groups to provide further predictive power of the LDH level in predicting the response of a subject to treatment with temsirolimus, e.g., assigning those with an LDH level of 1 to <2 times, or 1 to <3 times, etc. the ULN as having an intermediate or slightly elevated LDH level. Ratios of LDH isoforms or subunits, e.g., ratios of the ULN values of LDHA to LDHB or LDH4 and/or LDH5 to LDH1 or total LDH can also be used to determine high and low levels of hypoxia. Other cut-off values such as those provided in the instant application can also be selected. The outcome of the analysis is further used to select treatment regimens for subjects including or not including temsirolimus based on the ULN level. Subjects with a high level of LDH are selected for treatment with temsirolimus. Subjects with a low level of LDH are selected against for treatment with temsirolimus.

If the subject has a low LDH level, treatment with compounds other than temsirolimus, or treatment with compounds prior to temsirolimus to increase LDH levels is selected. If an agent to increase the level of LDH is given, the LDH level is tested prior to initiation of treatment with temsirolimus. If the subject has a high LDH level, treatment with temsirolimus, optionally with other agents, is selected as the treatment regimen.

### Example 22

### Selection of Subjects with Non-Hodgkin's Lymphoma and a High Level of LDH for Treatment with Temsirolimus

Subject is identified as having B-cell non-Hodgkin's lymphoma, particularly mantle cell lymphoma and being candidate for treatment with temsirolimus based on sufficient hepatic function and having no recent wounds or risks for bleeding disorders, particularly gastrointestinal bleeding. A serum sample from the subject is tested to determine the LDH level. As specific methods of testing are available, the amount of LDH is scored as being low or high based on the upper limit of normal (ULN) for the site where the testing is done. A value equal to or less than the ULN is considered as low. A value greater than the ULN is considered to be high. Alternatively, low LDH can be considered as levels up to and including 0.8 ULN with high LDH being considered all values above 0.8 ULN. Alternatively, low LDH can be considered as levels up to and including 1.2 or 1.5 ULN with high LDH being considered all values above 1.2 or 1.5 ULN, respectively. It may be possible to further stratify the high and low ULN groups to provide further predictive power of the LDH level in predicting the response of a subject to treatment with temsirolimus, e.g., assigning those with an LDH level of 1 to <2 times, or 1 to <3 times, etc. the ULN as having an intermediate or slightly elevated LDH level. Ratios of LDH isoforms or subunits, e.g., ratios of the ULN values of LDHA to LDHB or LDH4 and/or LDH5 to LDH1 or total LDH can also be used to determine high and low levels of hypoxia. Other cut-off values such as those provided in the instant application can also be selected. The outcome of the analysis is further used to select treatment regimens for subjects including or not including temsirolimus based on the ULN level. Subjects with a high level of LDH are selected for treatment with temsirolimus. Subjects with a low level of LDH are selected against for treatment with temsirolimus.

If the subject has a low LDH level, treatment with compounds other than temsirolimus, or treatment with compounds prior to temsirolimus to increase LDH levels is selected. If an agent to increase the level of LDH is given, the LDH level is tested prior to initiation of treatment with temsirolimus. If the subject has a high LDH level, treatment with temsirolimus, optionally with other agents, is selected as the treatment regimen.

### Example 23

### Characterization of Treatment Outcomes to Demonstrate Improved Efficacy of Erlotinib in Subjects with Lung Cancer with a High Level of LDH

Clinical trials have been performed to demonstrate the efficacy of erlotinib in the treatment of lung cancer, specifically locally advanced or metastatic non-small cell lung cancer (NSCLC). For example, the efficacy and safety of single-agent erlotinib was assessed in a randomized, double blind, placebo-controlled trial in 731 patients with locally advanced or metastatic NSCLC after failure of at least one chemotherapy regimen. Patients were randomized 2:1 to receive erlotinib 150 mg or placebo (488 erlotinib, 243 placebo) orally once daily until disease progression or unacceptable toxicity. Study endpoints included overall survival, response rate, and progression-free survival (PFS). Duration of response was also examined. The primary endpoint was survival. Fifty percent of the patients had received only one prior regimen of chemotherapy. About three quarters of these patients were known to have smoked at some time. Erlotinib was demonstrated to increase survival vs. placebo (6.7. months vs. 4.7 months), to increase the rate of one year survival (31.2% vs. 21.2%), to increase progression free survival (9.9 weeks vs. 7.9 weeks); increase tumor response (8.9% vs. 0.9%), and increase response duration (median 34.3 weeks vs. 15.9 weeks). The results were found to be statistically significant.

In another randomized, double-blind, placebo-controlled trial of 889 subjects with locally advanced or metastatic NSCLC whose disease did not progress during first line platinum-based chemotherapy, the efficacy and safety of erlotinib as maintenance treatment of NSCLC were demonstrated. Subjects were randomized 1:1 to receive erlotinib 150 mg or placebo orally once daily (438 erlotinib, 451 placebo) until disease progression or unacceptable toxicity. The primary objective of the study was to determine if the administration of erlotinib after standard platinum-based chemotherapy in the treatment of NSCLC resulted in improved progression free survival (PFS) when compared with placebo, in all patients or in patients with EGFR immunohistochemistry (IHC) positive tumors. Progression free survival was significantly longer in the erlotinib group vs. the placebo group (2.8 months vs. 2.6 months). Although a difference in overall survival was also noted (12 weeks vs. 11 weeks), the difference was not statistically significant.

A chart review is performed to determine if levels of one or more hypoxic markers, particularly LDH, were analyzed for the subjects prior to, and optionally during treatment with a regimen including erlotinib. If no information is available regarding the levels of hypoxic markers, serum samples retained from the study subjects are analyzed for LDH level and outcomes are analyzed in view of the LDH level.

Preliminarily, subjects within each of the groups, or at least the groups in which subjects were treated with a regimen including erlotinib, are divided into high and low LDH level based on the upper limit of normal (ULN) for the site where the testing is done. A value equal to or less than the ULN is considered as low. Values greater than the ULN are considered high. Alternatively, low LDH can be considered as levels up to and including 0.8 ULN with high LDH being considered all values above 0.8 ULN. Alternatively, low LDH can be considered as levels up to and including 1.2 or 1.5 ULN with high LDH being considered all values above 1.2 or 1.5 ULN, respectively. It may be possible to further stratify the high and low ULN groups to provide further predictive power of the LDH level in predicting the response of a subject to treatment with erlotinib, e.g., assigning those with an LDH level of 1 to <2 times, or 1 to <3 times, etc. the ULN as having an intermediate or slightly elevated LDH level. Ratios of LDH isoforms or subunits, e.g., ratios of the ULN values of LDHA to LDHB or LDH4 and/or LDH5 to LDH1 can also be used to determine high and low levels of hypoxia. Other cut-off values such as those provided in the instant application can also be selected. Statistical analysis can be used to select appropriate cut-offs. The outcome of the analysis is further used to select treatment regimens for subjects including or not including erlotinib based on the ULN level. The outcome of the analysis is further used to allow for the selection of subjects likely to benefit from treatment with erlotinib based on the ULN level. Subjects with a high level of LDH are selected for treatment with erlotinib as they are likely to benefit from such treatment. Subjects with a low level of LDH are selected against for treatment with erlotinib as they are not likely to benefit from such treatment.

### Example 24

### Characterization of Treatment Outcomes to Demonstrate Improved Efficacy of Erlotinib in Subjects with Pancreatic Cancer with a High Level of LDH

Clinical trials have been performed to demonstrate the efficacy of erlotinib in the treatment of pancreatic cancer, specifically locally advanced, unresectable or metastatic pancreatic cancer. The efficacy and safety of erlotinib in combination with gemcitabine as a first-line treatment for pancreatic cancer was assessed in a randomized, double blind, placebo-controlled trial in 569 patients with locally advanced, unresectable or metastatic pancreatic cancer. Patients were randomized 1:1 to receive erlotinib (100 mg or 150 mg) or placebo once daily on a continuous schedule plus gemcitabine IV (1000 mg/m², Cycle 1—Days 1, 8, 15, 22, 29, 36 and 43 of an 8 week cycle; Cycle 2 and subsequent cycles—Days 1, 8 and 15 of a 4 week cycle at the approved dose and schedule for pancreatic cancer). Erlotinib or placebo was taken orally once daily until disease progression or unacceptable toxicity. The primary endpoint was survival. Secondary endpoints included response rate, and progression-free survival (PFS). Duration of response was also examined. A total of 285 patients were randomized to receive gemcitabine plus erlotinib (261 patients in the 100 mg cohort and 24 patients in the 150 mg cohort) and 284 patients were randomized to receive gemcitabine plus placebo (260 patients in the 100 mg cohort and 24 patients in the 150 mg cohort). Too few patients were treated in the 150 mg cohort to draw conclusions. Results for the 100 mg cohort demonstrated increased survival (6.4 months versus 6.0 months, p=0.028) and increased progression free survival (3.8 months versus 3.5 months, p=0.006) versus gemcitabine alone.

A chart review is performed to determine if levels of one or more hypoxic markers, particularly LDH, were analyzed for the subjects prior to, and optionally during treatment with a regimen including erlotinib. If no information is available regarding the levels of hypoxic markers, serum samples retained from the study subjects are analyzed for LDH level and outcomes are analyzed in view of the LDH level.

Preliminarily, subjects within each of the groups, or at least the groups in which subjects were treated with a regimen including erlotinib, are divided into high and low LDH level based on the upper limit of normal (ULN) for the site where the testing is done. A value equal to or less than the ULN is considered as low. Values greater than the ULN are considered high. Alternatively, low LDH can be considered as levels up to and including 0.8 ULN with high LDH being considered all values above 0.8 ULN. Alternatively, low LDH can be considered as levels up to and including 1.2 or 1.5 ULN with high LDH being considered all values above 1.2 or 1.5 ULN, respectively. It may be possible to further stratify the high and low ULN groups to provide further predictive power of the LDH level in predicting the response of a subject to treatment with erlotinib, e.g., assigning those with an LDH level of 1 to <2 times, or 1 to <3 times, etc. the ULN as having an intermediate or slightly elevated LDH level. Ratios of LDH isoforms or subunits, e.g., ratios of the ULN values of LDHA to LDHB or LDH4 and/or LDH5 to LDH1 or total LDH can also be used to determine high and low levels of hypoxia. Other cut-off values such as those provided in the instant application can also be selected. Statistical analysis can be used to select appropriate cut-offs. The outcome of the analysis is further used to select treatment regimens for subjects including or not including erlotinib based on the ULN level. The outcome of the analysis is further used to allow for the selection of subjects likely to benefit from treatment with erlotinib based on the ULN level. Subjects with a high level of LDH are selected for treatment with erlotinib as they are likely to benefit from such treatment. Subjects with a low level of LDH are selected against for treatment with erlotinib as they are not likely to benefit from such treatment.

### Example 25

### Trial to Demonstrate Improved Efficacy of Erlotinib in Subjects with Lung or Pancreatic Cancer with a High Level of LDH

Subjects are identified as having one of lung or pancreatic cancer. A subject is selected as being candidate for treatment with erlotinib based on appropriate inclusion or exclusion criteria. Routine assessments are made prior to treatment to characterize the disease state of the subject including, but not limited to, imaging studies, hematological studies, and physical examination. Additionally, coded serum sample from the subject is tested to determine the LDH level. The results from the LDH level determination are not matched to the subject until the end of the treatment period. However, samples can be tested to allow sufficient numbers of subjects with low and high LDH levels to be recruited to provide sufficient power to the study.

Subjects are treated with the standard dose of erlotinib, either alone or in combination with other agents. For example, the following protocols can be used for various cancer types:

### Lung Cancer:

Oral dose of 150 mg/day taken on an empty stomach.

### Pancreatic Cancer

Oral dose of 100 mg/day taken on an empty stomach.

At predetermined regular or irregular intervals, subjects are assessed for specific outcomes including, but not limited to, overall survival, progression free survival, time to progression, and adverse events. Treatment is continued for as long as the subject responds positively to treatment with erlotinib and there are no limiting adverse events.

Upon conclusion of the study, the results from the LDH level analysis are unblinded and matched to the subjects. As specific methods of testing are available, the amount of LDH is scored as being low or high based on the upper limit of normal (ULN) for the site where the testing is done. A value equal to or less than the ULN is considered as low. A value greater than the ULN is considered to be high. Alternatively, low LDH can be considered as levels up to and including 0.8 ULN with high LDH being considered all values above 0.8 ULN. Alternatively, low LDH can be considered as levels up to and including 1.2 or 1.5 ULN with high LDH being considered all values above 1.2 or 1.5 ULN, respectively. It may be possible to further stratify the high and low ULN groups to provide further predictive power of the LDH level in predicting the response of a subject to treatment with temsirolimus, e.g., assigning those with an LDH level of 1 to <2 times, or 1 to <3 times, etc. the ULN as having an intermediate or slightly elevated LDH level. Ratios of LDH isoforms or subunits, e.g., ratios of the ULN values of LDHA to LDHB or LDH4 and/or LDH5 to LDH1 or total LDH can also be used to determine high and low levels of hypoxia. Other cut-off values such as those provided in the instant application can also be selected. Statistical analysis can be used to select appropriate cut-offs. The outcome of the analysis is further used to select treatment regimens for subjects including or not including erlotinib based on the ULN level. The outcome of the analysis is further used to allow for the selection of subjects likely to benefit from treatment with erlotinib based on the ULN level. Subjects with a high level of LDH are selected for treatment with erlotinib as they are likely to benefit from such treatment. Subjects with a low level of LDH are selected against for treatment with erlotinib as they are not likely to benefit from such treatment.

### Example 26

### Selection of Subjects with Squamous Cell Carcinoma and a High Level of LDH for Treatment with Erlotinib

Subject is identified as having squamous cell carcinoma or other cancer type known to be or suspected to be susceptible to treatment with erlotinib, and being candidate for treatment with erlotinib. A serum sample from the subject is tested to determine the LDH level. The amount of LDH is scored as being low or high based on the upper limit of normal (ULN) for the site where the testing is done. A value equal to or less than the ULN is considered as low. A value greater than the ULN is considered to be high. Alternatively, low LDH can be considered as levels up to and including 0.8 ULN with high LDH being considered all values above 0.8 ULN. Alternatively, low LDH can be considered as levels up to and including 1.2 or 1.5 ULN with high LDH being considered all values above 1.2 or 1.5 ULN, respectively. It may be possible to further stratify the high and low ULN groups to provide further predictive power of the LDH level in predicting the response of a subject to treatment with temsirolimus, e.g., assigning those with an LDH level of 1 to <2 times, or 1 to <3 times, etc. the ULN as having an intermediate or slightly elevated LDH level. Ratios of LDH isoforms or subunits, e.g., ratios of the ULN values of LDHA to LDHB or LDH4 and/or LDH5 to LDH1 or total LDH can also be used to determine high and low levels of hypoxia. Other cut-off values such as those provided in the instant application can also be selected.

If the subject has a low LDH level, treatment with compounds other than erlotinib is selected. If the subject has a high LDH level, treatment with erlotinib, optionally with other agents, is selected as the treatment regimen.

### Example 27

### Characterization of Treatment Outcomes to Demonstrate Improved Efficacy of PTK787 in Subjects with Metastatic Colorectal Cancer with a High Level of LDH

Clinical studies have been performed to demonstrate the efficacy of PTK787 in the treatment of metastatic colorectal cancer (mCRC). For example, a randomized, double-blind, placebo-controlled phase III trial of PTK787 in 1168 patients with mCRC was performed (“CONFIRM 1”). Patients received PTK787 at a dosage of 1250 mg/day my mouth or placebo, with or without oxaliplatin, 5-FU or LV intravenously every two weeks. The study found that the effect of PTK787 was dependent on the serum level of LDH. See table, below.

A second randomized, double-blind, placebo-controlled phase III trial of PTK787 in 855 patients with mCRC pretreated with 5-FU/irinotecan was also performed (“CONFIRM 2”). Patients received PTK787 at a dosage of 1250 mg/day my mouth or placebo, with or without oxaliplatin, 5-FU or LV intravenously every two weeks. The study found that the effect of PTK787 was dependent on the serum level of LDH. See table, below.

PFS by High LDH, Low LDH and Overall

| | N | HR | P Value |
|---|---|---|---|
| CONFIRM 1 - High LDH | 316 | 0.61 | 0.002 |
| CONFIRM 2 - High LDH | 250 | 0.63 | <.0.001 |
| CONFIRM 1 - Low LDH | 852 | 0.93 | 0.43 |
| CONFIRM 2 - Low LDH | 605 | 0.95 | 0.58 |
| All CONFIRM 1 & 2 Patients | 2023 | 0.82 | <0.001 |
| All CONFIRM 1 & 2 High LDH | 566 | 0.62 | <0.001 |
| Patients | | | |

Accordingly, the treatment effect of PTK787 is observed in poor-prognosis patients with high serum LDH, indicating the role of LDH as a predictive biomarker for PTK787 therapy.

### Example 28

### Characterization of Treatment Outcomes to Demonstrate Improved Efficacy Of PTK787 in Subjects with Pancreatic Cancer with a High Level of LDH

Clinical studies have been performed to demonstrate the efficacy of PTK787 in the treatment of pancreatic cancer. For example, a phase I study of PTK787 and gemcitabine was performed. Gemcitabine was given by fixed-dose rate infusion weekly ×3 in a 28 day cycle, and vatalanib was given orally daily. The study found that six of eleven patients (55%) had stable disease as the best response, ranging from 2-6 months. See, e.g., Journal of Clinical Oncology, 2006 ASCO Annual Meeting Proceedings Part I. Vol 24, No. 18S (June 20 Supplement), 2006: 4122.

A chart review is performed to determine if levels of one or more hypoxic markers, particularly LDH, were analyzed for the subjects prior to, and optionally during treatment with a regimen including PTK787. If no information is available regarding the levels of hypoxic markers, serum samples retained from the study subjects are analyzed for LDH level and outcomes are analyzed in view of the LDH level.

Preliminarily, subjects within each of the groups, or at least the groups in which subjects were treated with a regimen including PTK787, are divided into high and low LDH level based on the upper limit of normal (ULN) for the site where the testing is done. A value equal to or less than the ULN is considered as low. Values greater than the ULN are considered high. Alternatively, low LDH can be considered as levels up to and including 0.8 ULN with high LDH being considered all values above 0.8 ULN. Alternatively, low LDH can be considered as levels up to and including 1.2 or 1.5 ULN with high LDH being considered all values above 1.2 or 1.5 ULN, respectively. It may be possible to further stratify the high and low ULN groups to provide further predictive power of the LDH level in predicting the response of a subject to treatment with PTK787, e.g., assigning those with an LDH level of 1 to <2 times, or 1 to <3 times, etc. the ULN as having an intermediate or slightly elevated LDH level. Ratios of LDH isoforms or subunits, e.g., ratios of the ULN values of LDHA to LDHB or LDH4 and/or LDH5 to LDH1 or total LDH can also be used to determine high and low levels of hypoxia. Other cut-off values such as those provided in the instant application can also be selected. Statistical analysis can be used to select appropriate cut-offs. The outcome of the analysis is further used to select treatment regimens for subjects including or not including PTK787 based on the ULN level. The outcome of the analysis is further used to allow for the selection of subjects likely to benefit from treatment with PTK787 based on the ULN level.

Subjects with a high level of LDH are selected for treatment with PTK787 as they are likely to benefit from such treatment. Subjects with a low level of LDH are selected against for treatment with PTK787 as they are not likely to benefit from such treatment.

### Example 29

### Trial to Demonstrate Improved Efficacy of PTK787 in Subjects with Head and Neck Cancer with a High Level of LDH

Subjects are identified as having head and neck cancer, or other cancer type known to be or suspected to be susceptible to treatment with PTK787. A subject is selected as being candidate for treatment with PTK787 based on appropriate inclusion or exclusion criteria. Routine assessments are made prior to treatment to characterize the disease state of the subject including, but not limited to, imaging studies, hematological studies, and physical examination. Additionally, coded serum sample from the subject is tested to determine the LDH level. The results from the LDH level determination are not matched to the subject until the end of the treatment period. However, samples can be tested to allow sufficient numbers of subjects with low and high LDH levels to be recruited to provide sufficient power to the study.

Subjects are treated with the standard dose of PTK787, either alone or in combination with other agents. Typically, PTK787 is dosed at 1250 mg/day orally. Initiation of further rounds of administration is based on subject response and adverse events.

At predetermined regular or irregular intervals, subjects are assessed for specific outcomes including, but not limited to, overall survival, progression free survival, time to progression, and adverse events. Treatment is continued for as long as the subject responds positively to treatment with PTK787 and there are no limiting adverse events.

Upon conclusion of the study, the results from the LDH level analysis are unblinded and matched to the subjects. As specific methods of testing are available, the amount of LDH is scored as being low or high based on the upper limit of normal (ULN) for the site where the testing is done. A value equal to or less than the ULN is considered as low. A value greater than the ULN is considered to be high. Alternatively, low LDH can be considered as levels up to and including 0.8 ULN with high LDH being considered all values above 0.8 ULN. Alternatively, low LDH can be considered as levels up to and including 1.2 or 1.5 ULN with high LDH being considered all values above 1.2 or 1.5 ULN, respectively. It may be possible to further stratify the high and low ULN groups to provide further predictive power of the LDH level in predicting the response of a subject to treatment with PTK787, e.g., assigning those with an LDH level of 1 to <2 times, or 1 to <3 times, etc. the ULN as having an intermediate or slightly elevated LDH level. Ratios of LDH isoforms or subunits, e.g., ratios of the ULN values of LDHA to LDHB or LDH4 and/or LDH5 to LDH1 or total LDH can also be used to determine high and low levels of hypoxia. Other cut-off values such as those provided in the instant application can also be selected. Statistical analysis can be used to select appropriate cut-offs. The outcome of the analysis is further used to select treatment regimens for subjects including or not including PTK787 based on the ULN level. The outcome of the analysis is further used to allow for the selection of subjects likely to benefit from treatment with PTK787 based on the ULN level. Subjects with a high level of LDH are selected for treatment with PTK787 as they are likely to benefit from such treatment. Subjects with a low level of LDH are selected against for treatment with PTK787 as they are not likely to benefit from such treatment.

### Example 30

### Characterization of Treatment Outcomes to Demonstrate Improved Efficacy of BEZ235 in Subjects with Breast Cancer with a High Level of LDH

Clinical studies have been performed to demonstrate the efficacy of BEZ235 in the treatment of breast cancer. For example, a phase I multi-center, open-label study of BEZ235 and trastuzumab, either alone or in combination is performed. BEZ235 will be administered orally on a continuous dosing schedule in adult patients with advanced solid malignancies, including patients with advanced breast cancer.

A chart review is performed to determine if levels of one or more hypoxic markers, particularly LDH, were analyzed for the subjects prior to, and optionally during treatment with a regimen including BEZ235. If no information is available regarding the levels of hypoxic markers, serum samples retained from the study subjects are analyzed for LDH level and outcomes are analyzed in view of the LDH level.

Preliminarily, subjects within each of the groups, or at least the groups in which subjects were treated with a regimen including BEZ235, are divided into high and low LDH level based on the upper limit of normal (ULN) for the site where the testing is done. A value equal to or less than the ULN is considered as low. Values greater than the ULN are considered high. Alternatively, low LDH can be considered as levels up to and including 0.8 ULN with high LDH being considered all values above 0.8 ULN. Alternatively, low LDH can be considered as levels up to and including 1.2 or 1.5 ULN with high LDH being considered all values above 1.2 or 1.5 ULN, respectively. It may be possible to further stratify the high and low ULN groups to provide further predictive power of the LDH level in predicting the response of a subject to treatment with BEZ235, e.g., assigning those with an LDH level of 1 to <2 times, or 1 to <3 times, etc. the ULN as having an intermediate or slightly elevated LDH level. Ratios of LDH isoforms or subunits, e.g., ratios of the ULN values of LDHA to LDHB or LDH4 and/or LDH5 to LDH1 or total LDH can also be used to determine high and low levels of hypoxia. Other cut-off values such as those provided in the instant application can also be selected. Statistical analysis can be used to select appropriate cut-offs. The outcome of the analysis is further used to select treatment regimens for subjects including or not including BEZ235 based on the ULN level. The outcome of the analysis is further used to allow for the selection of subjects likely to benefit from treatment with BEZ235 based on the ULN level.

Subjects with a high level of LDH are selected for treatment with BEZ235 as they are likely to benefit from such treatment. Subjects with a low level of LDH are selected against for treatment with BEZ235 as they are not likely to benefit from such treatment.

### Example 31

### Trial to Demonstrate Improved Efficacy of BEZ235 in Subjects with Solid Tumors with a High Level of LDH

Subjects are identified as having a solid tumor, or other cancer type known to be or suspected to be susceptible to treatment with BEZ235. A subject is selected as being candidate for treatment with BEZ235 based on appropriate inclusion or exclusion criteria. Routine assessments are made prior to treatment to characterize the disease state of the subject including, but not limited to, imaging studies, hematological studies, and physical examination. Additionally, coded serum sample from the subject is tested to determine the LDH level. The results from the LDH level determination are not matched to the subject until the end of the treatment period. However, samples can be tested to allow sufficient numbers of subjects with low and high LDH levels to be recruited to provide sufficient power to the study.

Subjects are treated with the standard dose of BEZ235, either alone or in combination with other agents. Typically, BEZ235 is dosed at 10 mg/day orally. Initiation of further rounds of administration is based on subject response and adverse events.

At predetermined regular or irregular intervals, subjects are assessed for specific outcomes including, but not limited to, overall survival, progression free survival, time to progression, and adverse events. Treatment is continued for as long as the subject responds positively to treatment with BEZ235 and there are no limiting adverse events.

Upon conclusion of the study, the results from the LDH level analysis are unblinded and matched to the subjects. As specific methods of testing are available, the amount of LDH is scored as being low or high based on the upper limit of normal (ULN) for the site where the testing is done. A value equal to or less than the ULN is considered as low. A value greater than the ULN is considered to be high. Alternatively, low LDH can be considered as levels up to and including 0.8 ULN with high LDH being considered all values above 0.8 ULN. Alternatively, low LDH can be considered as levels up to and including 1.2 or 1.5 ULN with high LDH being considered all values above 1.2 or 1.5 ULN, respectively. It may be possible to further stratify the high and low ULN groups to provide further predictive power of the LDH level in predicting the response of a subject to treatment with BEZ235, e.g., assigning those with an LDH level of 1 to <2 times, or 1 to <3 times, etc. the ULN as having an intermediate or slightly elevated LDH level. Ratios of LDH isoforms or subunits, e.g., ratios of the ULN values of LDHA to LDHB or LDH4 and/or LDH5 to LDH1 or total LDH can also be used to determine high and low levels of hypoxia. Other cut-off values such as those provided in the instant application can also be selected. Statistical analysis can be used to select appropriate cut-offs. The outcome of the analysis is further used to select treatment regimens for subjects including or not including BEZ235 based on the ULN level. The outcome of the analysis is further used to allow for the selection of subjects likely to benefit from treatment with BEZ235 based on the ULN level. Subjects with a high level of LDH are selected for treatment with BEZ235 as they are likely to benefit from such treatment. Subjects with a low level of LDH are selected against for treatment with BEZ235 as they are not likely to benefit from such treatment.

### Example 32

### Selection of Subjects with Solid Tumors or Breast Cancer and a High Level of LDH for Treatment with BEZ235

Subject is identified as having a solid tumor, breast cancer, or other cancer type known to be or suspected to be susceptible to treatment with BEZ235, and being candidate for treatment with BEZ235. A serum sample from the subject is tested to determine the LDH level. The amount of LDH is scored as being low or high based on the upper limit of normal (ULN) for the site where the testing is done. A value equal to or less than the ULN is considered as low. A value greater than the ULN is considered to be high. Alternatively, low LDH can be considered as levels up to and including 0.8 ULN with high LDH being considered all values above 0.8 ULN. Alternatively, low LDH can be considered as levels up to and including 1.2 or 1.5 ULN with high LDH being considered all values above 1.2 or 1.5 ULN, respectively. It may be possible to further stratify the high and low ULN groups to provide further predictive power of the LDH level in predicting the response of a subject to treatment with BEZ235, e.g., assigning those with an LDH level of 1 to <2 times, or 1 to <3 times, etc. the ULN as having an intermediate or slightly elevated LDH level. Ratios of LDH isoforms or subunits, e.g., ratios of the ULN values of LDHA to LDHB or LDH4 and/or LDH5 to LDH1 or total LDH can also be used to determine high and low levels of hypoxia. Other cut-off values such as those provided in the instant application can also be selected.

If the subject has a low LDH level, treatment with compounds other than BEZ235 is selected. If the subject has a high LDH level, treatment with BEZ235, optionally with other agents, is selected as the treatment regimen.

### Example 33

### Characterization of Treatment Outcomes to Demonstrate Improved Efficacy of XL765 in Subjects with Malignant Gliomas with a High Level of LDH

Clinical studies are being performed to demonstrate the efficacy of XL765 in the treatment of malignant gliomas. For example, a phase I dose-escalation study is being performed using XL765 in combination with Temozolomide in adults with anaplastic gliomas or glioblastoma on a stable Temozolomide maintenance dose.

A chart review is performed to determine if levels of one or more hypoxic markers, particularly LDH, were analyzed for the subjects prior to, and optionally during treatment with a regimen including XL765. If no information is available regarding the levels of hypoxic markers, serum samples retained from the study subjects are analyzed for LDH level and outcomes are analyzed in view of the LDH level.

Preliminarily, subjects within each of the groups, or at least the groups in which subjects were treated with a regimen including XL765, are divided into high and low LDH level based on the upper limit of normal (ULN) for the site where the testing is done. A value equal to or less than the ULN is considered as low. Values greater than the ULN are considered high. Alternatively, low LDH can be considered as levels up to and including 0.8 ULN with high LDH being considered all values above 0.8 ULN. Alternatively, low LDH can be considered as levels up to and including 1.2 or 1.5 ULN with high LDH being considered all values above 1.2 or 1.5 ULN, respectively. It may be possible to further stratify the high and low ULN groups to provide further predictive power of the LDH level in predicting the response of a subject to treatment with XL765, e.g., assigning those with an LDH level of 1 to <2 times, or 1 to <3 times, etc. the ULN as having an intermediate or slightly elevated LDH level. Ratios of LDH isoforms or subunits, e.g., ratios of the ULN values of LDHA to LDHB or LDH4 and/or LDH5 to LDH1 or total LDH can also be used to determine high and low levels of hypoxia. Other cut-off values such as those provided in the instant application can also be selected. Statistical analysis can be used to select appropriate cut-offs. The outcome of the analysis is further used to select treatment regimens for subjects including or not including XL765 based on the ULN level. The outcome of the analysis is further used to allow for the selection of subjects likely to benefit from treatment with XL765 based on the ULN level.

### Example 34

### Characterization of Treatment Outcomes to Demonstrate Improved Efficacy of XL765 in Subjects with Solid Tumors with a High Level of LDH

Clinical studies are being performed to demonstrate the efficacy of XL765 in the treatment of solid tumors. For example, a non-randomized, uncontrolled, open-label phase I dose-escalation study is being performed using XL765. XL765 is administered twice daily using gelatin capsules supplied in 5 mg, 10 mg and 50 mg strengths, or is administered once daily using gelatin capsules supplied in 5 mg, 10 mg and 50 mg strengths.

A chart review is performed to determine if levels of one or more hypoxic markers, particularly LDH, were analyzed for the subjects prior to, and optionally during treatment with a regimen including XL765. If no information is available regarding the levels of hypoxic markers, serum samples retained from the study subjects are analyzed for LDH level and outcomes are analyzed in view of the LDH level.

Preliminarily, subjects within each of the groups, or at least the groups in which subjects were treated with a regimen including XL765, are divided into high and low LDH level based on the upper limit of normal (ULN) for the site where the testing is done. A value equal to or less than the ULN is considered as low. Values greater than the ULN are considered high. Alternatively, low LDH can be considered as levels up to and including 0.8 ULN with high LDH being considered all values above 0.8 ULN. Alternatively, low LDH can be considered as levels up to and including 1.2 or 1.5 ULN with high LDH being considered all values above 1.2 or 1.5 ULN, respectively. It may be possible to further stratify the high and low ULN groups to provide further predictive power of the LDH level in predicting the response of a subject to treatment with XL765, e.g., assigning those with an LDH level of 1 to <2 times, or 1 to <3 times, etc. the ULN as having an intermediate or slightly elevated LDH level. Ratios of LDH isoforms or subunits, e.g., ratios of the ULN values of LDHA to LDHB or LDH4 and/or LDH5 to LDH1 or total LDH can also be used to determine high and low levels of hypoxia. Other cut-off values such as those provided in the instant application can also be selected. Statistical analysis can be used to select appropriate cut-offs. The outcome of the analysis is further used to select treatment regimens for subjects including or not including XL765 based on the ULN level. The outcome of the analysis is further used to allow for the selection of subjects likely to benefit from treatment with XL765 based on the ULN level.

Subjects with a high level of LDH are selected for treatment with XL765 as they are likely to benefit from such treatment. Subjects with a low level of LDH are selected against for treatment with XL765 as they are not likely to benefit from such treatment.

### Example 35

### Trial to Demonstrate Improved Efficacy of XL765 in Subjects with Non-Small cell lung cancer with a high level of LDH

Subjects are identified as having non-small cell lung cancer, or other cancer type known to be or suspected to be susceptible to treatment with XL765. A subject is selected as being candidate for treatment with XL765 based on appropriate inclusion or exclusion criteria. Routine assessments are made prior to treatment to characterize the disease state of the subject including, but not limited to, imaging studies, hematological studies, and physical examination. Additionally, coded serum sample from the subject is tested to determine the LDH level. The results from the LDH level determination are not matched to the subject until the end of the treatment period. However, samples can be tested to allow sufficient numbers of subjects with low and high LDH levels to be recruited to provide sufficient power to the study.

Subjects are treated with the standard dose of XL765, either alone or in combination with other agents. Typically, XL765 is dosed at between 5 mg and 30 mg, either once or twice per day, orally. Initiation of further rounds of administration is based on subject response and adverse events.

At predetermined regular or irregular intervals, subjects are assessed for specific outcomes including, but not limited to, overall survival, progression free survival, time to progression, and adverse events. Treatment is continued for as long as the subject responds positively to treatment with XL765 and there are no limiting adverse events.

Upon conclusion of the study, the results from the LDH level analysis are unblinded and matched to the subjects. As specific methods of testing are available, the amount of LDH is scored as being low or high based on the upper limit of normal (ULN) for the site where the testing is done. A value equal to or less than the ULN is considered as low. A value greater than the ULN is considered to be high. Alternatively, low LDH can be considered as levels up to and including 0.8 ULN with high LDH being considered all values above 0.8 ULN. Alternatively, low LDH can be considered as levels up to and including 1.2 or 1.5 ULN with high LDH being considered all values above 1.2 or 1.5 ULN, respectively. It may be possible to further stratify the high and low ULN groups to provide further predictive power of the LDH level in predicting the response of a subject to treatment with XL765, e.g., assigning those with an LDH level of 1 to <2 times, or 1 to <3 times, etc. the ULN as having an intermediate or slightly elevated LDH level. Ratios of LDH isoforms or subunits, e.g., ratios of the ULN values of LDHA to LDHB or LDH4 and/or LDH5 to LDH1 or total LDH can also be used to determine high and low levels of hypoxia. Other cut-off values such as those provided in the instant application can also be selected. Statistical analysis can be used to select appropriate cut-offs. The outcome of the analysis is further used to select treatment regimens for subjects including or not including XL765 based on the ULN level. The outcome of the analysis is further used to allow for the selection of subjects likely to benefit from treatment with XL765 based on the ULN level. Subjects with a high level of LDH are selected for treatment with XL765 as they are likely to benefit from such treatment. Subjects with a low level of LDH are selected against for treatment with XL765 as they are not likely to benefit from such treatment.

### Example 36

### Selection of Subjects with Solid Tumors or Breast Cancer and a High Level of LDH for Treatment with XL765

Subject is identified as having a solid tumor, breast cancer, or other cancer type known to be or suspected to be susceptible to treatment with XL765, and being candidate for treatment with XL765. A serum sample from the subject is tested to determine the LDH level. The amount of LDH is scored as being low or high based on the upper limit of normal (ULN) for the site where the testing is done. A value equal to or less than the ULN is considered as low. A value greater than the ULN is considered to be high. Alternatively, low LDH can be considered as levels up to and including 0.8 ULN with high LDH being considered all values above 0.8 ULN. Alternatively, low LDH can be considered as levels up to and including 1.2 or 1.5 ULN with high LDH being considered all values above 1.2 or 1.5 ULN, respectively. It may be possible to further stratify the high and low ULN groups to provide further predictive power of the LDH level in predicting the response of a subject to treatment with XL765, e.g., assigning those with an LDH level of 1 to <2 times, or 1 to <3 times, etc. the ULN as having an intermediate or slightly elevated LDH level. Ratios of LDH isoforms or subunits, e.g., ratios of the ULN values of LDHA to LDHB or LDH4 and/or LDH5 to LDH1 or total LDH can also be used to determine high and low levels of hypoxia. Other cut-off values such as those provided in the instant application can also be selected.

If the subject has a low LDH level, treatment with compounds other than XL765 is selected. If the subject has a high LDH level, treatment with XL765, optionally with other agents, is selected as the treatment regimen.

### Example 37

### Characterization of Treatment Outcomes to Demonstrate Improved Efficacy of Pazopanib in Subjects with Colorectal Cancer with a High Level of LDH

Clinical trials have been performed to demonstrate the efficacy of pazopanib in the treatment of renal cell carcinoma (RCC).

A chart review is performed to determine if levels of one or more hypoxic markers, particularly LDH, were analyzed for the subjects prior to, and optionally during treatment with pazopanib. If no information is available regarding the levels of hypoxic markers, serum samples retained from the study subjects are analyzed for LDH level and outcomes are analyzed in view of the LDH level.

Preliminarily, subjects within each of the groups, or at least the groups in which subjects were treated with pazopanib, are divided into high and low LDH level based on the upper limit of normal (ULN) for the site where the testing is done. A value equal to or less than the ULN is considered as low. Values greater than the ULN are considered high. Alternatively, low LDH can be considered as levels up to and including 0.8 ULN with high LDH being considered all values above 0.8 ULN. Alternatively, low LDH can be considered as levels up to and including 1.2 or 1.5 ULN with high LDH being considered all values above 1.2 or 1.5 ULN, respectively. It may be possible to further stratify the high and low ULN groups to provide further predictive power of the LDH level in predicting the response of a subject to treatment with bevacizumab, e.g., assigning those with an LDH level of 1 to <2 times, or 1 to <3 times, etc. the ULN as having an intermediate or slightly elevated LDH level. Ratios of LDH isoforms or subunits, e.g., ratios of the ULN values of LDHA to LDHB or LDH4 and/or LDH5 to LDH1 or total LDH can also be used to determine high and low levels of hypoxia. Other cut-off values such as those provided in the instant application can also be selected. Statistical analysis can be used to select appropriate cut-offs. The outcome of the analysis is further used to select treatment regimens for subjects including or not including pazopanib based on the ULN level. The outcome of the analysis is further used to allow for the selection of subjects likely to benefit from treatment with pazopanib based on the ULN level. Subjects with a high level of LDH are selected for treatment with pazopanib as they are likely to benefit from such treatment. Subjects with a low level of LDH are selected against for treatment with pazopanib as they are not likely to benefit from such treatment.

### Example 38

### Trial to Demonstrate Improved Efficacy of Pazopanib in Subjects with Solid Tumors with a High Level of LDH

Subjects are identified as having a solid tumor. A subject is selected as being candidate for treatment with pazopanib based on appropriate inclusion and exclusion criteria. Routine assessments are made prior to treatment to characterize the disease state of the subject including, but not limited to, imaging studies, hematological studies, and physical examination. Additionally, coded serum sample from the subject is tested to determine the LDH level. The results from the LDH level determination are not matched to the subject until the end of the treatment period. However, samples can be tested to allow sufficient numbers of subjects with low and high LDH levels to be recruited to provide sufficient power to the study.

Subjects are treated with a regimen including pazopanib. Depending on the number of subjects available and the scope of the trial, the two regimens can be compared, or all subjects can be administered a single regimen. At predetermined regular or irregular intervals, subjects are assessed for specific outcomes including, but not limited to, overall survival, progression free survival, time to progression, and adverse events. Treatment is continued for as long as the subject responds positively to treatment with the assigned regimen and there are no limiting adverse events. However, an arbitrary treatment window can be selected to allow for conclusion of the trial.

Upon conclusion of the study, the results from the LDH level analysis are unblinded and matched to the subjects. As specific methods of testing are available, the amount of LDH is scored as being low or high based on the upper limit of normal (ULN) for the site where the testing is done. A value equal to or less than the ULN is considered as low. Values greater than the ULN are considered high. Alternatively, low LDH can be considered as levels up to and including 0.8 ULN with high LDH being considered all values above 0.8 ULN. Alternatively, low LDH can be considered as levels up to and including 1.2 or 1.5 ULN with high LDH being considered all values above 1.2 or 1.5 ULN, respectively. It may be possible to further stratify the high and low ULN groups to provide further predictive power of the LDH level in predicting the response of a subject to treatment with pazopanib, e.g., assigning those with an LDH level of 1 to <2 times, or 1 to <3 times, etc. the ULN as having an intermediate or slightly elevated LDH level. Ratios of LDH isoforms or subunits, e.g., ratios of the ULN values of LDHA to LDHB or LDH4 and/or LDH5 to LDH1 or total LDH can also be used to determine high and low levels of hypoxia. Other cut-off values such as those provided in the instant application can also be selected. Statistical analysis can be used to select appropriate cut-offs. The outcome of the analysis is further used to select treatment regimens for subjects including or not including pazopanib based on the ULN level. The outcome of the analysis is further used to allow for the selection of subjects likely to benefit from treatment with pazopanib based on the ULN level. Subjects with a high level of LDH are selected for treatment with pazopanib as they are likely to benefit from such treatment. Subjects with a low level of LDH are selected against for treatment with pazopanib as they are not likely to benefit from such treatment.

### Example 39

### Characterization of Treatment Outcomes to Demonstrate Improved Efficacy of Cediranib in Subjects with Colorectal Cancer with a High Level of LDH

Clinical trials have been performed to demonstrate the efficacy of cediranib in the treatment of renal cell carcinoma (RCC).

A chart review is performed to determine if levels of one or more hypoxic markers, particularly LDH, were analyzed for the subjects prior to, and optionally during treatment with cediranib. If no information is available regarding the levels of hypoxic markers, serum samples retained from the study subjects are analyzed for LDH level and outcomes are analyzed in view of the LDH level.

Preliminarily, subjects within each of the groups, or at least the groups in which subjects were treated with cediranib, are divided into high and low LDH level based on the upper limit of normal (ULN) for the site where the testing is done. A value equal to or less than the ULN is considered as low. Values greater than the ULN are considered high. Alternatively, low LDH can be considered as levels up to and including 0.8 ULN with high LDH being considered all values above 0.8 ULN. Alternatively, low LDH can be considered as levels up to and including 1.2 or 1.5 ULN with high LDH being considered all values above 1.2 or 1.5 ULN, respectively. It may be possible to further stratify the high and low ULN groups to provide further predictive power of the LDH level in predicting the response of a subject to treatment with bevacizumab, e.g., assigning those with an LDH level of 1 to <2 times, or 1 to <3 times, etc. the ULN as having an intermediate or slightly elevated LDH level. Ratios of LDH isoforms or subunits, e.g., ratios of the ULN values of LDHA to LDHB or LDH4 and/or LDH5 to LDH1 or total LDH can also be used to determine high and low levels of hypoxia. Other cut-off values such as those provided in the instant application can also be selected. Statistical analysis can be used to select appropriate cut-offs. The outcome of the analysis is further used to select treatment regimens for subjects including or not including cediranib based on the ULN level. The outcome of the analysis is further used to allow for the selection of subjects likely to benefit from treatment with cediranib based on the ULN level. Subjects with a high level of LDH are selected for treatment with cediranib as they are likely to benefit from such treatment. Subjects with a low level of LDH are selected against for treatment with cediranib as they are not likely to benefit from such treatment.

### Example 40

### Trial to Demonstrate Improved Efficacy of Cediranib in Subjects with Solid Tumors with a High Level of LDH

Subjects are identified as having a solid tumor. A subject is selected as being candidate for treatment with cediranib based on appropriate inclusion and exclusion criteria. Routine assessments are made prior to treatment to characterize the disease state of the subject including, but not limited to, imaging studies, hematological studies, and physical examination. Additionally, coded serum sample from the subject is tested to determine the LDH level. The results from the LDH level determination are not matched to the subject until the end of the treatment period. However, samples can be tested to allow sufficient numbers of subjects with low and high LDH levels to be recruited to provide sufficient power to the study.

Subjects are treated with a regimen including cediranib. Depending on the number of subjects available and the scope of the trial, the two regimens can be compared, or all subjects can be administered a single regimen. At predetermined regular or irregular intervals, subjects are assessed for specific outcomes including, but not limited to, overall survival, progression free survival, time to progression, and adverse events. Treatment is continued for as long as the subject responds positively to treatment with the assigned regimen and there are no limiting adverse events. However, an arbitrary treatment window can be selected to allow for conclusion of the trial.

Upon conclusion of the study, the results from the LDH level analysis are unblinded and matched to the subjects. As specific methods of testing are available, the amount of LDH is scored as being low or high based on the upper limit of normal (ULN) for the site where the testing is done. A value equal to or less than the ULN is considered as low. Values greater than the ULN are considered high. Alternatively, low LDH can be considered as levels up to and including 0.8 ULN with high LDH being considered all values above 0.8 ULN. Alternatively, low LDH can be considered as levels up to and including 1.2 or 1.5 ULN with high LDH being considered all values above 1.2 or 1.5 ULN, respectively. It may be possible to further stratify the high and low ULN groups to provide further predictive power of the LDH level in predicting the response of a subject to treatment with ganetespib, e.g., assigning those with an LDH level of 1 to <2 times, or 1 to <3 times, etc. the ULN as having an intermediate or slightly elevated LDH level. Ratios of LDH isoforms or subunits, e.g., ratios of the ULN values of LDHA to LDHB or LDH4 and/or LDH5 to LDH1 or total LDH can also be used to determine high and low levels of hypoxia. Other cut-off values such as those provided in the instant application can also be selected. Statistical analysis can be used to select appropriate cut-offs. The outcome of the analysis is further used to select treatment regimens for subjects including or not including cediranib based on the ULN level. The outcome of the analysis is further used to allow for the selection of subjects likely to benefit from treatment with cediranib based on the ULN level. Subjects with a high level of LDH are selected for treatment with cediranib as they are likely to benefit from such treatment. Subjects with a low level of LDH are selected against for treatment with cediranib as they are not likely to benefit from such treatment.

### Example 41

### Characterization of Treatment Outcomes to Demonstrate Improved Efficacy of Axitinib in Subjects with Colorectal Cancer with a High Level of LDH

Clinical trials have been performed to demonstrate the efficacy of axitinib in the treatment of colorectal cancer (CRC).

A chart review is performed to determine if levels of one or more hypoxic markers, particularly LDH, were analyzed for the subjects prior to, and optionally during treatment with axitinib. If no information is available regarding the levels of hypoxic markers, serum samples retained from the study subjects are analyzed for LDH level and outcomes are analyzed in view of the LDH level.

Preliminarily, subjects within each of the groups, or at least the groups in which subjects were treated with axitinib, are divided into high and low LDH level based on the upper limit of normal (ULN) for the site where the testing is done. A value equal to or less than the ULN is considered as low. Values greater than the ULN are considered high. Alternatively, low LDH can be considered as levels up to and including 0.8 ULN with high LDH being considered all values above 0.8 ULN. Alternatively, low LDH can be considered as levels up to and including 1.2 or 1.5 ULN with high LDH being considered all values above 1.2 or 1.5 ULN, respectively. It may be possible to further stratify the high and low ULN groups to provide further predictive power of the LDH level in predicting the response of a subject to treatment with bevacizumab, e.g., assigning those with an LDH level of 1 to <2 times, or 1 to <3 times, etc. the ULN as having an intermediate or slightly elevated LDH level. Ratios of LDH isoforms or subunits, e.g., ratios of the ULN values of LDHA to LDHB or LDH4 and/or LDH5 to LDH1 or total LDH can also be used to determine high and low levels of hypoxia. Other cut-off values such as those provided in the instant application can also be selected. Statistical analysis can be used to select appropriate cut-offs. The outcome of the analysis is further used to select treatment regimens for subjects including or not including axitinib based on the ULN level. The outcome of the analysis is further used to allow for the selection of subjects likely to benefit from treatment with axitinib based on the ULN level. Subjects with a high level of LDH are selected for treatment with axitinib as they are likely to benefit from such treatment. Subjects with a low level of LDH are selected against for treatment with axitinib as they are not likely to benefit from such treatment.

### Example 42

### Trial to Demonstrate Improved Efficacy of Axitinib in Subjects with Various Cancers with a High Level of LDH

Subjects are identified as having hepatocellular carcinoma, solid tumors, lung cancer, malignant mesothelioma, renal cell cancer, adenocarcinoma, adrenocortical cancer, adrenal cortex neoplasms, nasopharyngeal carcinoma, soft tissue sarcoma, colorectal cancer, prostate cancer, melanoma, pancreatic cancer, gastric cancer, breast cancer, thyroid cancer, and acute myeloid leukemia (AML) or myelodysplastic syndrome. A subject is selected as being candidate for treatment with axitinib based on appropriate inclusion and exclusion criteria. Routine assessments are made prior to treatment to characterize the disease state of the subject including, but not limited to, imaging studies, hematological studies, and physical examination. Additionally, coded serum sample from the subject is tested to determine the LDH level. The results from the LDH level determination are not matched to the subject until the end of the treatment period. However, samples can be tested to allow sufficient numbers of subjects with low and high LDH levels to be recruited to provide sufficient power to the study.

Subjects are treated with a regimen including axitinib. Depending on the number of subjects available and the scope of the trial, the two regimens can be compared, or all subjects can be administered a single regimen. At predetermined regular or irregular intervals, subjects are assessed for specific outcomes including, but not limited to, overall survival, progression free survival, time to progression, and adverse events. Treatment is continued for as long as the subject responds positively to treatment with the assigned regimen and there are no limiting adverse events. However, an arbitrary treatment window can be selected to allow for conclusion of the trial.

Upon conclusion of the study, the results from the LDH level analysis are unblinded and matched to the subjects. As specific methods of testing are available, the amount of LDH is scored as being low or high based on the upper limit of normal (ULN) for the site where the testing is done. A value equal to or less than the ULN is considered as low. Values greater than the ULN are considered high. Alternatively, low LDH can be considered as levels up to and including 0.8 ULN with high LDH being considered all values above 0.8 ULN. Alternatively, low LDH can be considered as levels up to and including 1.2 or 1.5 ULN with high LDH being considered all values above 1.2 or 1.5 ULN, respectively. It may be possible to further stratify the high and low ULN groups to provide further predictive power of the LDH level in predicting the response of a subject to treatment with axitinib, e.g., assigning those with an LDH level of 1 to <2 times, or 1 to <3 times, etc. the ULN as having an intermediate or slightly elevated LDH level. Ratios of LDH isoforms or subunits, e.g., ratios of the ULN values of LDHA to LDHB or LDH4 and/or LDH5 to LDH1 or total LDH can also be used to determine high and low levels of hypoxia. Other cut-off values such as those provided in the instant application can also be selected. Statistical analysis can be used to select appropriate cut-offs. The outcome of the analysis is further used to select treatment regimens for subjects including or not including axitinib based on the ULN level. The outcome of the analysis is further used to allow for the selection of subjects likely to benefit from treatment with axitinib based on the ULN level. Subjects with a high level of LDH are selected for treatment with axitinib as they are likely to benefit from such treatment. Subjects with a low level of LDH are selected against for treatment with axitinib as they are not likely to benefit from such treatment.

### Example 43

### Method of Evaluating Activity Levels of LDH Isoforms in Subject Samples

Human tumor cell lines HCT116 (ATCC #CRL-247; Schroy P C, et al. Cancer 76: 201-209, 1995) and 786-O (ATCC #CRL-1932; Williams R D, et al. In Vitro 12: 623-627, 1976), were obtained from the American Type Culture Collection (Manassus, Va., USA) were cultured using routine methods until a sufficient number of cells were obtained for implantation. Studies were conducted on animals between 7 and 12 weeks of age at implantation. To implant HCT116 tumor cells into nude mice, the cells were trypsinized, washed in PBS and resuspended at a concentration of 75×10⁶ cells/ml in McCoy's modified medium with 50% of BD Matrigel® Basement Membrane Matrix (BD Biosciences®, Bedford, Mass., USA). To implant 786-O tumor cells into nude mice, the cells were trypsinized as above, washed in PBS and resuspended at a concentration of 75×10⁶ cells/ml in RPMI 1640 medium with 50% of BD Matrigel®Basement Membrane Matrix. Using a 27 gauge needle and 1 cc syringe, 0.1 ml of the cell suspension was injected into the corpus adiposum of nude mice. The corpus adiposum is a fat body located in the ventral abdominal vicera in the right quadrant of the abdomen at the juncture of the os coxae (pelvic bone) and the os femoris (femur). The location permits palpation and measurement of the tumors using external calipers. Tumor volumes (V) were calculated by caliper measurement of the width (W), length (L) and thickness (T) of tumors using the following formula: V=0.5236×(L×W×T). Animals were randomized into treatment groups so that the average tumor volumes of each group were similar at the start of dosing.

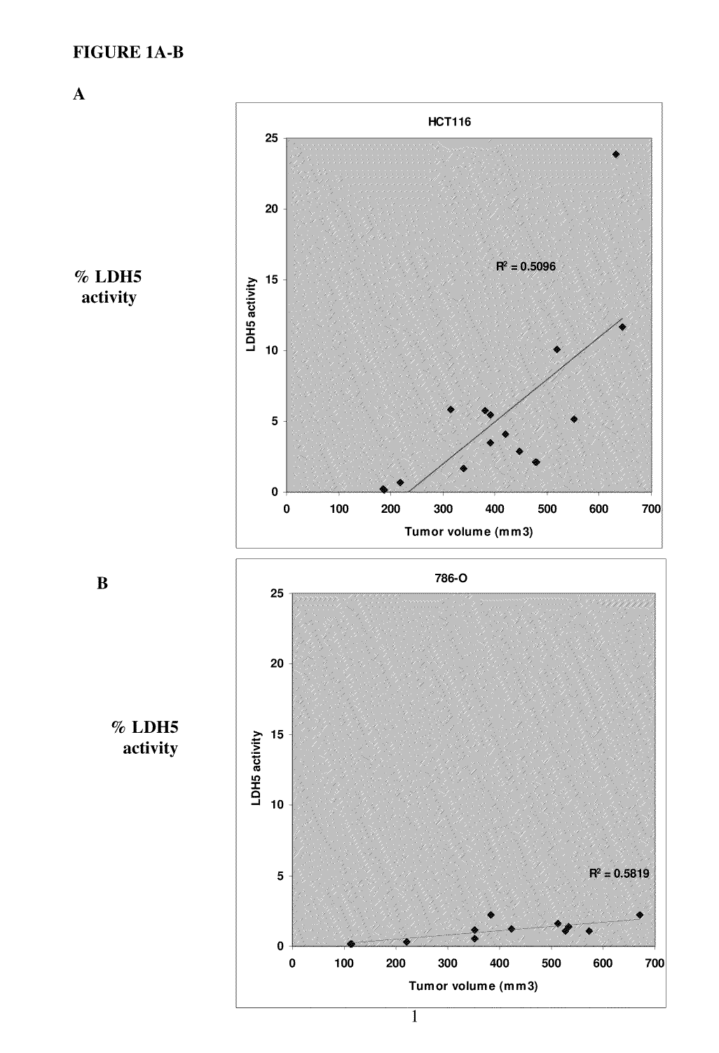

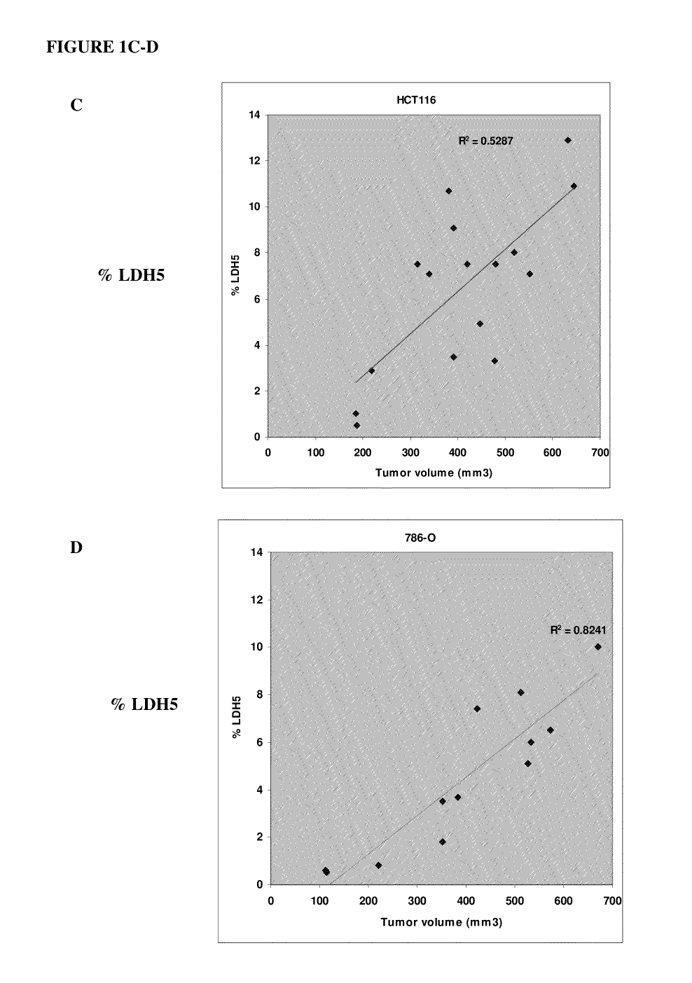

Blood was collected from the tumor bearing mice at appropriate time points, serum was prepared, and the serum frozen for later analysis. On the same days as blood collection, tumor volumes (V) were calculated by caliper measurement of the width (W), length (L) and thickness (T) of tumors using the following formula: V=0.5236×(L×W×T). After collection of the serum samples was completed, serum samples were resolved by gel electrophoresis. Following electrophoresis, the bands for the five isoenzymes were visualized by an enzymatic reaction using an in-gel assay. Lactate, nicotinamide adenine dinucleotide (NAD+), nitroblue tetrazolium (NBT), and phenazine methosulphate (PMS) were added to assess LDH activity. LDH converts lactate to pyruvate and reduces NAD+ to NADH. The hydrogens from NADH are transferred by PMS to NBT reducing it to a purple formazan dye. The percentage of each LDH isoenzyme activity as well as the relative amount of LDH5 was determined by densitometry (Beckman Appraise densitometer, Beckman Coulter Inc. or Sebia (GELSCAN, Sebia Inc). The percent of LDH5 protein and LDH5 activity relative to the total LDH present (i.e., the amount of LDH5, LDH5, LDH3, LDH2, and LDH1 combined) was calculated and graphed against tumor volume. The results are shown in FIGS. 1A-D.

FIGS. 1A and 1B show the amount of LDH5 activity as a percent of total LDH activity as determined by the in-gel assay. As shown, the HCT116 tumors had a substantially greater percent to LDH5 activity relative to total LDH activity as compared to the 786O tumors. FIGS. 1C and 1D demonstrate that despite the difference in the relative activity of LDH5 that is observed, the amount of LDH5 protein present relative to total LDH is about the same for both tumor types.

### Example 44

### Evaluation of Response of Hypoxic and Non-Hypoxic Tumors to Treatment with Various Chemotherapeutic Agents

A tumor model in which the tumors have relatively high and low levels of LDH5, indicating high and low levels of hypoxia, using human tumor cell lines HCT116 and 786-O was established as set forth in the previous example. Using the model, various chemotherapeutic agents were tested to determine if a difference in response would be observed in hypoxic vs. non-hypoxic tumors as demonstrated by relative LDH5 activity levels.

As above, HCT116 and 786-O cells were cultured and implanted into nude mice using the methods in the previous example. Tumor growth was measured using calipers. Prior to treatment with the various agents, tumors were permitted to develop in vivo until they reached approximately 150 mm³ in volume, which typically required 2-3 weeks following implantation. Animals were randomized into treatment groups so that the average tumor volumes of each group were similar at the start of dosing.

Mice were dosed with the agents as shown in the table below.

| | Proposed | | | |
|---|---|---|---|---|
| Agent | Mechanism | Dose | | Frequency |
| temsirolimus | mTOR inhibitor | 0.4 | mg/kg | 1 x/week, I.V. |
| XL765 | PI3K/mTOR | 30 | mg/kg | 5 x/week P.O. |
| | inhibitor | | | |
| erlotinib | EGFR inhibitor | 40 | mg/kg | 1x/week, P.O. |
| | | 25 | mg/kg | |
| | | 10 | mg/kg | |
| sorafenib | VEGFR inhibitor | 30 | mg/kg | 5 x/week P.O |
| | | 10 | mg/kg | |
| Sutent | VEGFR inhibitor | 25 | mg/kg | 5x/week P.O. |
| | | 10 | mg/kg | |
| BEZ235 | PI3K/mTOR | 10 | mg/kg | 5x/week, P.O. |
| | inhibitor | | | |
| vatalanib | VEGFR inhibitor | 50 | mg/kg | 5x/week, P.O |
| bevacizumab | Anti-VEGF | 4 | mg/kg | 3x/week, I.P. |
| | | 1 | mg/kg | |
| cetuximab | Anti-EGFR | 1 | mg/kg | 2x/week, I.P. |
| | | 0.25 | mg/kg | |
| | | 0.08 | mg/kg | |
| panitumumab | Anti-EGFR | 1 | mg/kg | 2x/week, I.P. |
| | | 0.2 | mg/kg | |
| | | 0.05 | mg/kg | |

Tumor volume was monitored throughout the course of the study, until up to about 40 days from the date of tumor implantation. The exact number of days of the study depended on a number of factors including, for example, the number of days from implantation for the tumors to reach the desired volume. The study demonstrated that Erlotinib, XL765, valatanib, and bevacizumab were more effective at slowing tumor growth in tumors with high levels of hypoxia, i.e., the HCT116 tumors, than tumors with low levels of hypoxia, i.e., the 786O tumors.

### Bevacizumab

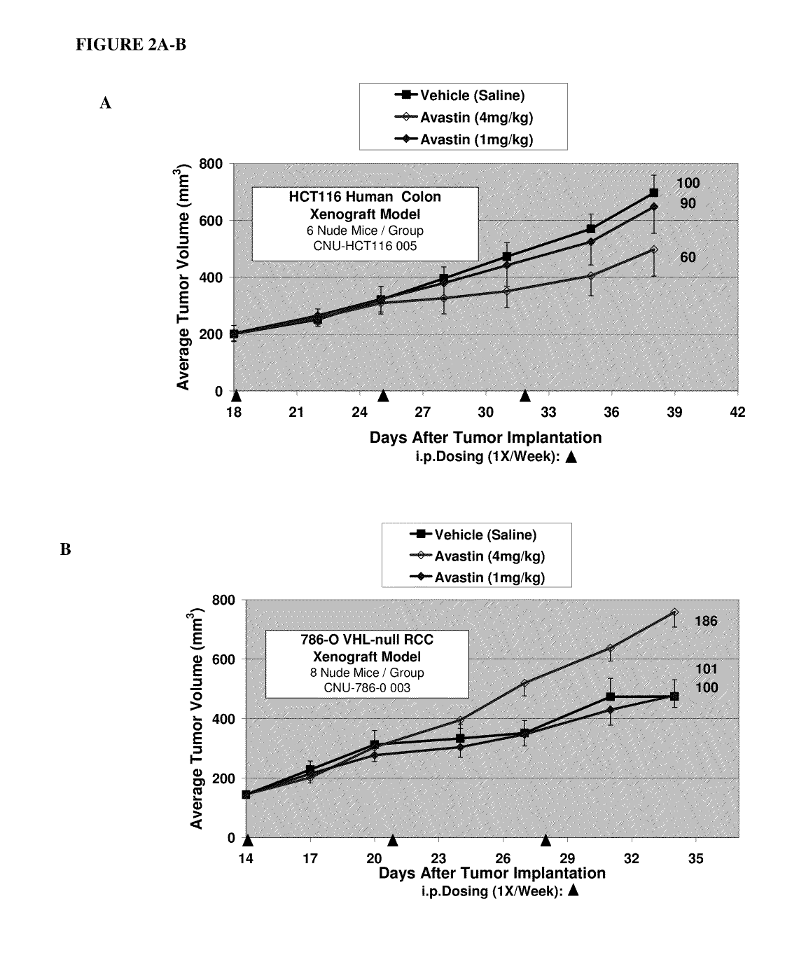

Exemplary results from animals treated with bevacizumab (Avastin®) are shown in FIGS. 2A-2B. The average tumor volume for each of the bevacizumab doses and untreated control was graphed against the number of days after tumor implantation. Growth curves have been plotted. Bevacizumab administration days are indicated by an upward pointing arrowhead. The % T/C (treatment/control) values for the last day of the experiment are shown at the end of each of the growth curves. FIG. 2A shows that in the HCT116 hypoxic tumor, the higher dose of bevacizumab (4 mg/kg) reduced the growth of the tumor as compared to control (p=0.0424) with a small trend to decreased tumor growth with treatment using the lower concentration of bevacizumab (p=0.1274). In the 786O tumors, the results are reversed. The greatest tumor burden is observed in the mice treated with the higher dose of bevacizumab (p=0.011) with no significant difference in tumor burden between low dose bevacizumab and control mice (p=0.437).

### Valatanib

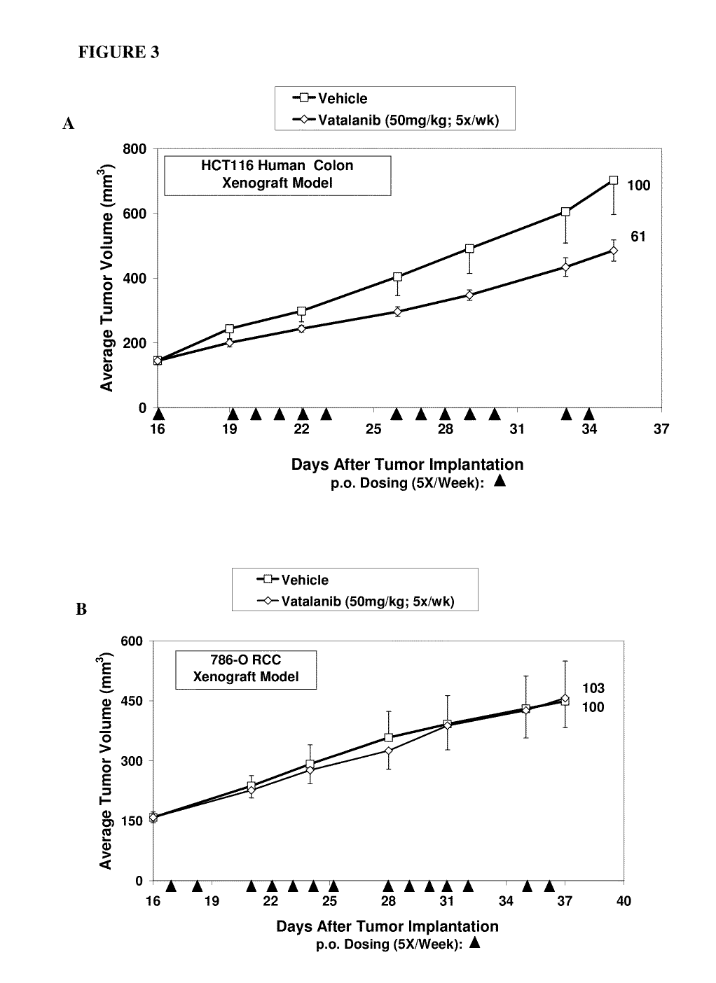

Exemplary results from animals treated with valatanib are shown in FIGS. 3A-3B. The average tumor volume for each of the valatanib treated and untreated control was graphed against the number of days after tumor implantation. Growth curves have been plotted. Valatinib administration days are indicated by an upward pointing arrowhead. The % T/C (treatment/control) values for the last day of the experiment are shown at the end of each of the growth curves. FIG. 3A shows that in the HCT116 hypoxic tumor, valatanib reduced the growth of the tumor as compared to control (p=0.1209). In the 786O tumors, there is no difference in tumor burden between the valatanib and control groups (p=0.7805).

### XL765

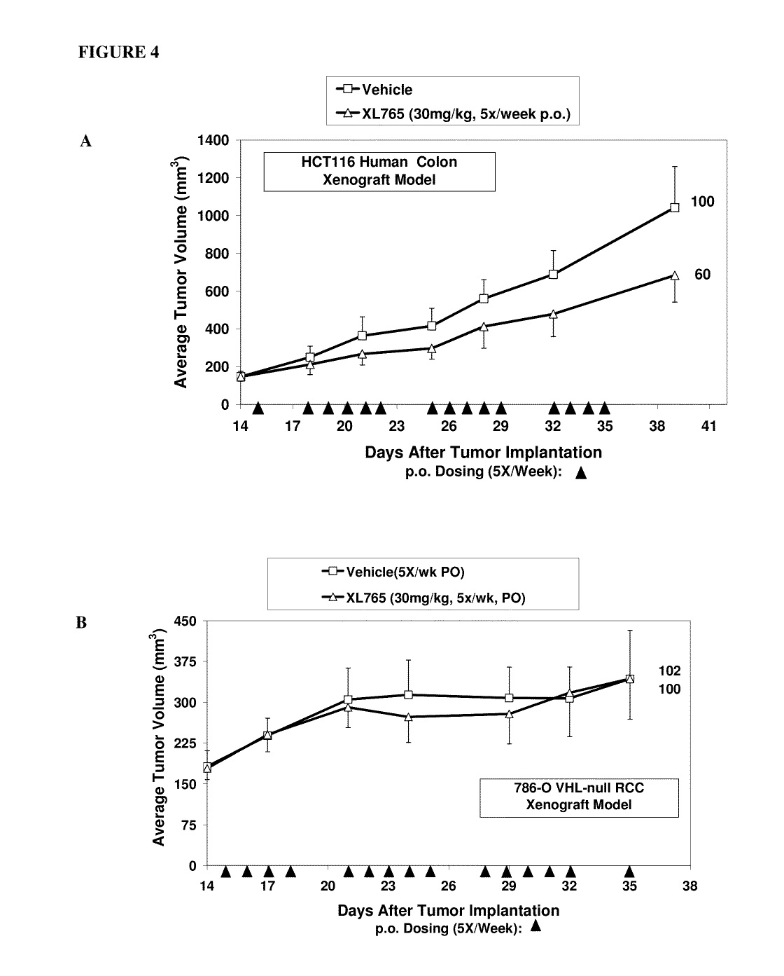

Exemplary results from animals treated with XL765 are shown in FIGS. 4A-4B. The average tumor volume for each of the XL765 treated and untreated control was graphed against the number of days after tumor implantation. Growth curves have been plotted. XL765 administration days are indicated by an upward pointing arrowhead. The % T/C (treatment/control) values for the last day of the experiment are shown at the end of each of the growth curves. FIG. 4A shows that in the HCT116 hypoxic tumor, XL765 reduced the growth rate of the tumor as compared to control (p=0.009). In the 786O tumors, there is no difference in tumor burden between the valatanib and control groups (p=0.7682).

### Erlotinib

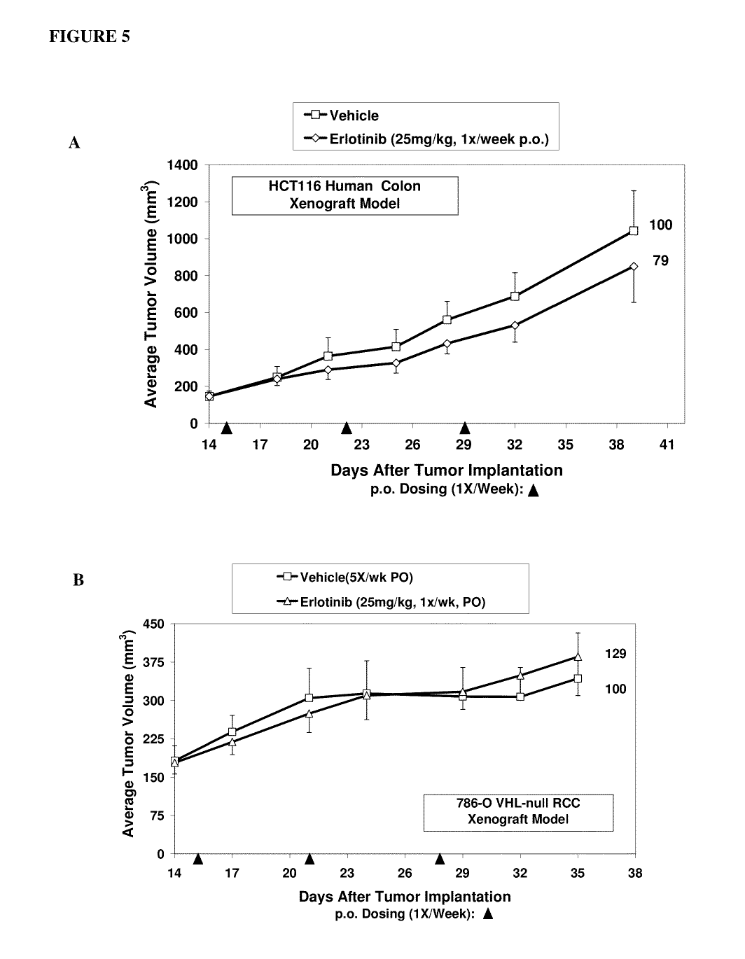

Exemplary results from animals treated with erlotinib are shown in FIGS. 5A-5B. The average tumor volume for each of the erlotinib treated and untreated control was graphed against the number of days after tumor implantation. Growth curves have been plotted. Erlotinib administration days are indicated by an upward pointing arrowhead. The % T/C (treatment/control) values for the last day of the experiment are shown at the end of each of the growth curves. FIG. 5A shows that in the HCT116 hypoxic tumor, erlotinib reduced the growth rate of the tumor as compared to control (p=0.0224). In the 786O tumors, there is no difference in tumor burden between the valatanib and control groups (p=0.8548).

Temsirolimus, sorafenib, sutent, BEZ235, cetuximab, panitumumab, and ganetespib were not found to work better in the tumors with a higher level of hypoxia, i.e., the HCT116 tumors.

### Incorporation by Reference

All publications, patents, and patent applications mentioned in this specification are herein incorporated by reference to the same extent as if each independent publication or patent application was specifically and individually indicated to be incorporated by reference.

### EQUIVALENTS

Those skilled in the art will recognize, or be able to ascertain using no more that routine experimentation, many equivalents to the specific embodiments of the invention described herein. Such equivalents are intended to be encompassed by the following claims.

1. A method of treating a subject having cancer comprising:
administering an agent selected from the group consisting of bevacizumab, STA-9090, temsirolimus, erlotinib, sorafenib, sunitinib, PTK787, BEZ235, XL765, pazopanib, cediranib, and axitinib to the subject, wherein the cancer comprises a tumor with a high level of hypoxia.

2. The method of claim 1, wherein the level of hypoxia in the tumor is determined in a subject sample.

3. The method of claim 1, wherein the level of hypoxia in the tumor is determined by detecting the activity level or expression level of one or more hypoxia modulated polypeptides.

4. The method of claim 3, wherein the activity level or expression level of the one or more hypoxia modulated polypeptides are up regulated in the sample.

5. The method of claim 1, wherein the level of hypoxia is determined by detecting the activity level or expression level of one or more hypoxia modulated polypeptides or using detection methods selected from the group consisting of detection of activity or expression of at least one isoform or subunit of lactate dehydrogenase (LDH), at least one isoform or subunit of hypoxia inducible factor (HIF), at least one pro-angiogenic form of vascular endothelial growth factor (VEGF), phosphorylated VEOF receptor (pKDR) 1, 2, and 3; neurolipin 1 (NRP-1), pyruvate dehydrokinase (PDH-K), ornithine decarboxylase (ODC), glucose transporter-1 (GLUT-1), glucose transporter-2 (GLUT-2), tumor size, blood flow, EF5 binding, pimonidazole binding, PET scan, and probe detection of hypoxia level.

6. The method of claim 5, wherein the isoform or subunit of LDH comprises one or more selected from the group consisting of LDH5, LDH14, LDH3, LDH2, LDH1, LDHA and LDHB; or any combination thereof including total LDH.

7. The method of claim 1, wherein detection of a high level of activity or expression of at least one LDH isoform or subunit comprises detection of an LDH activity or expression level of an LDH selected from the group consisting of total LDH, LDH5, LDH4, LDH5 plus LDH4, LDH5 plus LDH4 plus LDH3, and LDHA, wherein the activity level or expression level is 0.8 ULN or more.

8. The method of claim 1, wherein detection of a high level of activity or expression of at least one LDH isoform or subunit comprises detection of an LDH activity or expression level of an selected from the group consisting of total LDH, LDH5, LDH4, LDH5 plus LDH4, LDH5 plus LDH4 plus LDH3, and LDHA, wherein the activity level or expression level is 1.0 ULN or more.

9. The method of claim 1, wherein a high level of hypoxia comprises a ratio or a normalized ratio of 1.0 or more of the ULN, wherein the ratio or normalized ratio is selected from the group consisting of the LDHA to LDHB, LDH5 or LDH4 to LDH1, LDH5 or LDH4 to total LDH, LDH5 and LDH4 to LDH5 and LDH4 to total LDH, LDH5, LDH4, and LDH3 to LDH1, and LDH5, LDH4, and LDH3 to total LDH.

10. The method of claim 1, wherein the subject was previously treated with another chemotherapeutic agent.

11. The method of claim 1, further comprising identifying the subject as having a tumor with a high level of hypoxia.

12. A method for identifying a subject for treatment with an agent selected from the group consisting of bevacizumab, STA-9090, temsirolimus, erlotinib, sorafenib, sunitinib, PTK787, BEZ235, XL765, pazopanib, cediranib, and axitinib, comprising:
determining the level of hypoxia in a tumor from the subject, wherein a high level of hypoxia in the sample indicates the subject is likely to respond to therapy with an agent selected from the group consisting of bevacizumab, STA-9090, temsirolimus, erlotinib, sorafenib, sunitinib, PTK787, BEZ235, XL765, pazopanib, cediranib, and axitinib.

13. The method of claim 12, wherein a subject having a low level of hypoxia in the tumor is not likely to respond to therapy with an agent selected from the group consisting of bevacizumab, STA-9090, temsirolimus, erlotinib, sorafenib, sunitinib, PTK787, BEZ235, XL765, pazopanib, cediranib, and axitinib.

14-15. (canceled)

16. The method of claim 15, wherein the activity level or expression level of the one or more hypoxia modulated polypeptides are up regulated in the sample.

17. The method of claim 12, wherein the level of hypoxia is determined by detecting the activity level or expression level of one or more hypoxia modulated polypeptides or using detection methods selected from the group consisting of detection of activity or expression of at least one isoform or subunit of lactate dehydrogenase (LDH), at least one isoform or subunit of hypoxia inducible factor (HIF), at least one pro-angiogenic form of vascular endothelial growth factor (VEGF), phosphorylated VEGF receptor (pKDR) 1, 2, and 3; neurolipin 1 (NRP-1), pyruvate dehydrokinase (PDH-K), ornithine decarboxylase (ODC), glucose transporter-1(GLUT-1), glucose transporter-2 (GLUT-2), tumor size, blood flow, EF5 binding, pimonidazole binding, PET scan, and probe detection of hypoxia level.

18-23. (canceled)

24. A kit for selecting a therapeutic regimen including an agent selected from the group consisting of bevacizumab, STA-9090, temsirolimus, erlotinib, sorafenib, sunitinib, PTK787, BEZ235, XL765, pazopanib, cediranib, and axitinib for the treatment of cancer comprising:
at least one reagent for determining the level of hypoxia of in a subject sample; wherein the level of hypoxia is used to select the treatment regimen including an agent selected from the group consisting of bevacizumab, STA-9090, temsirolimus, erlotinib, sorafenib, sunitinib, PTK787, BEZ235, XL765, pazopanib, cediranib, and axitinib.

25. The kit of claim 24, wherein a high level of hypoxia is indicative that a therapeutic regimen with an agent selected from the group consisting of bevacizumab, STA-9090, temsirolimus, erlotinib, sorafenib, sunitinib, PTK787, BEZ235, XL765, pazopanib, cediranib, and axitinib should be selected.

26-27. (canceled)

28. The kit of claim 24, Wherein the level of hypoxia is determined by detecting an activity level or an expression level of one or more hypoxia modulated peptides.

29. The kit of claim 28, wherein the activity level or expression level of the one or more hypoxia modulated polypeptides are up regulated in the sample.

30. The kit of claim 24, wherein the level of hypoxia is determined by detecting the activity level or expression level of one or more hypoxia modulated polypeptides or using detection methods selected from the group consisting of detection of activity or expression of at least one isoform or subunit of lactate dehydrogenase (LDH), at least one isoform or subunit of hypoxia inducible factor (HIF), at least one pro-angiogenic form of vascular endothelial growth factor (VEGF), phosphorylated VEGF receptor (pKDR) 1, 2, and 3; neurolipin 1 (NRP-1), pyruvate dehydrokinase (PDH-K), ornithine decarboxylase (ODC), glucose transporter-1 (GLUT-1), glucose transporter-2 (GLUT-2), tumor size, blood flow, EF5 binding, pimonidazole binding, PET scan, and probe detection of hypoxia level.

30-46. (canceled)
